(19) United States
(12) Patent Application Publication
(10) Pub. No.: US 2015/0376699 A1
(43) Pub. Date: Dec. 31, 2015

(54) METHODS OF DETECTING SEPSIS

(71) Applicant: CEPHEID, Sunnyvale, CA (US)

(72) Inventors: David Vilanova, Caraman (FR); Olivier Delfour, Caraman (FR)

(73) Assignee: CEPHEID, Sunnyvale, CA (US)

(21) Appl. No.: 14/727,596

(22) Filed: Jun. 1, 2015

Related U.S. Application Data
(62) Division of application No. 13/617,789, filed on Sep. 14, 2012, now abandoned.
(60) Provisional application No. 61/535,596, filed on Sep. 16, 2011.
(60) Provisional application No. 61/539,805, filed on Sep. 27, 2011.
(60) Provisional application No. 61/550,783, filed on Oct. 24, 2011.

(51) Int. Cl. C12Q 1/68

(52) U.S. Cl. CPC C12Q 1/6883; C12Q 1/6832; C12Q 2600/112

(57) ABSTRACT

Methods of sepsis in a sample from a patient are provided. Methods of detecting changes in expression of one or more RNAs associated with sepsis are also provided. Compositions and kits are also provided.

This application is a divisional of U.S. patent application Ser. No. 13/617,789, filed Sep. 14, 2012, which claims priority to U.S. Provisional Application Nos. 61/535,596, filed Sep. 16, 2011; 61/539,805, filed Sep. 27, 2011; and 61/550,783, filed Oct. 24, 2011, all of which are incorporated by reference herein in their entireties for any purpose.

### 1. BACKGROUND

Sepsis is the presence in the blood or other tissues of pathogenic microorganisms or their toxins combined with the host's inflammatory response, known as systemic inflammatory response syndrome (“SIRS”) caused by the infection. The immune response is mediated by a class of proteins called toll-like receptors (“TLR”) that recognize structurally-conserved molecules broadly shared by microorganisms but which are distinguishable from host molecules.

Once microorganisms have breached barriers such as the skin or intestinal tract, the body's TLRs recognize them and stimulate an immune response. Thus, in addition to symptoms caused by the microbial infection itself, sepsis is also characterized by symptoms of acute inflammation brought on by the host's immune response. These latter symptoms may include fever and elevated white blood cell count, or low white blood cell count and low body temperature. SIRS is characterized by hemodynamic compromise and resultant metabolic dysregulation, and may be accompanied by symptoms such as high heart rate, high respiratory rate and elevated body temperature. The immunological response also causes widespread activation of acute phase proteins, affecting the complement system and the coagulation pathways, which then cause damage to the vasculature and organs. Various neuroendocrine counter-regulatory systems are then activated as well, often compounding the problem.

Sepsis is often treated in the intensive care unit with intravenous fluids and antibiotics and/or antiviral compounds. Sepsis progresses quickly, however, and even with immediate and aggressive treatment, severe sepsis can lead to organ failure and death. With a mortality rate of around 29%, severe sepsis is estimated to cause 215,000 deaths per year in the United States, more than acute myocardial infarction, stroke or pneumonia. Early treatment of sepsis appears to be critical for survival, and the high mortality rate may be due to late diagnosis or misdiagnosis of sepsis. Detection of an underlying infection in a suspected case of sepsis can take 24 to 48 hours, however. As a consequence, antibiotics are often administered before infection has been confirmed, potentially leading to an increase in antibiotic resistance in hospitals.

Thus, there is a need for early molecular markers for sepsis.

### 2. SUMMARY

In some embodiments, methods of detecting the presence of sepsis in a subject are provided. In some embodiments, methods of facilitating the diagnosis of sepsis in a subject are provided. In some embodiments, a method comprises detecting of at least one, at least two, at least three, at least four, at least five, or at least six RNAs selected from 2548, IL18RAP, 14689, 14621, miR-342, 13629, 13719, and miR-150 in a sample from the subject. In some embodiments, a method comprises detecting the level of at least one RNA selected from 2548, IL18RAP, 14689, 14621, and miR-342 in a sample from the subject. In some such embodiments, detection of a level of 2548, 14689, miR-342, and/or miR-150 that is less than a normal level of the respective RNA indicates the presence of sepsis. In some embodiments, detection of a level of IL18RAP, 14621, 13629, and/or 13719 that is greater than a normal level of the respective RNA indicates the presence of sepsis in the subject. In some embodiments, a method comprises comparing the level of a RNA selected from 2548, IL18RAP, 14689, 14621, miR-342, 13629, 13719, and miR-150 in the sample to a normal level of the RNA. In some such embodiments, detection of a level of 2548, 14689, miR-342, and/or miR-150 that is less than a normal level of the respective RNA indicates the presence of sepsis. In some such embodiments, detection of a level of IL18RAP, 14621, 13629, and/or 13719 that is greater than a normal level of the respective RNA indicates the presence of sepsis in the subject. In some embodiments of methods of facilitating the diagnosis of sepsis in a subject, the method further comprises communicating the results of the detection to a medical practitioner for the purpose of determining whether the subject has sepsis.

In some embodiments, a method of detecting sepsis and/or a method of facilitating diagnosis of sepsis comprises detecting the level of at least one RNA selected from 13629, 13719, and miR-150. In some embodiments, detection of a level of 13629 or 13719 that is greater than a normal level of the respective RNA indicates the presence of sepsis in the subject. In some such embodiments, detection of a level of miR-150 that is less than a normal level of miR-150 indicates the presence of sepsis in the subject.

In some embodiments, a method comprises detecting the level of at least three RNAs selected from 13629, IL18RAP, 13719, miR-150, 2548, 14689, 14621, and miR-342. In some embodiments, a method comprises detecting the level of at least four RNAs selected from 13629, IL18RAP, 13719, miR-150, 2548, 14689, 14621, and miR-342. In some embodiments, a method comprises detecting the level of at least five RNAs selected from 13629, IL18RAP, 13719, miR-150, 2548, 14689, 14621, and miR-342. In some embodiments, a method comprises detecting the level of at least six RNAs selected from 13629, IL18RAP, 13719, miR-150, 2548, 14689, 14621, and miR-342.

In some embodiments, a method comprises detecting the levels of 13629 and miR-150. In some embodiments, a method comprises detecting the levels of 13629-L, 13629-R, and miR-150. In some embodiments, a method comprises detecting the levels of 13629, 2548, and 14689. In some embodiments, a method comprises detecting the levels of miR-150, 14689, and 13629. In some embodiments, a method comprises detecting the levels of 14689, miR-342, 13629, and miR-150. In some embodiments, a method comprises detecting the levels of IL18RAP and 13629. In some embodiments, a method comprises detecting the levels of IL18RAP and miR-150. In some embodiments, a method comprises detecting the levels of IL18RAP, 13629, and miR-150. In some embodiments, a method comprises detecting the levels of 13629 and miR-150. In some embodiments, a method comprises detecting the levels of 13629 and 14621. In some embodiments, a method comprises detecting the levels of 13629, 14621, and miR-150. In some embodiments, a method comprises detecting the levels of 13629, 14621, and IL18RAP. In some embodiments, a method comprises detecting the levels of 13629, 14621, miR-150, and IL18RAP.

In some embodiments, methods of detecting the presence of sepsis in a subject are provided, wherein the method comprises obtaining a sample from the subject, and providing the sample to a laboratory for detection of the level of at least one, at least two, at least three, at least four, at least five, or at least six RNAs selected from 2548, IL18RAP, 14689, 14621, miR-342, 13629, 13719, and miR-150 in the sample. In some embodiments, a method further comprises receiving from the laboratory a communication indicating the level of the at least one RNA. In some such embodiments, detection of a level of 2548, 14689, miR-342, and/or miR-150 that is less than a normal level of the respective RNA indicates the presence of sepsis. In some such embodiments, detection of a level of IL18RAP, 14621, 13629, and/or 13719 that is greater than a normal level of the respective RNA indicates the presence of sepsis in the subject. In some embodiments, the method further comprises providing the sample to a laboratory for detection of the level of at least one RNA selected from 13629, 13719, and miR-150. In some such embodiments, detection of a level of 13629 or 13719 that is greater than a normal level of the respective RNA indicates the presence of sepsis in the subject. In some embodiments, detection of a level of miR-150 that is less than a normal levels of miR-150 indicates the presence of sepsis in the subject.

In some embodiments, a method comprises providing the sample to a laboratory for detection of the level of at least three RNAs selected from 13629, IL18RAP, 13719, miR-150, 2548, 14689, 14621, and miR-342. In some embodiments, a method comprises providing the sample to a laboratory for detection of the level of at least four RNAs selected from 13629, IL18RAP, 13719, miR-150, 2548, 14689, 14621, and miR-342. In some embodiments, a method comprises providing the sample to a laboratory for detection of the level of at least five RNAs selected from 13629, IL18RAP, 13719, miR-150, 2548, 14689, 14621, and miR-342. In some embodiments, a method comprises providing the sample to a laboratory for detection of the level of at least six RNAs selected from 13629, IL18RAP, 13719, miR-150, 2548, 14689, 14621, and miR-342.

In some embodiments, a method comprises providing the sample to a laboratory for detection of the levels of 13629 and miR-150. In some embodiments, a method comprises providing the sample to a laboratory for detection of the levels of 13629-L, 13629-R, and miR-150. In some embodiments, a method comprises providing the sample to a laboratory for detection of the levels of 13629, 2548, and 14689. In some embodiments, a method comprises providing the sample to a laboratory for detection of the levels of miR-150, 14689, and 13629. In some embodiments, a method comprises providing the sample to a laboratory for detection of the levels of 14689, miR-342, 13629, and miR-150. In some embodiments, a method comprises providing the sample to a laboratory for detection of the levels of IL18RAP and 13629. In some embodiments, a method comprises providing the sample to a laboratory for detection of the levels of IL18RAP and miR-150. In some embodiments, a method comprises providing the sample to a laboratory for detection of the levels of IL18RAP, 13629, and miR-150. In some embodiments, a method comprises providing the sample to a laboratory for detection of the levels of 13629 and miR-150. In some embodiments, a method comprises providing the sample to a laboratory for detection of the levels of 13629 and 14621. In some embodiments, a method comprises providing the sample to a laboratory for detection of the levels of 13629, 14621, and miR-150. In some embodiments, a method comprises providing the sample to a laboratory for detection of the levels of 13629, 14621, and IL18RAP. In some embodiments, a method comprises providing the sample to a laboratory for detection of the levels of 13629, 14621, miR-150, and IL18RAP.

In some embodiments, 2548 is 2548-L. In some embodiments, 14689 is 14689-L. In some embodiments, miR-342 is miR-342-3p. In some embodiments, 13629 is selected from 13629-L and 13629-R. In some embodiments, 13629 is 13629-L. In some embodiments, 13629 is 13629-R. In some embodiments, 13719 is 13719-L. In some embodiments, 14621 is 14621-L.

In some embodiments, the detecting comprises RT-PCR. In some embodiments, the detecting comprises quantitative RT-PCR.

In some embodiments, the sample is a bodily fluid. In some embodiments, the bodily fluid is selected from blood, urine, sputum, saliva, mucus, and semen. In some embodiments, the sample is a blood sample.

In some embodiments, the detecting is carried out in one assay reaction. In some embodiments, the detecting is carried out in more than one assay reaction. In some embodiments, at least one first RNA is detected in a first assay reaction and at least one second RNA is detected in a second assay reaction. In some embodiments, at least one first RNA is a microRNA and at least one second RNA is an mRNA.

In any of the embodiments described herein, a method may further comprise treating a subject for sepsis. In some embodiments, treating comprises a treatment selected from administering one or more antibiotics, administering a vasopressor, administering fluids, and administering oxygen. In some embodiments, treating comprises administering one or more antibiotics. In some embodiments, at least one antibiotic is a broad-spectrum antibiotic. In some embodiments, the broad spectrum antibiotic is selected from amoxicillin, imipenem, levofloxacin, gatifloxacin, moxifloxacin, and ampicillin. In some embodiments, the time from sample collection to treatment is less than 5 hours or less than three hours.

In some embodiments, a subject has a cardiac condition. In some embodiments, the cardiac condition is selected from myocardial infarction, congestive heart failure, ischaemic heart disease, stable angina, unstable angina, acute coronary syndrome, pulmonary embolism, infective endocarditis, atrial fibrillation, recent angioplasty, recent coronary artery stent placement, and recent coronary artery bypass graft surgery. In some embodiments, when a subject has a cardiac condition, the method comprises detecting the level of 13629. In some embodiments, the method comprises detecting the levels of 13629-L and 13629-R. In some embodiments, the method comprises detecting the levels of one or more additional RNAs, such as at least one, at least two, at least three, at least four, at least five, or at least six RNAs selected from 2548, IL18RAP, 14689, 14621, miR-342, 13719, and miR-150. In some embodiments, the method comprises detecting the level of miR-150, with or without detection of one or more additional RNAs.

In some embodiments, use of at least one, at least two, at least three, at least four, at least five, or at least six RNAs selected from 2548, IL18RAP, 14689, 14621, miR-342, 13629, 13719, and miR-150, for detecting the presence of sepsis in a subject is provided. In some embodiments, use of 13629 and miR-150 for detecting the presence of sepsis in a subject are provided. In some embodiments, use of 13629-L, 13629-R, and miR-150 for detecting the presence of sepsis in a subject are provided. In some embodiments, use of 13629, 2548, and 14689 for detecting the presence of sepsis in a subject is provided. In some embodiments, use of miR-150, 14689, and 13629 for detecting the presence of sepsis in a subject is provided. In some embodiments, use of 14689, miR-342, 13629, and miR-150 for detecting the presence of sepsis in a subject is provided. In some embodiments, use of IL18RAP and 13629 for detecting the presence of sepsis in a subject is provided. In some embodiments, use of IL18RAP and miR-150 for detecting the presence of sepsis in a subject is provided. In some embodiments, use of IL18RAP, 13629, and miR-150 for detecting the presence of sepsis in a subject is provided. In some embodiments, use of 13629 and miR-150 for detecting the presence of sepsis in a subject is provided. In some embodiments, use of 13629 and 14621 for detecting the presence of sepsis in a subject is provided. In some embodiments, use of 13629, 14621, and miR-150 for detecting the presence of sepsis in a subject is provided. In some embodiments, use of 13629, 14621, and IL18RAP for detecting the presence of sepsis in a subject is provided. In some embodiments, use of 13629, 14621, miR-150, and IL18RAP for detecting the presence of sepsis in a subject is provided.

In some embodiments, compositions are provided. In some embodiments, a composition comprises a set of polynucleotides for detecting at least one, at least two, at least three, at least four, at least five, or at least six RNAs selected from 13629, IL18RAP, 13719, miR-150, 2548, 14689, 14621, and miR-342 are provided. In some embodiments, each polynucleotide comprises a sequence that is identical to or complementary to at least eight contiguous nucleotides of an RNA selected from 13629, IL18RAP, 13719, miR-150, 2548, 14689, 14621, and miR-342. In some embodiments, each polynucleotide comprises a sequence that is identical to or complementary to at least eight contiguous nucleotides of a different RNA selected from 13629, IL18RAP, 13719, miR-150, 2548, 14689, 14621, and miR-342. In some embodiments, the set of polynucleotides comprises a first polynucleotide for detecting 13629-L, a second polynucleotide for detecting 13629-R, and a third polynucleotide for detecting miR-150. In some embodiments, the set of polynucleotides is for detecting: a) 13629, 2548, and 14689; b) miR-150, 14689, and 13629; c) 14689, miR-342, 13629, and miR-150; d) IL18RAP and 13629; e) IL18RAP and miR-150; 0 IL18RAP, 13629, and miR-150; g) 13629 and 14621; h) 13629, 14621, and miR-150; i) 13629, 14621, and IL18RAP; or k) 13629, 14621, miR-150, and IL18RAP. In some embodiments, each polynucleotide comprises 8 to 100, 8 to 75, 8 to 50, 8 to 40, or 8 to 30 nucleotides.

In some embodiments, a composition further comprises RNAs of a sample from a subject. In some embodiments, a composition comprises cDNA reverse transcribed from RNAs of a sample from a subject. In some embodiments, the sample is selected from blood, urine, sputum, saliva, and mucus. In some embodiments, the sample is a blood sample. In some embodiments, the subject from whom the same was obtained is suspected of having sepsis.

In some embodiments, kits are provided. In some embodiments, a kit comprises a set of polynucleotides for detecting at least one, at least two, at least three, at least four, at least five, or at least six RNAs selected from 13629, IL18RAP, 13719, miR-150, 2548, 14689, 14621, and miR-342. In some embodiments, each polynucleotide comprises a sequence that is identical to or complementary to at least eight contiguous nucleotides of an RNA selected from 13629, IL18RAP, 13719, miR-150, 2548, 14689, 14621, and miR-342. In some embodiments, each polynucleotide comprises a sequence that is identical to or complementary to at least eight contiguous nucleotides of a different RNA selected from 13629, IL18RAP, 13719, miR-150, 2548, 14689, 14621, and miR-342. In some embodiments, the set of polynucleotides comprises a first polynucleotide for detecting 13629-L, a second polynucleotide for detecting 13629-R, and a third polynucleotide for detecting miR-150. In some embodiments, the set of polynucleotides is for detecting: a) 13629, 2548, and 14689; b) miR-150, 14689, and 13629; c) 14689, miR-342, 13629, and miR-150; d) IL18RAP and 13629; e) IL18RAP and miR-150; 0 IL18RAP, 13629, and miR-150; g) 13629 and 14621; h) 13629, 14621, and miR-150; i) 13629, 14621, and IL18RAP; or k) 13629, 14621, miR-150, and IL18RAP. In some embodiments, each polynucleotide comprises 8 to 100, 8 to 75, 8 to 50, 8 to 40, or 8 to 30 nucleotides.

Further embodiments and details of the inventions are described below

### 3. BRIEF DESCRIPTION OF THE FIGURES

FIG. 1 shows plots of the qRT-PCR Ct values for (A) 13629-L, (B) miR-150, (C) 13719-L, (D) 2548-L, (E) 14689-L, (F) miR-342-3p, (G) 13629-R, (H) IL18RAP-GUSB, and (I) 14621-L, in whole blood from healthy individuals, sepsis patients, and SIRS patients, as described in Example 1.

FIG. 2 shows an exemplary plot from FIG. 1, but including an indication of the median Ct for each condition (healthy, sepsis, or SIRS; heavy horizontal line), and a box delineating 25% above and 25% below the median, as described in Example 1. The shorter horizontal lines above and below the box indicate the data that was used to create the median and boxed data. Outliers were omitted.

FIG. 3 shows plots of the qRT-PCR Ct values for the combinations of (A) 13629-L−2548-L+14689-L; (B) 14689-L−miR-342-3p−13629-L−miR-150; (C) 13629-L−miR-150; (D) miR-150+14689-L−13629-L; (E) 13629-L+(IL18RAP−GUSB); (F) miR-150−13629-L+(IL18RAP−GUSB); (G) 13629-L+14621-L; (H) 13629-R+14621-L; (I) 13629-L+14621-L−miR-150; (J) 13629-R+14621-L−miR-150; and (K) 13629-L+14621-L−miR-150+(IL18RAP−GUSB), as described in Example 2.

FIG. 4 shows an exemplary plot from FIG. 3, but including an indication of the median Ct for each condition (healthy, sepsis, or SIRS; heavy horizontal line), and a box delineating 25% above and 25% below the median, as described in Example 2. The shorter horizontal lines above and below the box indicate the data that was used to create the median and boxed data. Outliers were omitted.

FIG. 5 shows Receiver Operator Characteristic (ROC) plots for sepsis versus healthy for the combinations of (A) 13629-L−2548-L+14689-L; (B) 14689-L−miR-342-3p−13629-L−miR-150; (C) 13629-L−miR-150; (D) miR-150+14689-L−13629-L; (E) 13629+(IL18RAP-GUSB); (F) miR-150−13629-L+(IL18RAP−GUSB); (G) 13629-L+14621-L; (H) 13629-R+14621-L; (I) 13629-L+14621-L−miR-150; (J) 13629-R+14621-L−miR-150; and (K) 13629-L+14621-L−miR-150+(IL18RAP−GUSB), as described in Example 3.

FIG. 6 shows ROC plots for sepsis versus SIRS for the combinations of (A) 13629-L−2548-L+14689-L; (B) 14689-L−miR-342-3p−13629-L−miR-150; (C) 13629-L−miR-150; (D) miR-150+14689-L−13629-L; (E) 13629+(IL18RAP-GUSB); (F) miR-150−13629-L+(IL18RAP-GUSB); (G) 13629-L+14621-L; (H) 13629-R+14621-L; (I) 13629-L+14621-L−miR-150; (J) 13629-R+14621-L−miR-150; and (K) 13629-L+14621-L−miR-150+(IL18RAP−GUSB), as described in Example 3.

FIG. 7 shows a plot of 13629-L−miR-150, and an analysis of the data using Tukey's HSD test, as described in Example 4.

FIG. 8 shows a plot of a panel comprising 13629-L and miR-150 in various patient populations and healthy individuals, as described in Example 5.

FIG. 9 shows Ct values for 13629-L and 13629-R in various patient populations and healthy individuals, as described in Example 5.

FIG. 10 shows a plot of a panel comprising 13629-L, 13629-R, and miR-150 in various patient populations and healthy individuals, as described in Example 5.

FIG. 11 shows a hypothetical model of the involvement of miR-150 and 13629 in IL-18 expression and NF-κB activation, for example, in sepsis, as discussed in Example 4.

### 4. DETAILED DESCRIPTION

### 4.1. Detecting Sepsis

### 4.1.1. General Methods

Methods of detecting sepsis by measuring levels of microRNA species are provided. Elevated levels of certain microRNA species are indicative of sepsis, and reduced levels of certain microRNA species are indicative of sepsis. In some embodiments, the method comprises detecting the level of at least one RNA selected from 13629, IL18-RAP, 13719, 2548, 14689, miR-150, and miR-342. In some embodiments, the method comprises detecting an above-normal level of at least one RNA selected from 13629, IL18RAP, and 13719 and/or a below-normal level of at least one RNA selected from 2548, 14689, miR-150, and miR-342. In some embodiments, the method comprises detecting an above-normal level of at least one RNA selected from 13629, IL18RAP, and 13719 and/or a below-normal level of at least two or at least three RNAs selected from 2548, 14689, miR-150, and miR-342. In some embodiments, the method comprises detecting the level of at least one RNA in blood.

In some embodiments, a method comprises detecting 13629, 2548, and 14689. In some such embodiments, an above-normal level of 13629 and/or a below-normal level of 2548 and/or 14689 is indicative of sepsis. In some embodiments, a method comprises detecting 13629, 14689, and miR-150. In some such embodiments, an above-normal level of 13629 and/or a below-normal level of 14689 and/or miR-150 is indicative of sepsis. In some embodiments, a method comprises detecting 13629, miR-342, miR-150, and 14689. In some such embodiments, an above-normal level of 13629 and/or a below-normal level of miR-342, miR-150, and/or 14689 is indicative of sepsis. In some embodiments, a method comprises detecting 13629 and miR-150. In some such embodiments, an above-normal level of 13629 and/or a below-normal level of miR-150 is indicative of sepsis. In some embodiments, a method comprises detecting 13629, IL18RAP, and miR-150. In some such embodiments, an above-normal level of 13629 and/or IL18RAP, and/or a below-normal level of miR-150 is indicative of sepsis. In some embodiments, a method comprises detecting 13629 and IL18RAP. In some such embodiments, an above-normal level of 13629 and/or IL18RAP is indicative of sepsis.

In some embodiments, the method further comprises detecting an above-normal level of at least one additional target RNA and/or a below-normal level of at least one additional target RNA. In some embodiments, a method comprises detecting both mature microRNA and pre-microRNA. In some embodiments, a method comprises detecting mature microRNA. In some embodiments, a method comprises detecting an mRNA, such as IL18RAP, and a microRNA. The mRNA and the microRNA may be detected in the same reaction or in separate reactions.

In some embodiments, a method described herein is able to distinguish between sepsis (i.e., SIRS with an infection) and SIRS (i.e., without an infection). In some such embodiments, a method described herein is able to distinguish between sepsis and SIRS with at least 80% sensitivity and/or at least 80% specificity. In some embodiments, a method described herein is able to distinguish between sepsis and SIRS with at least 80% sensitivity and/or at least 90% specificity. Further, in some embodiments, a method described herein is able to distinguish between sepsis and a healthy individual with at least 95% sensitivity and/or at least 95% specificity.

As used herein, the term “13629” includes pre-13629, mature 13629 (such as 13629-L), 13629-R, 13629-L and -R isomirs, and any other RNAs formed through processing of the pre-13629. Mature 13629-L has the sequence:

(SEQ ID NO: 1)

5′-TCTGATCAGGCAAAATTGCAGA-3′.

Pre-13629, which is the pre-microRNA form of 13629, has a sequence selected from:

(SEQ ID NO: 2)

5′-GCTCTGTGATTGCCTCTGATCAGGCAAAATTGCAGACTGTCTTCC
CAAATAGCCTGCAACTTTGCCTGATCAGAGGCAGTCACAGAGC-3′;
and

(SEQ ID NO: 3)

5′-GTGATTGCCTCTGATCAGGCAAAATTGCAGACTGTCTTCCCAAAT
AGCCTGCAACTTTGCCTGATCAGAGGCAGTCAC-3′.

An exemplary pre-13629 has the following structure, in which the mature 13629-L sequence is shown in bold.

```
                                              a a cuu
gugauugccucugaucaggcaaa uugcag cugu c
||||||||||||||||||||||| |||||| ||||
cacugacggagacuaguccguuu aacguc gaua c
                                              c c aac
```

13629-R forms are derived from the strand opposite 13629-L on the pre-13629. An exemplary 13629-R has the sequence:

(SEQ ID NO: 4)

5′-CCTGCAACTTTGCCTGATCAGA-3′.

Another exemplary 13629-L has the sequence:

(SEQ ID NO: 5)

5′-TGATCAGGCAAAATTGCAGACT-3′.

The 13629-L isomir represented by SEQ ID NO: 5 is deposited in MirBase as miR-4772-5p. It was found, however, that 13629-L having the sequence of SEQ ID NO: 1 was more abundant in certain sepsis samples than the miR-4772-5p sequence. As demonstrated in the Examples, at least mature 13629-L and mature 13629-R were detected at elevated levels in certain sepsis patients, using, e.g., quantitative RT-PCT.

As used herein, the term “13719” includes pre-13719, mature 13719 (such as 13719-L), 13719-R, mature 13719-L and -R isomirs, and any other RNAs formed through processing of the pre-13719. Mature 13719-L has the sequence:

(SEQ ID NO: 6)

5′-AGCTCTAGAAAGATTGTTGACC-3′.

Pre-13719, which is the pre-microRNA form of 13719, has the sequence:

(SEQ ID NO: 7)

5′-GGTTAGCACAGAGTGGGAGCTCTAGAAAGATTGTTGACCAATCAT
CTTATTGACTAGACCATCTTTCTAGAGTATAACTATTTTGGACACC-3′.

The pre-13719 has the following structure, in which the mature 13719-L sequence is shown in bold.

```
5′ TAG A GA T GAC CA
    GGT C CAGAGTGG GCTCTAGAAAGAT GTT CAAT T
    CCA G GTTTTATC TGAGATCTTTCTA CAG GTTA C
3′ CA AATA C ATCA TTC
```

13719-R forms are derived from the strand opposite 13719-L on the pre-13719, such as:

(SEQ ID NO: 8)

5′-TAGACCATCTTTCTAGAGTAT-3′.

Other exemplary 13719 sequences include:

(SEQ ID NO: 9)

5′-AGCTCTAGAAAGATTGTTGACCA-3′;

(SEQ ID NO: 10)

5′-AGCTCTAGAAAGATTGTTGAC-3′;
and

(SEQ ID NO: 11)

5′-AGCTCTAGAAAGATTGTTGA-3′.

As demonstrated in the Examples, at least mature 13719-L was detected at elevated levels in certain sepsis patients, using, e.g., quantitative RT-PCT.

As used herein, the term “miR-150” includes pre-miR-150, mature miR-150, miR-150*, miR-150 and miR-150*isomirs, and any other RNAs formed through processing of the pre-miR-150. Mature miR-150 has the sequence:

(SEQ ID NO: 12)

5′-TCTCCCAACCCTTGTACCAGTG-3′.

Pre-miR-150, which is the pre-microRNA form of miR-150, has the sequence:

(SEQ ID NO: 13)

5′-CTCCCCATGGCCCTGTCTCCCAACCCTTGTACCAGTGCTGGG
CTCAGACCCTGGTACAGGCCTGGGGGACAGGGACCTGGGGAC-3′.

The pre-miR-150 has the following structure, in which the mature miR-150 sequence is shown in bold.

```
c u - ac u u - g
  ucccca gg cccugucuccca ccu guaccag g cug g
  |||||| || |||||||||||| ||| ||||||| | |||
  aggggu cc gggacagggggu gga caugguc c gac c
c - a cc - c a u
```

MiR-150*forms are derived from the strand opposite the mature miR-150 on the pre-miR-150, such as:

(SEQ ID NO: 14)

5′-CTGGTACAGGCCTGGGGGACAG-3′.

Other exemplary miR-150 RNAs have the sequences:

(SEQ ID NO: 15)

5′-TCTCCCAACCCTTGTACCAGTGC-3′;

(SEQ ID NO: 16)

5′-TCTCCCAACCCTTGTACCAGT-3′;

(SEQ ID NO: 17)

5′-TCTCCCAACCCTTGTACCAG-3′;

(SEQ ID NO: 18)

5′-TCTCCCAACCCTTGTACCA-3′;
and

(SEQ ID NO: 19)

5′-TCTCCCAACCCTTGTACCAGTGA-3′.

As demonstrated in the Examples, at least mature miR-150 was detected at reduced levels in certain sepsis patients using, e.g., quantitative RT-PCR.

As used herein, the term “2548” includes pre-2548, mature 2548 (such as 2548-L), 2548-R, 2548-L and -R isomirs, and any other RNAs formed through processing of the pre-2548. Mature miR-2548-L has the sequence:

(SEQ ID NO: 20)

5′-CAACGGAAUCCCAAAAGCAGCU-3′.

Pre-2548, which is the pre-microRNA form of 2548, has the sequence:

(SEQ ID NO: 21)

5′-CGGCUGGACAGCGGGCAACGGAAUCCCAAAAGCAGCUGUUGUCUCCA
GAGCAUUCCAGCUGCGCUUGGAUUUCGUCCCCUGCUCUCCUGCCU-3′.

The pre-2548 has the following structure, in which the mature 2548-L sequence is shown in bold.

```
c u c ca c aa uu - c
  ggc gga agcggg acggaaucc aa gcagcug gu cu c
  ||| ||| |||||| ||||||||| || ||||||| || ||
  ccg ccu ucgucc ugcuuuagg uu cgucgac ua ga a
u u c cc - cg cu c g
```

2548-R forms are derived from the strand opposite 2548-L on the pre-2548, such as:

(SEQ ID NO: 22)

5′-GCUGCGCUUGGAUUUCGUCCCC-3′.

Other exemplary 2548 RNAs have the sequences:

(SEQ ID NO: 23)

5′-CAACGGAAUCCCAAAAGCAGCUG-3′;
and

(SEQ ID NO: 24)

5′-CAACGGAAUCCCAAAAGCAGCUGU-3′.

The 2548-L isomir represented by SEQ ID NO: 23 is deposited in MirBase as miR-191. It was found, however, that 2548-L having the sequence of SEQ ID NO: 20 was more abundant in certain sepsis samples than the miR-191 sequence. As demonstrated in the Examples, at least mature 2548-L was detected at reduced levels in certain sepsis patients, using, e.g., quantitative RT-PCT.

As used herein, the term “14689” includes pre-14689, mature 14689 (such as 14689-L), 14689-R, 14689-L and -R isomirs, and any other RNAs formed through processing of the pre-14689. Mature miR-14689-L has the sequence:

(SEQ ID NO: 25)

5′-UGCCCUGCCUGUUUUCUCCUUU-3′.

Pre-14689, which is the pre-microRNA form of 14689, has the sequence:

(SEQ ID NO: 26)

5′-UCCCUGCCCUGCCUGUUUUCUCCUUUGUGAUUUUAUGAGAACAA
AGGAGGAAAUAGGCAGGCCAGGGA-3′.

The pre-14689 has the following structure, in which the mature 14689-L sequence is shown in bold.

```
            c ga u
ucccug ccugccuguuuucuccuuugu uu u
|||||| ||||||||||||||||||||| ||
agggac ggacggauaaaggaggaaaca ag a
            c ag u
```

14689-R forms are derived from the strand opposite 14689-L on the pre-14689, such as:

(SEQ ID NO: 27)

5′-AAAGGAGGAAAUAGGCAGGCCA-3′.

Other exemplary 14689 RNAs have the sequences:

(SEQ ID NO: 28)

5′-TGCCCTGCCTGTTTTCTCCTTTGT-3′;

(SEQ ID NO: 29)

5′-TGCCCTGCCTGTTTTCTCCTTTG-3′;
and

(SEQ ID NO: 30)

5′-TGCCCTGCCTGTTTTCTCCTT-3′.

14689-L represented by SEQ ID NO: 25 is deposited in MirBase as miR-3173-5p. As demonstrated in the Examples, at least mature 14689-L was detected at reduced levels in certain sepsis patients, using, e.g., quantitative RT-PCT.

As used herein, the term “miR-342” includes pre-miR-342, mature miR-342-3p, mature miR-342-3p isomirs, miR-342-5p, miR-342-5p isomirs, and any other RNAs formed through processing of the pre-miR-342. Mature miR-342-3p has the sequence:

(SEQ ID NO: 31)

5′-UCUCACACAGAAAUCGCACCCGU-3′.

Pre-miR-342, which is the pre-microRNA form of miR-342-3p, has the sequence:

(SEQ ID NO: 32)

5′-GAAACUGGGCUCAAGGUGAGGGGUGCUAUCUGUGAUUGAGGGACAUG
GUUAAUGGAAUUGUCUCACACAGAAAUCGCACCCGUCACCUUGGCCUACU
UA-3′.

The pre-miR-342 has the following structure, in which the mature miR-342-3p sequence is shown in bold.

```
gaaac u g --ua auuga ugg a
          ugggc caagguga gggugc ucugug gggaca uu a
          ||||| |||||||| |||||| |||||| |||||| ||
          auccg guuccacu cccacg agacac cucugu ag u
-auuc - g cuaa ----a -ua g
```

MiR-342-5p forms are derived from the strand opposite the mature miR-342-3p on the pre-miR-342, such as:

(SEQ ID NO: 33)

5′-AGGGGUGCUAUCUGUGAUUGA-3′.

Other exemplary miR-342 RNAs have the sequences:

(SEQ ID NO: 34)

5′-TCTCACACAGAAATCGCACCCGTC-3′;
and

(SEQ ID NO: 35)

5′-TCTCACACAGAAATCGCACCCG-3′.

As demonstrated in the Examples, at least mature miR-342-3p was detected at reduced levels in certain sepsis patients, using, e.g., quantitative RT-PCR.

As used herein, the term “14621” includes pre-14621, mature 14621, 14621*, 14621 and 14621*isomirs, and any other RNAs formed through processing of the pre-14621. Mature 14621 (which is also referred to herein as “14621-L”) has the sequence:

(SEQ ID NO: 53)

5′-ACCCCACTCCTGGTACCA-3′.

Another exemplary mature 14621 has the sequence:

(SEQ ID NO: 54)

5′-ACCCCACTCCTGGTACC-3′.

The mature 14621 isomir represented by SEQ ID NO: 54 is deposited in MirBase as miR-4286. Thus, an exemplary pre-14621 sequence is the precursor of miR-4286 deposited in MirBase having the precursor sequence:

(SEQ ID NO: 56)

5′TACTTATGGCACCCCACTCCTGGTACCATAGTCATAAGTTAGGAGATG
TTAGAGCTGTGAGTACCATGACTTAAGTGTGGTGGCTTAAACATG 3′

The pre-miR-4286 has the following structure, in which the mature miR-4286 sequence is shown in bold:

```
--uac u g c ----- c - ca - a
          uua g cacc cac uc ugguac cauagu uaa guu g
          ||| | |||| ||| || |||||| |||||| ||| |||
          aau c gugg gug ag accaug gugucg auu uag g
guaca u g u aauuc u a ag g a
```

Another possible source of 14621 is a tRNA having a microRNA-like function: tRNA Leu TAA is located on chromosome 6 strand (+1) 144537684-144537766 (with last nucleotides CCA added after transcription). The sequence of this tRNA with 14621 shown bold is:

(SEQ ID NO: 57)

5′ACCAGGATGGCCGAGTGGTTAAGGCGTTGGACTTAAGATCCAATGGAC
ATATGTCCGCGTGGGTTCGAACCCCACTCCTGGTACCA 3′.

As demonstrated in the Examples, at least mature 14621 was detected at elevated levels in certain sepsis patients, using, e.g., quantitative RT-PCT.

As used herein, the term “IL18RAP” refers to human IL18RAP mRNA. In some embodiments, IL18RAP mRNA has the sequence of SEQ ID NO: 36:

5′-CTCTCTGGAT AGGAAGAAAT ATAGTAGAAC CCTTTGAAAA
TGGATATTTT CACATATTTT CGTTCAGATA CAAAAGCTGG
CAGTTACTGA AATAAGGACT TGAAGTTCCT TCCTCTTTTT
TTTATGTCTT AAGAGCAGGA AATAAAGAGA CAGCTGAAGG
TGTAGCCTTG ACCAACTGAA AGGGAAATCT TCATCCTCTG
AAAAAACATA TGTGATTCTC AAAAAACGCA TCTGGAAAAT
TGATAAAGAA GCGATTCTGT AGATTCTCCC AGCGCTGTTG
GGCTCTCAAT TCCTTCTGTG AAGGACAACA TATGGTGATG
GGGAAATCAG AAGCTTTGAG ACCCTCTACA CCTGGATATG
AATCCCCCTT CTAATACTTA CCAGAAATGA AGGGGATACT
CAGGGCAGAG TTCTGAATCT CAAAACACTC TACTCTGGCA
AAGGAATGAA GTTATTGGAG TGATGACAGG AACACGGGAG
AACAATGCTC TGTTTGGGCT GGATATTTCT TTGGCTTGTT
GCAGGAGAGC GAATTAAAGG ATTTAATATT TCAGGTTGTT
CCACAAAAAA ACTCCTTTGG ACATATTCTA CAAGGAGTGA
AGAGGAATTT GTCTTATTTT GTGATTTACC AGAGCCACAG
AAATCACATT TCTGCCACAG AAATCGACTC TCACCAAAAC
AAGTCCCTGA GCACCTGCCC TTCATGGGTA GTAACGACCT
ATCTGATGTC CAATGGTACC AACAACCTTC GAATGGAGAT
CCATTAGAGG ACATTAGGAA AAGCTATCCT CACATCATTC
AGGACAAATG TACCCTTCAC TTTTTGACCC CAGGGGTGAA
TAATTCTGGG TCATATATTT GTAGACCCAA GATGATTAAG
AGCCCCTATG ATGTAGCCTG TTGTGTCAAG ATGATTTTAG
AAGTTAAGCC CCAGACAAAT GCATCCTGTG AGTATTCCGC
ATCACATAAG CAAGACCTAC TTCTTGGGAG CACTGGCTCT
ATTTCTTGCC CCAGTCTCAG CTGCCAAAGT GATGCACAAA
GTCCAGCGGT AACCTGGTAC AAGAATGGAA AACTCCTCTC
TGTGGAAAGG AGCAACCGAA TCGTAGTGGA TGAAGTTTAT
GACTATCACC AGGGCACATA TGTATGTGAT TACACTCAGT
CGGATACTGT GAGTTCGTGG ACAGTCAGAG CTGTTGTTCA
AGTGAGAACC ATTGTGGGAG ACACTAAACT CAAACCAGAT
ATTCTGGATC CTGTCGAGGA CACACTGGAA GTAGAACTTG
GAAAGCCTTT AACTATTAGC TGCAAAGCAC GATTTGGCTT
TGAAAGGGTC TTTAACCCTG TCATAAAATG GTACATCAAA
GATTCTGACC TAGAGTGGGA AGTCTCAGTA CCTGAGGCGA
AAAGTATTAA ATCCACTTTA AAGGATGAAA TCATTGAGCG
TAATATCATC TTGGAAAAAG TCACTCAGCG TGATCTTCGC
AGGAAGTTTG TTTGCTTTGT CCAGAACTCC ATTGGAAACA
CAACCCAGTC CGTCCAACTG AAAGAAAAGA GAGGAGTGGT
GCTCCTGTAC ATCCTGCTTG GCACCATCGG GACCCTGGTG
GCCGTGCTGG CGGCGAGTGC CCTCCTCTAC AGGCACTGGA
TTGAAATAGT GCTGCTGTAC CGGACCTACC AGAGCAAGGA
TCAGACGCTT GGGGATAAAA AGGATTTTGA TGCTTTCGTA
TCCTATGCAA AATGGAGCTC TTTTCCAAGT GAGGCCACTT
CATCTCTGAG TGAAGAACAC TTGGCCCTGA GCCTATTTCC
TGATGTTTTA GAAAACAAAT ATGGATATAG CCTGTGTTTG
CTTGAAAGAG ATGTGGCTCC AGGAGGAGTG TATGCAGAAG
ACATTGTGAG CATTATTAAG AGAAGCAGAA GAGGAATATT
TATCTTGAGC CCCAACTATG TCAATGGACC CAGTATCTTT
GAACTACAAG CAGCAGTGAA TCTTGCCTTG GATGATCAAA
CACTGAAACT CATTTTAATT AAGTTCTGTT ACTTCCAAGA
GCCAGAGTCT CTACCTCATC TCGTGAAAAA AGCTCTCAGG
GTTTTGCCCA CAGTTACTTG GAGAGGCTTA AAATCAGTTC
CTCCCAATTC TAGGTTCTGG GCCAAAATGC GCTACCACAT
GCCTGTGAAA AACTCTCAGG GATTCACGTG GAACCAGCTC
AGAATTACCT CTAGGATTTT TCAGTGGAAA GGACTCAGTA
GAACAGAAAC CACTGGGAGG AGCTCCCAGC CTAAGGAATG
GTGAAATGAG CCCTGGAGCC CCCTCCAGTC CAGTCCCTGG
GATAGAGATG TTGCTGGACA GAACTCACAG CTCTGTGTGT
GTGTGTTCAG GCTGATAGGA AATTCAAAGA GTCTCCTGCC
AGCACCAAGC AAGCTTGATG GACAATGGAG TGGGATTGAG
ACTGTGGTTT AGAGCCTTTG ATTTCCTGGA CTGGACTGAC
GGCGAGTGAA TTCTCTAGAC CTTGGGTACT TTCAGTACAC
AACACCCCTA AGATTTCCCA GTGGTCCGAG CAGAATCAGA
AAATACAGCT ACTTCTGCCT TATGGCTAGG GAACTGTCAT
GTCTACCATG TATTGTACAT ATGACTTTAT GTATACTTGC
AATCAAATAA ATATTATTTT ATTAGAAA-3′

IL18RAP mRNA of SEQ ID NO: 36 has the following features within the mRNA:

| Feature | Bases |
|---|---|
| Exon 1 | 1-147 |
| Exon 2 | 148-384 |
| Exon 3 | 385-554 |
| Exon 4 | 555-879 |
| Exon 5 | 880-1063 |
| Exon 6 | 1064-1214 |
| Exon 7 | 1215-1280 |
| Exon 8 | 1281-1404 |
| Exon 9 | 1405-1556 |
| Exon 10 | 1557-1694 |
| Exon 11 | 1695-1868 |
| Exon 12 | 1869-2668 |
| Coding sequence | 485-2284 |
| Mature peptide coding sequence | 527-2281 |

As demonstrated in the Examples, IL18RAP was detected at elevated levels in certain sepsis patients, using, e.g., quantitative RT-PCR.

In the present disclosure, “a sequence selected from” encompasses both “one sequence selected from” and “one or more sequences selected from.” Thus, when “a sequence selected from” is used, it is to be understood that one, or more than one, of the listed sequences may be chosen.

As used here, a list such as “at least one RNA selected from 13629, 13719, miR-150, 2548, 14689, and miR-342” is intended to encompass “at least one RNA selected from: at least one 13629, at least one 13719, at least one miR-150, at least one 2548, at least one 14689, and at least one miR-342,” where 13629, 13719, miR-150, 2548, 14689, and miR-342 are defined as above. In other words, the at least one RNA may include more than one form of a listed RNA, but need not include at least one form of every listed RNA. Thus, in some embodiments, at least one RNA selected from 13629, 13719, miR-150, 2548, 14689, and miR-342 may include, for example, 13629-L and 13629-R; or may include, for example, 13629-L and 13629-R, 13719-L, and pre-miR-150, etc. Further, a list such as “at least two RNAs selected from 13629, 13719, miR-150, 2548, 14689, and miR-342” is intended to encompass at least one RNA from at least two types of RNAs in the list. In other words, “at least two RNAs selected from 13629, 13719, miR-150, 2548, 14689, and miR-342” may include, for example, a set containing 13629-L, 13629-R, and 2548-L; or a set containing 13629-L, 13629-R, 13719-L, pre-13719, and 2548-L; but does not include a set containing just 13629-L and 13629-R.

As used herein, language such as “detection of at least one RNA selected from IL18RAP and 13629” is intended to encompass detection of at least one portion of IL18RAP and/or detection of at least one 13629. Detection of at least one portion of IL18RAP includes detection of more than one portion of IL18RAP, such as, for example, detection of two separate portions of IL18RAP using two different sets of primers in either the same or separate RT-PCR reactions. In some embodiments, a set of primers used to detect IL18RAP hybridize to two different exons, such that IL18RAP mRNA can be distinguished from IL18RAP pre-mRNA and the IL18RAP gene. In some such embodiments, a set of primers for detecting IL18RAP spans exons 5 and 6. In other words, in some embodiments a set of primers for detecting IL18RAP comprises a first primer that anneals to exon 5 and a second primer that anneals to exon 6 such that the set of primers is capable of amplifying a nucleic acid comprising a portion of exon 5 and a portion of exon 6 in the presence of suitable reagents.

In the present disclosure, the term “target RNA” is used for convenience to refer to 13629, IL18RAP, 13719, miR-150, 2548, 14689, 14621, and/or miR-342, and also to other target RNAs. Thus, it is to be understood that when a discussion is presented in terms of a target RNA, that discussion is specifically intended to encompass 13629, IL18RAP, 13719, miR-150, 2548, 14689, 14621, and/or miR-342, and/or other target RNAs.

In some embodiments, detection of a level of certain target RNAs, such as 13629, IL18RAP, and/or 13719 that is greater than a normal level of target RNA indicates the presence of sepsis in a patient. In some embodiments, detection of a level of certain target RNAs, such as 2548, 14689, miR-150, and/or miR-342, that is lower than a normal level of target RNA indicates the presence of sepsis in a patient. In some embodiments, the detecting is done quantitatively. In other embodiments, the detecting is done qualitatively. In some embodiments, detecting a target RNA comprises forming a complex comprising a polynucleotide and a nucleic acid selected from a target RNA, a DNA amplicon of a target RNA, and a complement of a target RNA. In some embodiments, the level of the complex is then detected and compared to a normal level of the same complex.

“Sepsis” is an infection accompanied by an acute inflammatory reaction (systemic inflammatory response syndrome, or SIRS) with systemic manifestations associated with release of endogenous mediators of inflammation into the bloodstream. If left untreated, sepsis can become severe sepsis, which is often accompanied by the failure of at least one organ or septic shock, which is severe sepsis accompanied by organ hypoperfusion and hypotension that are poorly responsive to initial fluid resuscitation. The systemic inflammatory response may be mediated by toll-like receptors (“TLRs”).

Mature human microRNAs are typically composed of 17-27 contiguous ribonucleotides, and often are 21 or 22 nucleotides in length. While not intending to be bound by theory, mammalian microRNAs mature as described herein. A gene coding for a microRNA is transcribed, leading to production of a microRNA precursor known as the “pri-microRNA” or “pri-miRNA.” The pri-miRNA can be part of a polycistronic RNA comprising multiple pri-miRNAs. In some circumstances, the pri-miRNA forms a hairpin with a stem and loop, which may comprise mismatched bases. The hairpin structure of the pri-miRNA is recognized by Drosha, which is an RNase III endonuclease protein. Drosha can recognize terminal loops in the pri-miRNA and cleave approximately two helical turns into the stem to produce a 60-70 nucleotide precursor known as the “pre-microRNA” or “pre-miRNA.” Drosha can cleave the pri-miRNA with a staggered cut typical of RNase III endonucleases yielding a pre-miRNA stem loop with a 5′ phosphate and an approximately 2-nucleotide 3′ overhang. Approximately one helical turn of the stem (about 10 nucleotides) extending beyond the Drosha cleavage site can be essential for efficient processing. The pre-miRNA is subsequently actively transported from the nucleus to the cytoplasm by Ran-GTP and the export receptor Exportin-5.

The pre-miRNA can be recognized by Dicer, another RNase III endonuclease. In some circumstances, Dicer recognizes the double-stranded stem of the pre-miRNA. Dicer may also recognize the 5′ phosphate and 3′ overhang at the base of the stem loop. Dicer may cleave off the terminal loop two helical turns away from the base of the stem loop leaving an additional 5′ phosphate and an approximately 2-nucleotide 3′ overhang. The resulting siRNA-like duplex, which may comprise mismatches, comprises the mature microRNA and a similar-sized fragment known as the microRNA*. The microRNA and microRNA* may be derived from opposing arms of the pri-miRNA and pre-miRNA. The mature microRNA is then loaded into the RNA-induced silencing complex (“RISC”), a ribonucleoprotein complex. In some cases, the microRNA* also has gene silencing or other activity.

Nonlimiting exemplary small cellular RNAs include, in addition to microRNAs, small nuclear RNAs, tRNAs, ribosomal RNAs, snoRNAs, piRNAs, siRNAs, and small RNAs formed by processing any of those RNAs. In some embodiments, a target RNA is a small cellular RNA.

In some embodiments, a target RNA, such as at least one RNA selected from 13629, IL18RAP, 13719, miR-150, 2548, 14689, 14621, and/or miR-342, can be measured in samples collected at one or more times from a patient to monitor for the presence or progression of sepsis in the patient. In some embodiments, a target RNA, such as at least one RNA selected from 13629, IL18RAP, 13719, miR-150, 2548, 14689, 14621, and/or miR-342, can be measured in samples collected at one or more times from a patient undergoing therapy to monitor the progress of the therapy. In some such embodiments, the effectiveness of the therapy may be determined by monitoring.

In some embodiments, the sample to be tested is a bodily fluid, such as blood, sputum, mucus, saliva, urine, semen, etc. In some embodiments, a sample to be tested is a blood sample. In some embodiments, the blood sample is whole blood. In some embodiments, the blood sample is a sample of blood cells. In some embodiments, the blood sample is plasma. In some embodiments, the blood sample is serum. In some embodiments, the blood sample is a sample of peripheral blood mononuclear cells (PBMCs).

The clinical sample to be tested is, in some embodiments, freshly obtained. In other embodiments, the sample is a fresh frozen specimen. In some embodiments, the sample is a tissue sample, such as a formalin-fixed paraffin embedded sample. In some embodiments, the sample is a liquid cytology sample.

In some embodiments, the methods described herein are used for early detection of sepsis in a patient. In some embodiments, the methods described herein are used for routine screening of patients in clinical settings, such as hospitals, to detect sepsis before overt symptoms are detected. In some embodiments, the methods described herein are used to screen patients at risk for developing sepsis, such as post-surgical patients, patients with infections (such as urinary tract infections, skin infections, bacteremia, etc.), patients in intensive care (including, but not limited to, patients in an ICU under respiratory assistance), transplantation patients, and immunocompromised patients. Such screening may be carried out at regular intervals, in some embodiments, or at times in which the risk is believed to be greater. In some embodiments, the methods described herein are used to detect and/or confirm sepsis in patients that show one or more symptoms of sepsis.

In some embodiments, the methods described herein can be used to assess the effectiveness of a treatment for sepsis in a patient. In some embodiments, target RNA levels, such as at least one RNA selected from 13629, IL18RAP, 13719, miR-150, 2548, 14689, 14621, and miR-342, are determined at various times during the treatment, and are compared to target RNA levels from an archival sample taken from the patient, before the manifestation of any signs of sepsis or before beginning treatment. Ideally, target RNA levels in the archival sample evidence no aberrant changes in target RNA levels. Thus, in such embodiments, the progress of treatment of an individual with sepsis can be assessed by comparison to a sample from the same individual when he was healthy or prior to beginning treatment.

In some embodiments, a method comprises detecting at least one RNA selected from 13629, IL18RAP, 13719, miR-150, 2548, 14689, 14621, and miR-342. In some embodiments, in combination with detecting at least one RNA selected from 13629, IL18RAP, 13719, miR-150, 2548, 14689, 14621, and miR-342, a method further comprises detecting at least one additional target RNA. Such additional target RNAs include, but are not limited to, other microRNAs, small cellular RNAs, and mRNAs.

In embodiments in which the method comprises detecting levels of at least two RNAs, such as at least two RNAs selected from 13629, IL18RAP, 13719, miR-150, 2548, 14689, 14621, and miR-342, the levels of a plurality of RNAs may be detected concurrently or simultaneously in the same assay reaction. In some embodiments, RNA levels are detected concurrently or simultaneously in separate assay reactions. In some embodiments, RNA levels are detected at different times, e.g., in serial assay reactions. In some embodiments, mRNA levels are detected in at least one first assay reaction, while microRNA levels are detected in at least one second assay reaction.

In some embodiments, a method comprises detecting 13629, 2548, and 14689 in the same assay reaction. In some embodiments, a method comprises detecting 13629, 2548, and 14689 in two or more separate assay reactions. In some embodiments, a method comprises detecting 13629, 14689, and miR-150 in the same assay reaction. In some embodiments, a method comprises detecting 13629, 14689, and miR-150 in two or more separate assay reactions. In some embodiments, a method comprises detecting 13629, miR-342, miR-150, and 14689 in the same assay reaction. In some embodiments, a method comprises detecting 13629, miR-342, miR-150, and 14689 in two or more separate assay reactions. In some embodiments, a method comprises detecting 13629 and miR-150 in the same assay reaction. In some embodiments, a method comprises detecting 13629 and miR-150 in separate assay reactions. In some embodiments, a method comprises detecting 13629, IL18RAP, and miR-150 in the same assay reaction. In some embodiments, a method comprises detecting 13629, IL18RAP, and miR-150 in two or more separate assay reactions.

In some embodiments, a method comprises detecting the level of at least one RNA selected from 2548, IL18RAP, 14689, 14621, and miR-342 in a sample (such as blood) from the subject, wherein detection of a level of 2548, 14689, and/or miR-342 that is less than a normal level of the respective RNA, and wherein detection of a level of IL18RAP and/or 14621 that is greater than a normal level of the respective RNA, indicates the presence of sepsis in the subject. In some embodiments, a method further comprises detecting the level of at least one RNA selected from 13629, 13719, and miR-150 in a sample from the subject, wherein detection of a level of 13629 or 13719 that is greater than a normal level of the respective RNA, indicates the presence of sepsis in the subject, and wherein detection of a level of miR-150 that is less than a normal levels of miR-150, indicates the presence of sepsis in the subject. In some embodiments, a method comprises detecting the level of at least one RNA selected from 2548, IL18RAP, 14689, 14621, and miR-342 in a sample from a subject and comparing the level of the at least one RNA in the sample to normal levels of the at least one RNA, wherein a level of 2548, 14689, and/or miR-342 in the sample that are lower than normal levels of the RNA, and wherein detection of a level of IL18RAP and/or 14621 that is greater than a normal level of the respective RNA, indicates the presence of sepsis in the subject. In some embodiments, a method further comprises detecting the level of at least one RNA selected from 13629, 13719, and miR-150 in a sample from a subject and comparing the level of the at least one RNA in the sample to normal levels of the at least one RNA, wherein detection of a level of 13629 or 13719 that is greater than a normal level of the respective RNA, indicates the presence of sepsis in the subject, and wherein detection of a level of miR-150 that is less than a normal levels of miR-150, indicates the presence of sepsis in the subject. In some embodiments, a method comprising detecting a combination described above is able to distinguish between sepsis and SIRS.

In some embodiments, a method of facilitating diagnosis of sepsis in a subject is provided. Such methods comprise detecting the level of at least one RNA selected from 13629, IL18RAP, 13719, miR-150, 2548, 14689, 14621, and miR-342 in a sample from the subject. In some embodiments, information concerning the level of at least one RNA selected from 13629, IL18RAP, 13719, miR-150, 2548, 14689, 14621, and miR-342 in the sample from the subject is communicated to a medical practitioner. A “medical practitioner,” as used herein, refers to an individual or entity that diagnoses and/or treats patients, such as a hospital, a clinic, a physician's office, a physician, a nurse, or an agent of any of the aforementioned entities and individuals. In some embodiments, detecting the level of at least one RNA selected from 13629, IL18RAP, 13719, miR-150, 2548, 14689, 14621, and miR-342 is carried out at a laboratory that has received the subject's sample from the medical practitioner or agent of the medical practitioner. The laboratory carries out the detection by any method, including those described herein, and then communicates the results to the medical practitioner. A result is “communicated,” as used herein, when it is provided by any means to the medical practitioner. In some embodiments, such communication may be oral or written, may be by telephone, in person, by e-mail, by mail or other courier, or may be made by directly depositing the information into, e.g., a database accessible by the medical practitioner, including databases not controlled by the medical practitioner. In some embodiments, the information is maintained in electronic form. In some embodiments, the information can be stored in a memory or other computer readable medium, such as RAM, ROM, EEPROM, flash memory, computer chips, digital video discs (DVD), compact discs (CDs), hard disk drives (HDD), magnetic tape, etc.

In some embodiments, methods of detecting the presence of sepsis are provided. In some embodiments, methods of diagnosing sepsis are provided. In some embodiments, the method comprises obtaining a sample from a subject and providing the sample to a laboratory for detection of levels of at least one RNA selected from 13629, IL18RAP, 13719, miR-150, 2548, 14689, 14621, and miR-342 in the sample. In some embodiments, the method further comprises receiving a communication from the laboratory that indicates the levels of at least one RNA selected from 13629, IL18RAP, 13719, miR-150, 2548, 14689, 14621, and miR-342 in the sample. In some embodiments, sepsis is present if the level of at least one RNA selected from 13629 and 13719 in the sample is greater than a normal level of the RNA in the sample. In some embodiments, sepsis is present if the level of at least one RNA selected from 2548, 14689, miR-150, and miR-342 in the sample is lower than a normal level of the at least one RNA in the sample. A “laboratory,” as used herein, is any facility that detects the level of at least one RNA selected from 13629, IL18RAP, 13719, miR-150, 2548, 14689, 14621, and miR-342 in a sample by any method, including the methods described herein, and communicates the level to a medical practitioner. In some embodiments, a laboratory is under the control of a medical practitioner. In some embodiments, a laboratory is not under the control of the medical practitioner. In some embodiments, a laboratory is located within a hospital, whether or not the laboratory is owned, or controlled by, the hospital.

When a laboratory communicates the level of at least one RNA selected from 13629, IL18RAP, 13719, miR-150, 2548, 14689, 14621, and miR-342 to a medical practitioner, in some embodiments, the laboratory communicates a numerical value representing the level of at least one RNA selected from 13629, IL18RAP, 13719, miR-150, 2548, 14689, 14621, and miR-342 in the sample, with or without providing a numerical value for a normal level. In some embodiments, the laboratory communicates the level of at least one RNA selected from 13629, IL18RAP, 13719, miR-150, 2548, 14689, 14621, and miR-342 by providing a qualitative value, such as “high,” “low,” “elevated,” “decreased,” etc.

As used herein, when a method relates to detecting sepsis, determining the presence of sepsis, and/or diagnosing sepsis, the method includes activities in which the steps of the method are carried out, but the result is negative for the presence of sepsis. That is, detecting, determining, and diagnosing sepsis include instances of carrying out the methods that result in either positive or negative results (e.g., whether IL18RAP, 14621, 13629, and/or 13719 levels are normal or greater than normal, or whether 2548, 14689, miR-342, and/or miR-150 levels are normal or less than normal).

As used herein, the term “subject” means a human. In some embodiments, the methods described herein may be used on samples from non-human animals.

In some embodiments, a subject has a cardiac condition. Nonlimiting exemplary cardiac conditions include myocardial infarction, congestive heart failure, ischaemic heart disease, stable angina, unstable angina, acute coronary syndrome, pulmonary embolism, infective endocarditis, atrial fibrillation, recent angioplasty, recent coronary artery stent placement, and recent coronary artery bypass graft surgery. In some embodiments, a recent event, such as recent angioplasty, recent coronary artery stent placement, or recent coronary artery bypass graft surgery, has occurred within the last month, within the last three weeks, within the last two weeks, or within the last week.

In some instances, it has been found that miR-150 levels are reduced and 13629-L levels are increased in subjects with cardiac conditions. Thus, in some embodiments, the present methods comprise detecting levels of 13629-R in cardiac patients. In some instances, it has been found that 13629-R levels are reduced in cardiac patients relative to healthy individuals, but are increased in sepsis patients relative to healthy individuals. Thus, by including detection of 13629-R in a method described herein, in some embodiments, cardiac conditions can be distinguished from sepsis in assays in which 13629-L and miR-150 are detected.

Any of the methods described herein may further comprise treating a subject for sepsis, for example, when certain RNA levels have indicated the presence of sepsis in a subject. Treatments for sepsis include, but are not limited to, administering one or more antibiotics, administering a vasopressor, administering fluids, and administering oxygen. In some embodiments, one or more antibiotics are administered to a subject after the RNA detection methods described herein have indicated the presence of sepsis. In some embodiments, at least one of the antibiotics is a broad spectrum antibiotic. Nonlimiting exemplary broad spectrum antibiotics include amoxicillin, imipenem, levofloxacin, gatifloxacin, moxifloxacin, and ampicillin.

The common, or coordinate, expression of target RNAs that are physically proximal to one another in the genome permits the informative use of such chromosome-proximal target RNAs in methods herein.

The coding sequence for 13629 is located on chromosome 2 (strand+1): 103048749-103048826 (chromosome 2q12.1). The coding sequence for 13719 is located on chromosome 12 in the 3′ UTR of interleukin-1 receptor-associated kinase 3 (IRAK3), chromosome 12 (strand+1): 66644854-66644944 (chromosome 12q14.3). The coding sequence for miR-150 is located on chromosome 19: 50004042-50004125 on the minus strand (chromosome 19q13.33). The coding sequence for 2548 is located on chromosome 3: 49058051-49058142 on the minus strand (chromosome 3p21.31). The coding sequence for 14689 is located on chromosome 14: 95604256-95604323 on the minus strand (chromosome 14q32.23). The coding sequence for miR-342 is located on chromosome 14: 100575992-100576090 (chromosome 14q32.2). The coding sequence for 13719 is located on chromosome 8: 10524488-10524580 (chromosome 8p23.1) and/or on chromosome 6: 144537684-144537766. In some embodiments, the level of expression of one or more target RNAs located within about 1 kilobase (kb), within about 2 kb, within about 5 kb, within about 10 kb, within about 20 kb, within about 30 kb, within about 40 kb, and even within about 50 kb of the chromosomal location of 13629, IL18RAP, 13719, miR-150, 2548, 14689, 14621, or miR-342 is detected in lieu of, or in addition to, measurement of expression of the respective target RNA in the methods described herein. See Baskerville, S. and Bartel D. P. (2005) RNA 11:241-247.

In some embodiments, the methods further comprise detecting in a sample the expression of at least one target RNA gene located in close proximity to chromosomal features, such as inflammation-associated genomic regions, fragile sites, and human papilloma virus integration sites.

In some embodiments, more than one RNA is detected simultaneously in a single reaction. In some embodiments, at least 2, at least 3, at least 5, or at least 10 RNAs are detected simultaneously in a single reaction. In some embodiments, all of the selected target RNAs are detected simultaneously in a single reaction.

### 4.1.2. Exemplary Controls

In some embodiments, a normal level (a “control”) of a target RNA, such as an RNA selected from 13629, IL18RAP, 13719, miR-150, 2548, 14689, 14621, and miR-342, can be determined as an average level or range that is characteristic of normal levels found in samples from healthy individuals, against which the level measured in the sample can be compared. The determined average or range of a target RNA in normal subjects can be used as a benchmark for detecting above-normal levels or below-normal levels of the target RNA that are indicative of sepsis. In some embodiments, normal levels of a target RNA can be determined using individual or pooled RNA-containing samples from one or more healthy individuals.

In some embodiments, determining a normal level of a target RNA, such as at least one RNA selected from 13629, IL18RAP, 13719, miR-150, 2548, 14689, 14621, and miR-342, comprises detecting a complex comprising a polynucleotide for detection hybridized to a nucleic acid selected from a target RNA, a DNA amplicon of the target RNA, and a complement of the target RNA. That is, in some embodiments, a normal level can be determined by detecting a DNA amplicon of the target RNA, or a complement of the target RNA rather than the target RNA itself. In some embodiments, a normal level of such a complex is determined and used as a control. The normal level of the complex, in some embodiments, correlates to the normal level of the target RNA. Thus, when a normal level of a target is discussed herein, that level can, in some embodiments, be determined by detecting such a complex.

In some embodiments, a control comprises RNA from a sample from a single healthy individual. In some embodiments, a control comprises RNA from blood, such as whole blood or serum, of a single individual. In some embodiments, a control comprises RNA from a pool of samples from multiple healthy individuals. In some embodiments, a control comprises RNA from a pool of blood, such as whole blood or serum, from multiple individuals. In some embodiments, a control comprises commercially-available human RNA. In some embodiments, a normal level or normal range has already been predetermined prior to testing a sample for an elevated level.

In some embodiments, the normal level of a target RNA, such as at least one RNA selected from 13629, IL18RAP, 13719, miR-150, 2548, 14689, 14621, and miR-342, can be determined from one or more continuous cell lines, typically cell lines previously shown to have levels of RNAs that approximate the levels in healthy individuals.

In some embodiments, a method comprises detecting the level of at least one RNA selected from 13629, IL18RAP, 13719, miR-150, 2548, 14689, 14621, and miR-342. In some embodiment, in addition to detecting the level of at least one RNA selected from miR-13629, IL18RAP, 13719, miR-150, 2548, 14689, 14621, and miR-342, a method comprises detecting the level of at least one additional target RNA. In some embodiments, a method further comprises detecting the level of at least one additional target RNA. In some embodiments, a method further comprises comparing the level of at least one RNA selected from 13629, IL18RAP, 13719, miR-150, 2548, 14689, 14621, and miR-342 to a normal level of the at least one RNA. In some embodiments, a method further comprises comparing the level of at least one target RNA to a control level of the at least one target RNA. A control level of a target RNA is, in some embodiments, the level of the target RNA in a normal cell. A control level of a target RNA is, in some embodiments, the level of the target RNA in whole blood or serum from a healthy individual. In some such embodiments, a control level may be referred to as a normal level.

In some embodiments, a greater level of at least one RNA selected from 13629, IL18RAP, and 13719 in a patient sample relative to the level of the at least one RNA in a normal sample indicates sepsis. In some embodiments, a lower level of at least one RNA selected from 2548, 14689, miR-150, and miR-342 in a patient sample relative to the level of the RNA in a normal sample indicates sepsis. In some embodiments, a greater level of at least one additional target RNA relative to the level of the at least one additional target RNA in a normal sample indicates sepsis. In some embodiments, a lower level of at least one additional target RNA relative to the level of the at least one additional target RNA in a normal sample indicates sepsis.

In some embodiments, the level of a target RNA, such as at least one RNA selected from 13629, IL18RAP, 13719, miR-150, 2548, 14689, 14621, and miR-342, is compared to a reference level, e.g., from a confirmed sepsis patient. In some such embodiments, a similar level of a target RNA relative to the reference sample indicates sepsis.

In some embodiments, a level of at least one target RNA selected from 13629, IL18RAP, and 13719 that is at least about two-fold greater than a normal level of the respective target RNA indicates the presence of sepsis. In some embodiments, a level of at least one target RNA selected from 13629, IL18RAP, and 13719 that is at least about two-fold greater than the level of the respective target RNA in a control sample indicates the presence of a sepsis. In various embodiments, a level of at least one target RNA selected from 13629, IL18RAP, and 13719 that is at least about 3-fold, at least about 4-fold, at least about 5-fold, at least about 6-fold, at least about 7-fold, at least about 8-fold, at least about 9-fold, or at least about 10-fold greater than the level of the respective target RNA in a control sample indicates the presence of sepsis. In various embodiments, a level of at least one target RNA selected from 13629, IL18RAP, and 13719 that is at least about 3-fold, at least about 4-fold, at least about 5-fold, at least about 6-fold, at least about 7-fold, at least about 8-fold, at least about 9-fold, or at least about 10-fold greater than a normal level of the respective target RNA indicates the presence of sepsis.

In some embodiments, a level of at least one target RNA selected from 2548, 14689, miR-150, and miR-342 that is at least about two-fold less than a normal level of the respective target RNA indicates the presence of sepsis. In some embodiments, a level of at least one target RNA selected from 2548, 14689, miR-150, and miR-342 that is at least about two-fold less than the level of the respective target RNA in a control sample indicates the presence of a sepsis. In various embodiments, a level of at least one target RNA selected from 2548, 14689, miR-150, and miR-342 that is at least about 3-fold, at least about 4-fold, at least about 5-fold, at least about 6-fold, at least about 7-fold, at least about 8-fold, at least about 9-fold, or at least about 10-fold less than the level of the respective target RNA in a control sample indicates the presence of sepsis. In various embodiments, a level of at least one target RNA selected from 2548, 14689, miR-150, and miR-342 that is at least about 3-fold, at least about 4-fold, at least about 5-fold, at least about 6-fold, at least about 7-fold, at least about 8-fold, at least about 9-fold, or at least about 10-fold less than a normal level of the respective target RNA indicates the presence of sepsis.

In some embodiments, an increased level of at least one RNA selected from 13629, IL18RAP, and 13719 in a sample is indicative of sepsis. In some embodiments, a decreased level of at least one target RNA selected from 2548, 14689, miR-150, and miR-342 in a sample is indicative of sepsis. In some embodiments, an increased level of at least one RNA selected from 13629, IL18RAP, and 13719 and a decreased level of at least one target RNA selected from 2548, 14689, miR-150, and miR-342 in a sample is indicative of sepsis. In some embodiments, an increased level of 13629 and decreased levels of 2548 and 14689 are indicative of sepsis. In some embodiments, an increased level of 13629 and decreased levels of miR-150 and 14689 are indicative of sepsis. In some embodiments, an increased level of 13629 and decreased levels of miR-150, miR-342, and 14689 are indicative of sepsis. In some embodiments, increased levels of 13629 and IL18RAP and a decreased level of miR-150 is indicative of sepsis. In some embodiments, increased levels of 13629 and IL18RAP is indicative of sepsis.

In some embodiments, a control level of a target RNA, such as at least one RNA selected from 13629, IL18RAP, 13719, miR-150, 2548, 14689, 14621, and miR-342, is determined contemporaneously, such as in the same assay or batch of assays, as the level of the target RNA in a sample. In some embodiments, a control level of a target RNA, such as at least one RNA selected from 13629, IL18RAP, 13719, miR-150, 2548, 14689, 14621, and miR-342, is not determined contemporaneously as the level of the target RNA in a sample. In some such embodiments, the control level has been determined previously.

In some embodiments, the level of a target RNA is not compared to a control level, for example, when it is known that the target RNA is present at very low levels, or not at all, in normal cells. In such embodiments, detection of a high level of the target RNA in a sample is indicative of sepsis. Similarly, in some embodiments, if a target RNA is present at high levels in normal cells, whole blood, and/or serum, the detection of a very low level in a sample is indicative of sepsis.

Fold differences in RNA levels can be calculated, in some embodiments, from the equation 2^{-ΔCT}, where ΔCT=mean CtRNA-A—mean CtRNA-B, and where “mean CtRNA-A” and “mean CtRNA-B” refer to the mean Ct for RNA A and RNA B, respectively. Ct refers to the threshold cycle for the RNA. RNA A and RNA B may, in some embodiments, be the same RNA, but in two different sample types, such as in healthy patient samples and SIRS patient samples. In other embodiments, RNA A and RNA B may be different RNAs measured in the same sample type, such as a first RNA from a sepsis patient sample and a second RNA from a sepsis patient sample. In some such embodiments, the two RNAs may both be markers of sepsis, or one of the RNAs may be a marker of sepsis and one of the RNAs may be one that is not expected to be present at different levels in sepsis (versus healthy). In some embodiments, the equation 2^{-ΔCT} represents a fold-change value between the two RNAs.

In some embodiments, the ratio of the levels of two RNAs is determined. For example, in some embodiments, the ratio of the levels of two RNAs selected from 13629, IL18RAP, 13719, miR-150, 2548, 14689, 14621, and miR-342 is determined. In some embodiments, the ratio of the levels of 13629 and IL18RAP is determined. In some embodiments, the ratio of the levels of 13629 and 13719, miR-150, 2548, 14689, 14621, or miR-342 is determined. In some embodiments, the ratio of the levels of 13719 and 13629, miR-150, 2548, 14689, 14621, or miR-342 is determined. In some embodiments, the ratio of the levels of miR-150 and 13629, 13719, 2548, 14689, 14621, or miR-342 is determined. In some embodiments, the ratio of the levels of 2548 and 13629, IL18RAP, 13719, miR-150, 14689, 14621, or miR-342 is determined. In some embodiments, the ratio of the levels of 14689 and 13629, IL18RAP, 13719, miR-150, 2548, 14621, or miR-342 is determined. In some embodiments, the ratio of the levels of 14621 and 13629, IL18RAP, 13719, miR-150, 2548, 14689, or miR-342 is determined. In some embodiments, the ratio of the levels of miR-342 and 13629, IL18RAP, 13719, miR-150, 2548, 14689, or 14621 is determined. By comparing the ratios of RNAs, in some instances, results obtained in different experiments, different reactions, and/or on different machines, for example, can be compared to one another. In some embodiments when a normalizing control is not used, a ratio of the levels of two RNAs selected from 13629, IL18RAP, 13719, miR-150, 2548, 14689, 14621, and miR-342 may be used, for example, to allow more accurate cross-comparison between experiments. In some embodiments, however, such a ratio is not necessary for accurate cross-comparison between experiments.

In some embodiments, a ratio of the levels of more than two RNAs is determined. For example, in some embodiments, a ratio of the levels of more than two RNAs selected from 13629, IL18RAP, 13719, miR-150, 2548, 14689, 14621, and miR-342 is determined. As a nonlimiting example, a ratio of the levels of three RNAs may be determined by taking the ratio of (RNA-A+RNA-B) to RNA-C. Similarly, as a further nonlimiting example, the ratio of RNA-A to (RNA-B−RNA-C) may be determined. Similar ratios may be constructed using the levels of four, five, six, seven, etc., RNAs.

In some embodiments, the levels of RNAs selected from 13629, IL18RAP, 13719, miR-150, 2548, 14689, 14621, and miR-342 as determined by qRT-PCR are used in a ratio. In some embodiments, the levels of RNAs selected from 13629, IL18RAP, 13719, miR-150, 2548, 14689, 14621, and miR-342 as determined by microarray are used in a ratio. Levels of RNAs determined by other methods may also be used in a ratio.

### 4.1.3. Exemplary Methods of Preparing RNAs

Target RNA can be prepared by any appropriate method. Total RNA can be isolated by any method, including, but not limited to, the protocols set forth in Wilkinson, M. (1988) Nucl. Acids Res. 16(22):10,933; and Wilkinson, M. (1988) Nucl. Acids Res. 16(22): 10934, or by using commercially-available kits or reagents, such as the TRIzol® reagent (Invitrogen™), Total RNA Extraction Kit (iNtRON Biotechnology), Total RNA Purification Kit (Norgen Biotek Corp.), RNAqueous™ (Ambion), MagMAX™ (Ambion), RecoverAll™ (Ambion), RNeasy (Qiagen), etc.

In some embodiments, small RNAs are isolated or enriched. In some embodiments “small RNA” refers to RNA molecules smaller than about 200 nucleotides (nt) in length. In some embodiments, “small RNA” refers to RNA molecules smaller than about 100 nt, smaller than about 90 nt, smaller than about 80 nt, smaller than about 70 nt, smaller than about 60 nt, smaller than about 50 nt, or smaller than about 40 nt.

Enrichment of small RNAs can be accomplished by method. Such methods include, but are not limited to, methods involving organic extraction followed by adsorption of nucleic acid molecules on a glass fiber filter using specialized binding and wash solutions, and methods using spin column purification. Enrichment of small RNAs may be accomplished using commercially-available kits, such as mirVana™ Isolation Kit (Applied Biosystems), mirPremier™ microRNA Isolation Kit (Sigma-Aldrich), PureLink™ miRNA Isolation Kit (Invitrogen), miRCURY™ RNA isolation kit (Exiqon), microRNA Purification Kit (Norgen Biotek Corp.), miRNeasy kit (Qiagen), etc. In some embodiments, purification can be accomplished by the TRIzol® (Invitrogen) method, which employs a phenol/isothiocyanate solution to which chloroform is added to separate the RNA-containing aqueous phase. Small RNAs are subsequently recovered from the aqueous by precipitation with isopropyl alcohol. In some embodiments, small RNAs can be purified using chromatographic methods, such as gel electrophoresis using the flashPAGE™ Fractionator available from Applied Biosystems.

In some embodiments, small RNA is isolated from other RNA molecules to enrich for target RNAs, such that the small RNA fraction (e.g., containing RNA molecules that are 200 nucleotides or less in length, such as less than 100 nucleotides in length, such as less than 50 nucleotides in length, such as from about 10 to about 40 nucleotides in length) is substantially pure, meaning it is at least about 80%, 85%, 90%, 95% pure or more, but less than 100% pure, with respect to larger RNA molecules. Alternatively, enrichment of small RNA can be expressed in terms of fold-enrichment. In some embodiments, small RNA is enriched by about, at least about, or at most about 5×, 10×, 20×, 30×, 40×, 50×, 60×, 70×, 80×, 90×, 100×, 110×, 120×, 130×, 140×, 150×, 160×, 170×, 180×, 190×, 200×, 210×, 220×, 230×, 240×, 250×, 260×, 270×, 280×, 290×, 300×, 310×, 320×, 330×, 340×, 350×, 360×, 370×, 380×, 390×, 400×, 410×, 420×, 430×, 440×, 450×, 460×, 470×, 480×, 490×, 500×, 600×, 700×, 800×, 900×, 1000×, 1100×, 1200×, 1300×, 1400×, 1500×, 1600×, 1700×, 1800×, 1900×, 2000×, 3000×, 4000×, 5000×, 6000×, 7000×, 8000×, 9000×, 10,000× or more, or any range derivable therein, with respect to the concentration of larger RNAs in an RNA isolate or total RNA in a sample.

In some embodiments, RNA levels are measured in a sample in which RNA has not first been purified from the cells. In some embodiments, RNA levels are measured in a sample in which RNA has been isolated, but not enriched for small RNAs.

In some embodiments, RNA is modified before a target RNA is detected. In some embodiments, the modified RNA is total RNA. In other embodiments, the modified RNA is small RNA that has been purified from total RNA or from cell lysates, such as RNA less than 200 nucleotides in length, such as less than 100 nucleotides in length, such as less than 50 nucleotides in length, such as from about 10 to about 40 nucleotides in length. RNA modifications that can be utilized in the methods described herein include, but are not limited to, the addition of a poly-dA or a poly-dT tail, which can be accomplished chemically or enzymatically, and/or the addition of a small molecule, such as biotin.

In some embodiments, a target RNA, such as at least one RNA selected from 13629, IL18RAP, 13719, miR-150, 2548, 14689, 14621, and miR-342, is reverse transcribed. In some embodiments, cDNA is modified when it is reverse transcribed, such as by adding a poly-dA or a poly-dT tail during reverse transcription. In other embodiments, RNA is modified before it is reverse transcribed. In some embodiments, total RNA is reverse transcribed. In other embodiments, small RNAs are isolated or enriched before the RNA is reverse transcribed.

When a target RNA, such as at least one RNA selected from 13629, IL18RAP, 13719, miR-150, 2548, 14689, 14621, and miR-342, is reverse transcribed, a complement of the target RNA is formed. In some embodiments, the complement of a target RNA is detected rather than a target RNA itself (or a DNA copy thereof). Thus, when the methods discussed herein indicate that a target RNA is detected, or the level of a target RNA is determined, such detection or determination may be carried out on a complement of a target RNA instead of, or in addition to, the target RNA itself. In some embodiments, when the complement of a target RNA is detected rather than the target RNA, a polynucleotide for detection is used that is complementary to the complement of the target RNA. In such embodiments, a polynucleotide for detection comprises at least a portion that is identical in sequence to the target RNA, although it may contain thymidine in place of uridine, and/or comprise other modified nucleotides.

In some embodiments, the method of detecting a target RNA, such as at least one RNA selected from 13629, IL18RAP, 13719, miR-150, 2548, 14689, 14621, and miR-342, comprises amplifying cDNA complementary to the target RNA. Such amplification can be accomplished by any method. Exemplary methods include, but are not limited to, real time PCR, endpoint PCR, and amplification using T7 polymerase from a T7 promoter annealed to a cDNA, such as provided by the SenseAmp Plus™ Kit available at Implen, Germany.

When a target RNA or a cDNA complementary to a target RNA is amplified, in some embodiments, a DNA amplicon of the target RNA is formed. A DNA amplicon may be single stranded or double-stranded. In some embodiments, when a DNA amplicon is single-stranded, the sequence of the DNA amplicon is related to the target RNA in either the sense or antisense orientation. In some embodiments, a DNA amplicon of a target RNA is detected rather than the target RNA itself. Thus, when the methods discussed herein indicate that a target RNA is detected, or the level of a target RNA is determined, such detection or determination may be carried out on a DNA amplicon of the target RNA instead of, or in addition to, the target RNA itself. In some embodiments, when the DNA amplicon of the target RNA is detected rather than the target RNA, a polynucleotide for detection is used that is complementary to the complement of the target RNA. In some embodiments, when the DNA amplicon of the target RNA is detected rather than the target RNA, a polynucleotide for detection is used that is complementary to the target RNA. Further, in some embodiments, multiple polynucleotides for detection may be used, and some polynucleotides may be complementary to the target RNA and some polynucleotides may be complementary to the complement of the target RNA.

In some embodiments, the method of detecting one or more target RNAs, including 13629, IL18RAP, 13719, miR-150, 2548, 14689, 14621, and/or miR-342, comprises RT-PCR, as described below. In some embodiments, detecting one or more target RNAs comprises real-time monitoring of an RT-PCR reaction, which can be accomplished by any method. Such methods include, but are not limited to, the use of TaqMan®, Molecular beacon, or Scorpion probes (i.e., FRET probes) and the use of intercalating dyes, such as SYBR green, EvaGreen, thiazole orange, YO-PRO, TO-PRO, etc.

### 4.1.4. Exemplary Analytical Methods

As described above, methods are presented for detecting sepsis. In some embodiments, the method comprises detecting a level of at least one RNA selected from 13629, IL18RAP, 13719, miR-150, 2548, 14689, 14621, and miR-342. In some embodiments, the method further comprises detecting a level of at least one additional target RNA. In some embodiments, a method comprises detecting a level of at least one, at least two, or at least three RNAs selected from 2548, 14689, miR-150, and miR-342 in a sample from a subject. In some such embodiments, a level of 2548, 14689, miR-150, or miR-342 that is less than a normal level of the respective RNA, indicates the presence of sepsis in the subject. In some embodiments, a method further comprises detecting a level of at least one RNA or at least two RNAs selected from 13629, IL18RAP, and 13719 in a sample from a subject. In some such embodiments, a level of 13629, IL18RAP, or 13719 that is greater than a normal level of the respective RNA, indicates the presence of sepsis in the subject. In some embodiments, a method comprises detecting the levels of 13629, 2548, and 14689. In some embodiments, a method comprises detecting the levels of miR-150, 14689, and 13629. In some embodiments, a method comprises detecting the levels of 14689, miR-342, 13629, and miR-150. In some embodiments, a method comprises detecting the levels of 13629, IL18RAP, and miR-150. In some embodiments, a method comprises detecting the levels of IL18RAP and miR-150. In some embodiments, a method comprises detecting the levels of 13629 and 14621. In some embodiments, a method comprises detecting the levels of 13629, 14621, and miR-150. In some embodiments, a method comprises detecting the levels of 13629, 14621, and IL18RAP. In some embodiments, a method comprises detecting the levels of 13629, 14621, miR-150, and IL18RAP. In some embodiments, a target RNA is an mRNA. In some embodiments, a target RNA, in its mature form, comprises fewer than 30 nucleotides. In some embodiments, a target RNA is a microRNA. In some embodiments, a target RNA is a small cellular RNA.

In some embodiments, in addition to detecting a level of at least one RNA selected from 13629, IL18RAP, 13719, miR-150, 2548, 14689, 14621, and miR-342, a method further comprises detecting a level of at least one target RNA of the human miRNome. As used herein, the term “human miRNome” refers to all microRNA genes in a human cell and the mature microRNAs produced therefrom.

Any analytical procedure capable of permitting specific and quantifiable (or semi-quantifiable) detection of a target RNA, such as at least one RNA selected from 13629, IL18RAP, 13719, miR-150, 2548, 14689, 14621, and miR-342, may be used in the methods herein presented. Such analytical procedures include, but are not limited to, the microarray methods and the RT-PCR methods set forth in the Examples, and methods known to those skilled in the art.

In some embodiments, detection of a target RNA, such as at least one RNA selected from 13629, IL18RAP, 13719, miR-150, 2548, 14689, 14621, and miR-342, comprises forming a complex comprising a polynucleotide that is complementary to a target RNA or to a complement thereof, and a nucleic acid selected from the target RNA, a DNA amplicon of the target RNA, and a complement of the target RNA. Thus, in some embodiments, the polynucleotide forms a complex with a target RNA. In some embodiments, the polynucleotide forms a complex with a complement of the target RNA, such as a cDNA that has been reverse transcribed from the target RNA. In some embodiments, the polynucleotide forms a complex with a DNA amplicon of the target RNA. When a double-stranded DNA amplicon is part of a complex, as used herein, the complex may comprise one or both strands of the DNA amplicon. Thus, in some embodiments, a complex comprises only one strand of the DNA amplicon. In some embodiments, a complex is a triplex and comprises the polynucleotide and both strands of the DNA amplicon. In some embodiments, the complex is formed by hybridization between the polynucleotide and the target RNA, complement of the target RNA, or DNA amplicon of the target RNA. The polynucleotide, in some embodiments, is a primer or probe.

In some embodiments, a method comprises detecting the complex. In some embodiments, the complex does not have to be associated at the time of detection. That is, in some embodiments, a complex is formed, the complex is then dissociated or destroyed in some manner, and components from the complex are detected. An example of such a system is a TaqMan® assay. In some embodiments, when the polynucleotide is a primer, detection of the complex may comprise amplification of the target RNA, a complement of the target RNA, or a DNA amplicon of a target RNA.

In some embodiments the analytical method used for detecting at least one target RNA, including at least one RNA selected from 13629, IL18RAP, 13719, miR-150, 2548, 14689, 14621, and miR-342, in the methods set forth herein includes real-time quantitative RT-PCR. See Chen, C. et al. (2005) Nucl. Acids Res. 33:e179 and PCT Publication No. WO 2007/117256, which are incorporated herein by reference in its entirety. In some embodiments, the analytical method used for detecting at least one target RNA includes the method described in U.S. Publication No. US2009/0123912 A1, which is incorporated herein by reference in its entirety. In an exemplary method described in that publication, an extension primer comprising a first portion and second portion, wherein the first portion selectively hybridizes to the 3′ end of a particular RNA and the second portion comprises a sequence for universal primer, is used to reverse transcribe the RNA to make a cDNA. A reverse primer that selectively hybridizes to the 5′ end of the RNA and a universal primer are then used to amplify the cDNA in a quantitative PCR reaction.

In some embodiments, the analytical method used for detecting at least one target RNA, including at least one RNA selected from 13629, IL18RAP, 13719, miR-150, 2548, 14689, 14621, and miR-342, includes the use of a TaqMan® probe. In some embodiments, the analytical method used for detecting at least one target RNA includes a TaqMan® assay, such as the TaqMan® MicroRNA Assays sold by Applied Biosystems, Inc. In an exemplary TaqMan® assay, total RNA is isolated from the sample. In some embodiments, the assay can be used to analyze about 10 ng of total RNA input sample, such as about 9 ng of input sample, such as about 8 ng of input sample, such as about 7 ng of input sample, such as about 6 ng of input sample, such as about 5 ng of input sample, such as about 4 ng of input sample, such as about 3 ng of input sample, such as about 2 ng of input sample, and even as little as about 1 ng of input sample containing RNAs.

The TaqMan® assay utilizes a stem-loop primer that is specifically complementary to the 3′-end of a target RNA. In an exemplary TaqMan® assay, hybridizing the stem-loop primer to the target RNA is followed by reverse transcription of the target RNA template, resulting in extension of the 3′ end of the primer. The result of the reverse transcription is a chimeric (DNA) amplicon with the step-loop primer sequence at the 5′ end of the amplicon and the cDNA of the target RNA at the 3′ end. Quantitation of the target RNA is achieved by real time RT-PCR using a universal reverse primer having a sequence that is complementary to a sequence at the 5′ end of all stem-loop target RNA primers, a target RNA-specific forward primer, and a target RNA sequence-specific TaqMan® probe.

The assay uses fluorescence resonance energy transfer (“FRET”) to detect and quantitate the synthesized PCR product. Typically, the TaqMan® probe comprises a fluorescent dye molecule coupled to the 5′-end and a quencher molecule coupled to the 3′-end, such that the dye and the quencher are in close proximity, allowing the quencher to suppress the fluorescence signal of the dye via FRET. When the polymerase replicates the chimeric amplicon template to which the TaqMan® probe is bound, the 5′-nuclease of the polymerase cleaves the probe, decoupling the dye and the quencher so that FRET is abolished and a fluorescence signal is generated. Fluorescence increases with each RT-PCR cycle proportionally to the amount of probe that is cleaved.

Additional exemplary methods for RNA detection and/or quantification are described, e.g., in U.S. Publication No. US 2007/0077570 (Lao et al.), PCT Publication No. WO 2007/025281 (Tan et al.), U.S. Publication No. US2007/0054287 (Bloch), PCT Publication No. WO2006/0130761 (Bloch), and PCT Publication No. WO 2007/011903 (Lao et al.), which are incorporated by reference herein in their entireties for any purpose.

In some embodiments, quantitation of the results of real-time RT-PCR assays is done by constructing a standard curve from a nucleic acid of known concentration and then extrapolating quantitative information for target RNAs of unknown concentration. In some embodiments, the nucleic acid used for generating a standard curve is an RNA (e.g., an mRNA, or a microRNA or other small RNA) of known concentration. In some embodiments, the nucleic acid used for generating a standard curve is a purified double-stranded plasmid DNA or a single-stranded DNA generated in vitro.

In some embodiments, where the amplification efficiencies of the target nucleic acids and the endogenous reference are approximately equal, quantitation is accomplished by the comparative Ct (cycle threshold, e.g., the number of PCR cycles required for the fluorescence signal to rise above background) method. Ct values are inversely proportional to the amount of nucleic acid target in a sample. In some embodiments, Ct values of a target RNA, such as at least one RNA selected from 13629, IL18RAP, 13719, miR-150, 2548, 14689, 14621, and miR-342, can be compared with a control or calibrator, such as RNA (e.g., an mRNA, or a microRNA or other small RNA) from normal tissue. In some embodiments, the Ct values of the calibrator and the target RNA are normalized to an appropriate endogenous housekeeping gene. In some embodiments, the Ct values for one or more target RNAs are normalized to an appropriate endogenous housekeeping gene. In some such embodiments, the Ct value for an appropriate endogenous housekeeping gene is subtracted from the Ct value for a target RNA. In some embodiments, a threshold Ct (or a “cutoff Ct”) value for a target RNA, such as at least one RNA selected from 13629, IL18RAP, 13719, miR-150, 2548, 14689, 14621, and miR-342, below which sepsis is indicated, has previously been determined. In some embodiments, a threshold Ct (or a “cutoff Ct”) value for a target RNA, such as at least one RNA selected from 13629, IL18RAP, 13719, miR-150, 2548, 14689, 14621, and miR-342, above which sepsis is indicated, has previously been determined. In such embodiments, a control sample may not be assayed concurrently with the test sample.

In addition to the TaqMan® assays, other real-time RT-PCR chemistries useful for detecting and quantitating PCR products in the methods presented herein include, but are not limited to, Molecular Beacons, Scorpion probes and intercalating dyes, such as SYBR Green, EvaGreen, thiazole orange, YO-PRO, TO-PRO, etc., which are discussed below.

In some embodiments, real-time RT-PCR detection is performed specifically to detect and quantify the level of a single target RNA. The target RNA, in some embodiments, is an RNA selected from 13629, IL18RAP, 13719, miR-150, 2548, 14689, 14621, and miR-342.

As described above, in some embodiments, in addition to detecting the level of at least one RNA selected from 13629, IL18RAP, 13719, miR-150, 2548, 14689, 14621, and miR-342, the level of at least one additional target RNA is detected.

In various other embodiments, real-time RT-PCR detection is utilized to detect, in a single multiplex reaction, at least 2, at least 3, at least 4, at least 5, at least 6, at least 7, or at least 8 target RNAs, including at least one RNA selected from 13629, IL18RAP, 13719, miR-150, 2548, 14689, 14621, and miR-342.

In some multiplex embodiments, a plurality of probes, such as TaqMan® probes, each specific for a different RNA target, is used. In some embodiments, each target RNA-specific probe is spectrally distinguishable from the other probes used in the same multiplex reaction.

In some embodiments, quantitation of real-time RT PCR products is accomplished using a dye that binds to double-stranded DNA products, such as SYBR Green, EvaGreen, thiazole orange, YO-PRO, TO-PRO, etc. In some embodiments, the assay is the QuantiTect SYBR Green PCR assay from Qiagen. In this assay, total RNA is first isolated from a sample. Total RNA is subsequently poly-adenylated at the 3′-end and reverse transcribed using a universal primer with poly-dT at the 5′-end. In some embodiments, a single reverse transcription reaction is sufficient to assay multiple target RNAs. Real-time RT-PCR is then accomplished using target RNA-specific primers and an miScript Universal Primer, which comprises a poly-dT sequence at the 5′-end. SYBR Green dye binds non-specifically to double-stranded DNA and upon excitation, emits light. In some embodiments, buffer conditions that promote highly-specific annealing of primers to the PCR template (e.g., available in the QuantiTect SYBR Green PCR Kit from Qiagen) can be used to avoid the formation of non-specific DNA duplexes and primer dimers that will bind SYBR Green and negatively affect quantitation. Thus, as PCR product accumulates, the signal from SYBR Green increases, allowing quantitation of specific products.

Real-time RT-PCR is performed using any RT-PCR instrumentation available in the art. Typically, instrumentation used in real-time RT-PCR data collection and analysis comprises a thermal cycler, optics for fluorescence excitation and emission collection, and optionally a computer and data acquisition and analysis software.

In some embodiments, a target gene is detected using an automated sample handling and/or analysis platform. In some embodiments, commercially available automated analysis platforms are utilized. For example, in some embodiments, the GeneXpert® system (Cepheid, Sunnyvale, Calif.) is utilized.

The GeneXpert® utilizes a self-contained, single use cartridge. Sample extraction, amplification, and detection may all be carried out within this self-contained “laboratory in a cartridge.” (See e.g., U.S. Pat. Nos. 5,958,349, 6,403,037, 6,440,725, 6,783,736, 6,818,185; each of which is herein incorporated by reference in its entirety.) In some embodiments, the GeneXpert® system provides results of the detection methods described herein in less than three hours, less than two hours, less than 100 minutes, or less than 90 minutes, allowing for rapid detection and treatment of sepsis.

Components of the cartridge include, but are not limited to, processing chambers containing reagents, filters, and capture technologies useful to extract, purify, and amplify target nucleic acids. A valve enables fluid transfer from chamber to chamber and contains nucleic acids lysis and filtration components. An optical window enables real-time optical detection. A reaction tube enables very rapid thermal cycling.

In some embodiments, the GenXpert® system includes a plurality of modules for scalability. Each module includes a plurality of cartridges, along with sample handling and analysis components.

In some embodiments, after the sample is added to the cartridge, the sample is contacted with lysis buffer and released RNA is bound to an RNA-binding substrate such as a silica or glass substrate. The sample supernatant is then removed and the RNA eluted in an elution buffer such as a Tris/EDTA buffer. The eluate may then be processed in the cartridge to detect target RNAs as described herein. In some embodiments, the eluate is used to reconstitute at least some of the reverse transcription and/or amplification reagents, which are present in the cartridge as lyophilized particles.

In some embodiments, the analytical method used in the methods described herein is a DASL® (cDNA-mediated Annealing, Selection, Extension, and Ligation) Assay, such as the MicroRNA Expression Profiling Assay available from Illumina, Inc. (See http://www.illumina.com/downloads/MicroRNAAssayWorkflow.pdf). In some embodiments, total RNA is isolated from a sample to be analyzed by any method. Additionally, in some embodiments, small RNAs are isolated from a sample to be analyzed by any method. Total RNA or isolated small RNAs may then be polyadenylated (>18 A residues are added to the 3′-ends of the RNAs in the reaction mixture). The RNA is reverse transcribed using a biotin-labeled DNA primer that comprises from the 5′ to the 3′ end, a sequence that includes a PCR primer site and a poly-dT region that binds to the poly-dA tail of the sample RNA. The resulting biotinylated cDNA transcripts are then hybridized to a solid support via a biotin-streptavidin interaction and contacted with one or more target RNA-specific polynucleotides. The target RNA-specific polynucleotides comprise, from the 5′-end to the 3′-end, a region comprising a PCR primer site, region comprising an address sequence, and a target RNA-specific sequence.

In some DASL® embodiments, the target RNA-specific sequence comprises at least 8, at least 9, at least 10, at least 11, at least 12, at least 13, at least 14, at least 15, at least 16, at least 17, at least 18, at least 19 contiguous nucleotides having a sequence that is complementary to at least 8, at least 9, at least 10, at least 11, at least 12, at least 13, at least 14, at least 15, at least 16, at least 17, at least 18, at least 19 contiguous nucleotides of at least one RNA selected from 13629, IL18RAP, 13719, miR-150, 2548, 14689, 14621, and miR-342. In some DASL® embodiments, the target RNA-specific sequence comprises at least 8, at least 9, at least 10, at least 11, at least 12, at least 13, at least 14, at least 15, at least 16, at least 17, at least 18, at least 19, at least 20, at least 21, at least 22, at least 23, or at least 24 contiguous nucleotides having a sequence that is complementary to at least 8, at least 9, at least 10, at least 11, at least 12, at least 13, at least 14, at least 15, at least 16, at least 17, at least 18, at least 19, at least 20, at least 21, at least 22, at least 23, or at least 24 contiguous nucleotides of another target RNA.

After hybridization, the target RNA-specific polynucleotide is extended, and the extended products are then eluted from the immobilized cDNA array. A second PCR reaction using a fluorescently-labeled universal primer generates a fluorescently-labeled DNA comprising the target RNA-specific sequence. The labeled PCR products are then hybridized to a microbead array for detection and quantitation.

In some embodiments, the analytical method used for detecting and quantifying the levels of the at least one target RNA, including at least one RNA selected from 13629, IL18RAP, 13719, miR-150, 2548, 14689, 14621, and miR-342, in the methods described herein is a bead-based flow cytometric assay. See Lu J. et al. (2005) Nature 435:834-838, which is incorporated herein by reference in its entirety. An example of a bead-based flow cytometric assay is the xMAP® technology of Luminex, Inc. (See http://www.luminexcorp.com/technology/index.html). In some embodiments, total RNA is isolated from a sample and is then labeled with biotin. The labeled RNA is then hybridized to target RNA-specific capture probes (e.g., FlexmiR™ products sold by Luminex, Inc. at http://www.luminexcorp.com/products/assays/index.html) that are covalently bound to microbeads, each of which is labeled with 2 dyes having different fluorescence intensities. A streptavidin-bound reporter molecule (e.g., streptavidin-phycoerythrin, also known as “SAPE”) is attached to the captured target RNA and the unique signal of each bead is read using flow cytometry. In some embodiments, the RNA sample (total RNA or enriched small RNAs) is first polyadenylated, and is subsequently labeled with a biotinylated 3DNA™ dendrimer (i.e., a multiple-arm DNA with numerous biotin molecules bound thereto), such as those sold by Marligen Biosciences as the Vantage™ microRNA Labeling Kit, using a bridging polynucleotide that is complementary to the 3′-end of the poly-dA tail of the sample RNA and to the 5′-end of the polynucleotide attached to the biotinylated dendrimer. The streptavidin-bound reporter molecule is then attached to the biotinylated dendrimer before analysis by flow cytometry. See http://www.marligen.com/vantage-microrna-labeling-kit.html. In some embodiments, biotin-labeled RNA is first exposed to SAPE, and the RNA/SAPE complex is subsequently exposed to an anti-phycoerythrin antibody attached to a DNA dendrimer, which can be bound to as many as 900 biotin molecules. This allows multiple SAPE molecules to bind to the biotinylated dendrimer through the biotin-streptavidin interaction, thus increasing the signal from the assay.

In some embodiments, the analytical method used for detecting and quantifying the levels of the at least one target RNA, including at least one RNA selected from 13629, IL18RAP, 13719, miR-150, 2548, 14689, 14621, and miR-342, in the methods described herein is by gel electrophoresis and detection with labeled probes (e.g., probes labeled with a radioactive or chemiluminescent label), such as by Northern blotting. In some embodiments, total RNA is isolated from the sample, and then is size-separated by SDS polyacrylamide gel electrophoresis. The separated RNA is then blotted onto a membrane and hybridized to radiolabeled complementary probes. In some embodiments, exemplary probes contain one or more affinity-enhancing nucleotide analogs as discussed below, such as locked nucleic acid (“LNA”) analogs, which contain a bicyclic sugar moiety instead of deoxyribose or ribose sugars. See, e.g., Várallyay, E. et al. (2008) Nature Protocols 3(2):190-196, which is incorporated herein by reference in its entirety. In some embodiments, the total RNA sample can be further purified to enrich for small RNAs. In some embodiments, target RNAs can be amplified by, e.g., rolling circle amplification using a long probe that is complementary to both ends of a target RNA (“padlocked probes”), ligation to circularize the probe followed by rolling circle replication using the target RNA hybridized to the circularized probe as a primer. See, e.g., Jonstrup, S. P. et al. (2006) RNA 12:1-6, which is incorporated herein by reference in its entirety. The amplified product can then be detected and quantified using, e.g., gel electrophoresis and Northern blotting.

In alternative embodiments, labeled probes are hybridized to isolated total RNA in solution, after which the RNA is subjected to rapid ribonuclease digestion of single-stranded RNA, e.g., unhybridized portions of the probes or unhybridized target RNAs. In these embodiments, the ribonuclease treated sample is then analyzed by SDS-PAGE and detection of the radiolabeled probes by, e.g., Northern blotting. See mirVana™ miRNA Detection Kit sold by Applied Biosystems, Inc. product literature at http://www.ambion.com/catalog/CatNum.php?1552.

In some embodiments, the analytical method used for detecting and quantifying the at least one target RNA, including at least one RNA selected from 13629, IL18RAP, 13719, miR-150, 2548, 14689, 14621, and miR-342, in the methods described herein is by hybridization to a microarray. See, e.g., Liu, C. G. et al. (2004) Proc. Nat'l Acad. Sci. USA 101:9740-9744; Lim, L. P. et al. (2005) Nature 433:769-773, each of which is incorporated herein by reference in its entirety, and Example 1.

In some embodiments, detection and quantification of a target RNA using a microarray is accomplished by surface plasmon resonance. See, e.g., Nanotech News (2006), available at http://nano.cancer.gov/news_center/nanotech_news_{—}2006-10-30b.asp. In these embodiments, total RNA is isolated from a sample being tested. Optionally, the RNA sample is further purified to enrich the population of small RNAs. After purification, the RNA sample is bound to an addressable microarray containing probes at defined locations on the microarray.

Any capture probe suitable for use in detecting a selected target RNA, such as at least one RNA selected from 13629, IL18RAP, 13719, miR-150, 2548, 14689, 14621, and miR-342, may be used. Nonlimiting exemplary capture probes comprise a region comprising a sequence selected from:

(SEQ ID NO: 37)

5′-TCTGCAATTTTGCCTGATCAGA-3′ for 13629;

(SEQ ID NO: 38)

5′-AGTCTGCAATTTTGCCTGATCA-3′ for 13629;

(SEQ ID NO: 39)

5′-GGTCAACAATCTTTCTAGAGCT-3′ for 13719;

(SEQ ID NO: 40)

5′-GTCAACAATCTTTCTAGAGCT-3′ for 13719;

(SEQ ID NO: 41)

5′-CACTGGTACAAGGGTTGGGAGA-3′ for miR-150;

(SEQ ID NO: 42)

5′-AGCTGCTTTTGGGATTCCGTTG-3′ for 2548;

(SEQ ID NO: 43)

5′-CAGCTGCTTTTGGGATTCCGTTG-3′ for 2548;

(SEQ ID NO: 44)

5′-AAAGGAGAAAACAGGCAGGGCA-3′ for 14689;

(SEQ ID NO: 45)

5′-ACGGGTGCGATTTCTGTGTGAGA-3′ for miR-342;
and

(SEQ ID NO: 55)

5′-TGGTACCAGGAGTGGGGT-3′ for 14621.

Further nonlimiting exemplary probes comprise a region having at least 8, at least 9, at least 10, at least 11, at least 12, at least 13, at least 14, at least 15, at least 16, at least 17, or at least 18 contiguous nucleotides of a sequence selected from SEQ ID NOs: 37 to 45 and 55. A probe may further comprise at least a second region that does not comprise a sequence that is identical to at least 8 contiguous nucleotides of a sequence selected from SEQ ID NOs: 37 to 45 and 55.

Nonlimiting exemplary probes comprise a region having at least 8, at least 9, at least 10, at least 11, at least 12, at least 13, at least 14, at least 15, at least 16, at least 17, or at least 18, at least 19, at least 20, at least 25, at least 30, at least 40, at least 50, at least 60, or at least 70 contiguous nucleotides of a sequence selected from:

(SEQ ID NO: 46)

5′-GCTCTGTGACTGCCTCTGATCAGGCAAAGTTGCAGGCTATTTGGGAA
GACAGTCTGCAATTTTGCCTGATCAGAGGCAATCACAGAGC-3′
for 13629;

(SEQ ID NO: 47)

5′-GTGACTGCCTCTGATCAGGCAAAGTTGCAGGCTATTTGGGAAGACAG
TCTGCAATTTTGCCTGATCAGAGGCAATCAC-3′ for 13629;

(SEQ ID NO: 48)

5′-GGTGTCCAAAATAGTTATACTCTAGAAAGATGGTCTAGTCAATAAGA
TGATTGGTCAACAATCTTTCTAGAGCTCCCACTCTGTGCTAACC-3′
for 13719;

(SEQ ID NO: 49)

5′-GTCCCCAGGTCCCTGTCCCCCAGGCCTGTACCAGGGTCTGAGCCCA
GCACTGGTACAAGGGTTGGGAGACAGGGCCATGGGGAG-3′
for miR-150;

(SEQ ID NO: 50)

5′-AGGCAGGAGAGCAGGGGACGAAATCCAAGCGCAGCTGGAATGCTC
TGGAGACAACAGCTGCTTTTGGGATTCCGTTGCCCGCTGTCCAGCCG-3′
for 2548;

(SEQ ID NO: 51)

5′-TCCCTGGCCTGCCTATTTCCTCCTTTGTTCTCATAAAATCACAAAGG
AGAAAACAGGCAGGGCAGGGA-3′ for 14689;

(SEQ ID NO: 52)

5′-TAAGTAGGCCAAGGTGACGGGTGCGATTTCTGTGTGAGACAATTCCA
TTAACCATGTCCCTCAATCACAGATAGCACCCCTCACCTTGAGCCCAGTT
TC-3′ for miR-342;

(SEQ ID NO: 58)

5′-CATGTTTAAGCCACCACACTTAAGTCATGGTACTCACAGCTCTAACA
TCTCCTAACTTATGACTATGGTACCAGGAGTGGGGTGCCATAAGTA-3′
for 14621;
and

(SEQ ID NO: 59)

5′-TGGTACCAGGAGTGGGGTTCGAACCCACGCGGACATATGTCCATTGG
ATCTTAAGTCCAACGCCTTAACCACTCGGCCATCCTGGT-3′
for 14621.

In some embodiments, the probes contain one or more affinity-enhancing nucleotide analogs as discussed below, such as locked nucleic acid (“LNA”) nucleotide analogs. After hybridization to the microarray, the RNA that is hybridized to the array is first polyadenylated, and the array is then exposed to gold particles having poly-dT bound to them. The amount of bound target RNA is quantitated using surface plasmon resonance.

In some embodiments, microarrays are utilized in a RNA-primed, Array-based Klenow Enzyme (“RAKE”) assay. See Nelson, P. T. et al. (2004) Nature Methods 1(2):1-7; Nelson, P. T. et al. (2006) RNA 12(2):1-5, each of which is incorporated herein by reference in its entirety. In some embodiments, total RNA is isolated from a sample. In some embodiments, small RNAs are isolated from a sample. The RNA sample is then hybridized to DNA probes immobilized at the 5′-end on an addressable array. The DNA probes comprise, in some embodiments, from the 5′-end to the 3′-end, a first region comprising a “spacer” sequence which is the same for all probes, a second region comprising three thymidine-containing nucleosides, and a third region comprising a sequence that is complementary to a target RNA of interest, such as at least one RNA selected from 13629, IL18RAP, 13719, miR-150, 2548, 14689, 14621, and miR-342.

After the sample is hybridized to the array, it is exposed to exonuclease I to digest any unhybridized probes. The Klenow fragment of DNA polymerase I is then applied along with biotinylated dATP, allowing the hybridized target RNAs to act as primers for the enzyme with the DNA probe as template. The slide is then washed and a streptavidin-conjugated fluorophore is applied to detect and quantitate the spots on the array containing hybridized and Klenow-extended target RNAs from the sample.

In some embodiments, the RNA sample is reverse transcribed. In some embodiments, the RNA sample is reverse transcribed using a biotin/poly-dA random octamer primer. When than primer is used, the RNA template is digested and the biotin-containing cDNA is hybridized to an addressable microarray with bound probes that permit specific detection of target RNAs. In typical embodiments, the microarray includes at least one probe comprising at least 8, at least 9, at least 10, at least 11, at least 12, at least 13, at least 14, at least 15, at least 16, at least 17, at least 18, at least 19, at least 20, at least 21, at least 22, at least 23, or at least 24 contiguous nucleotides identically present in, or complementary to a region of, a target RNA, such as at least one RNA selected from 13629, IL18RAP, 13719, miR-150, 2548, 14689, 14621, and miR-342. After hybridization of the cDNA to the microarray, the microarray is exposed to a streptavidin-bound detectable marker, such as a fluorescent dye, and the bound cDNA is detected. See Liu C. G. et al. (2008) Methods 44:22-30, which is incorporated herein by reference in its entirety.

In some embodiments, target RNAs, including 13629, IL18RAP, 13719, miR-150, 2548, 14689, 14621, and miR-342, are detected and quantified in an ELISA-like assay using probes bound in the wells of microtiter plates. See Mora J. R. and Getts R. C. (2006) BioTechniques 41:420-424 and supplementary material in BioTechniques 41(4):1-5; U.S. Patent Publication No. 2006/0094025 to Getts et al., each of which is incorporated by reference herein in its entirety. In some embodiments, a sample of RNA is either polyadenylated, or is reverse transcribed and the cDNA is polyadenylated. The RNA or cDNA is hybridized to probes immobilized in the wells of a microtiter plates, wherein each of the probes comprises a sequence that is identically present in, or complementary to a region of, a target RNA, such as at least one RNA selected from 13629, IL18RAP, 13719, miR-150, 2548, 14689, 14621, and miR-342. In some embodiments, the hybridized RNAs are labeled using a capture sequence, such as a DNA dendrimer (such as those available from Genisphere, Inc., http://www.genisphere.com/about_{—}3 dna.html) that is labeled with a plurality of biotin molecules or with a plurality of horseradish peroxidase molecules, and a bridging polynucleotide that contains a poly-dT sequence at the 5′-end that binds to the poly-dA tail of the captured nucleic acid, and a sequence at the 3′-end that is complementary to a region of the capture sequence. If the capture sequence is biotinylated, the microarray is then exposed to streptavidin-bound horseradish peroxidase. Hybridization of target RNAs is detected by the addition of a horseradish peroxidase substrate such as tetramethylbenzidine (TMB) and measurement of the absorbance of the solution at 450 nM.

In still other embodiments, an addressable microarray is used to detect a target RNA using quantum dots. See Liang, R. Q. et al. (2005) Nucl. Acids Res. 33(2):e17, available at http://www.pubmedcentral.nih.gov/articlerender.fcgi?artid=548377, which is incorporated herein by reference in its entirety. In some embodiments, total RNA is isolated from a sample. In some embodiments, small RNAs are isolated from the sample. The 3′-ends of the target RNAs are biotinylated using biotin-X-hydrazide. The biotinylated target RNAs are captured on a microarray comprising immobilized probes comprising sequences that are identically present in, or complementary to a region of, target RNAs, including 13629, IL18RAP, 13719, miR-150, 2548, 14689, 14621, and miR-342. The hybridized target RNAs are then labeled with quantum dots via a biotin-streptavidin binding. A confocal laser causes the quantum dots to fluoresce and the signal can be quantified. In alternative embodiments, RNAs can be detected using a colorimetric assay. In these embodiments, RNAs are labeled with streptavidin-conjugated gold followed by silver enhancement. The gold nanoparticules bound to the hybridized target RNAs catalyze the reduction of silver ions to metallic silver, which can then be detected colorimetrically with a CCD camera

In some embodiments, detection and quantification of one or more target RNAs is accomplished using microfluidic devices and single-molecule detection. In some embodiments, target RNAs in a sample of isolated total RNA are hybridized to two probes, one which is complementary to nucleic acids at the 5′-end of the target RNA and the second which is complementary to the 3′-end of the target RNA. Each probe comprises, in some embodiments, one or more affinity-enhancing nucleotide analogs, such as LNA nucleotide analogs and each is labeled with a different fluorescent dye having different fluorescence emission spectra. The sample is then flowed through a microfluidic capillary in which multiple lasers excite the fluorescent probes, such that a unique coincident burst of photons identifies a particular target RNA, and the number of particular unique coincident bursts of photons can be counted to quantify the amount of the target RNA in the sample. See U.S. Patent Publication No. 2006/0292616 to Neely et al., which is hereby incorporated by reference in its entirety. In some alternative embodiments, a target RNA-specific probe can be labeled with 3 or more distinct labels selected from, e.g., fluorophores, electron spin labels, etc., and then hybridized to an RNA sample, such as total RNA, or a sample that is enriched in small RNAs. Nonlimiting exemplary target RNA-specific probes include probes comprising sequences selected from SEQ ID NOs: 37 to 52, 55, 58, and 59; sequences having at least 8, at least 9, at least 10, at least 11, at least 12, at least 13, at least 14, at least 15, at least 16, at least 17, or at least 18 contiguous nucleotides of a sequence selected from SEQ ID NOs: 37 to 45 and 55; and sequences having at least 8, at least 9, at least 10, at least 11, at least 12, at least 13, at least 14, at least 15, at least 16, at least 17, at least 18, at least 19, at least 20, at least 25, at least 30, at least 40, at least 50, at least 60, or at least 70 contiguous nucleotides of a sequence selected from SEQ ID NOs: 46 to 52, 58, and 59.

Optionally, the sample RNA is modified before hybridization. The target RNA/probe duplex is then passed through channels in a microfluidic device and that comprise detectors that record the unique signal of the 3 labels. In this way, individual molecules are detected by their unique signal and counted. See U.S. Pat. Nos. 7,402,422 and 7,351,538 to Fuchs et al., U.S. Genomics, Inc., each of which is incorporated herein by reference in its entirety.

In some embodiments, the detection and quantification of one or more target RNAs is accomplished by a solution-based assay, such as a modified Invader assay. See Allawi H. T. et al. (2004) RNA 10:1153-1161, which is incorporated herein by reference in its entirety. In some embodiments, the modified invader assay can be performed on unfractionated detergent lysates of cervical cells. In other embodiments, the modified invader assay can be performed on total RNA isolated from cells or on a sample enriched in small RNAs. The target RNAs in a sample are annealed to two probes which form hairpin structures. A first probe has a hairpin structure at the 5′ end and a region at the 3′-end that has a sequence that is complementary to the sequence of a region at the 5′-end of a target RNA. The 3′-end of the first probe is the “invasive polynucleotide”. A second probe has, from the 5′ end to the 3′-end a first “flap” region that is not complementary to the target RNA, a second region that has a sequence that is complementary to the 3′-end of the target RNA, and a third region that forms a hairpin structure. When the two probes are bound to a target RNA target, they create an overlapping configuration of the probes on the target RNA template, which is recognized by the Cleavase enzyme, which releases the flap of the second probe into solution. The flap region then binds to a complementary region at the 3′-end of a secondary reaction template (“SRT”). A FRET polynucleotide (having a fluorescent dye bound to the 5′-end and a quencher that quenches the dye bound closer to the 3′ end) binds to a complementary region at the 5′-end of the SRT, with the result that an overlapping configuration of the 3′-end of the flap and the 5′-end of the FRET polynucleotide is created. Cleavase recognizes the overlapping configuration and cleaves the 5′-end of the FRET polynucleotide, generates a fluorescent signal when the dye is released into solution.

### 4.1.5. Exemplary Polynucleotides

In some embodiments, polynucleotides are provided. In some embodiments, synthetic polynucleotides are provided. Synthetic polynucleotides, as used herein, refer to polynucleotides that have been synthesized in vitro either chemically or enzymatically. Chemical synthesis of polynucleotides includes, but is not limited to, synthesis using polynucleotide synthesizers, such as OligoPilot (GE Healthcare), ABI 3900 DNA Synthesizer (Applied Biosystems), and the like. Enzymatic synthesis includes, but is not limited, to producing polynucleotides by enzymatic amplification, e.g., PCR.

In some embodiments, a polynucleotide is provided that comprises at least 8, at least 9, at least 10, at least 11, at least 12, at least 13, at least 14, at least 15, at least 16, at least 17, or at least 18 contiguous nucleotides of a sequence selected from SEQ ID NOs: 37 to 45 and 55. In some embodiments, a polynucleotide is provided that comprises at least 8, at least 9, at least 10, at least 11, at least 12, at least 13, at least 14, at least 15, at least 16, at least 17, at least 18, at least 19, at least 20, at least 25, at least 30, at least 40, at least 50, at least 60, or at least 70 contiguous nucleotides of a sequence selected from SEQ ID NOs: 46 to 52, 58, and 59.

In various embodiments, a polynucleotide comprises fewer than 500, fewer than 300, fewer than 200, fewer than 150, fewer than 100, fewer than 75, fewer than 50, fewer than 40, or fewer than 30 nucleotides. In various embodiments, a polynucleotide is between 8 and 200, between 8 and 150, between 8 and 100, between 8 and 75, between 8 and 50, between 8 and 40, or between 8 and 30 nucleotides long.

In some embodiments, the polynucleotide is a primer. In some embodiments, the primer is labeled with a detectable moiety. In some embodiments, a primer is not labeled. A primer, as used herein, is a polynucleotide that is capable of specifically hybridizing to a target RNA or to a cDNA reverse transcribed from the target RNA or to an amplicon that has been amplified from a target RNA or a cDNA (collectively referred to as “template”), and, in the presence of the template, a polymerase and suitable buffers and reagents, can be extended to form a primer extension product.

In some embodiments, the polynucleotide is a probe. In some embodiments, the probe is labeled with a detectable moiety. A detectable moiety, as used herein, includes both directly detectable moieties, such as fluorescent dyes, and indirectly detectable moieties, such as members of binding pairs. When the detectable moiety is a member of a binding pair, in some embodiments, the probe can be detectable by incubating the probe with a detectable label bound to the second member of the binding pair. In some embodiments, a probe is not labeled, such as when a probe is a capture probe, e.g., on a microarray or bead. In some embodiments, a probe is not extendable, e.g., by a polymerase. In other embodiments, a probe is extendable.

In some embodiments, the polynucleotide is a FRET probe that in some embodiments is labeled at the 5′-end with a fluorescent dye (donor) and at the 3′-end with a quencher (acceptor), a chemical group that absorbs (i.e., suppresses) fluorescence emission from the dye when the groups are in close proximity (i.e., attached to the same probe). In other embodiments, the donor and acceptor are not at the ends of the FRET probe. Thus, in some embodiments, the emission spectrum of the donor moiety should overlap considerably with the absorption spectrum of the acceptor moiety.

### 4.1.5.1. Exemplary Polynucleotide Modifications

In some embodiments, the methods of detecting at least one target RNA described herein employ one or more polynucleotides that have been modified, such as polynucleotides comprising one or more affinity-enhancing nucleotide analogs. Modified polynucleotides useful in the methods described herein include primers for reverse transcription, PCR amplification primers, and probes. In some embodiments, the incorporation of affinity-enhancing nucleotides increases the binding affinity and specificity of a polynucleotide for its target nucleic acid as compared to polynucleotides that contain only deoxyribonucleotides, and allows for the use of shorter polynucleotides or for shorter regions of complementarity between the polynucleotide and the target nucleic acid.

In some embodiments, affinity-enhancing nucleotide analogs include nucleotides comprising one or more base modifications, sugar modifications and/or backbone modifications.

In some embodiments, modified bases for use in affinity-enhancing nucleotide analogs include 5-methylcytosine, isocytosine, pseudoisocytosine, 5-bromouracil, 5-propynyluracil, 6-aminopurine, 2-aminopurine, inosine, diaminopurine, 2-chloro-6-aminopurine, xanthine and hypoxanthine.

In some embodiments, affinity-enhancing nucleotide analogs include nucleotides having modified sugars such as 2′-substituted sugars, such as 2′-O-alkyl-ribose sugars, 2′-amino-deoxyribose sugars, 2′-fluoro-deoxyribose sugars, 2′-fluoro-arabinose sugars, and 2′-O-methoxyethyl-ribose (2′MOE) sugars. In some embodiments, modified sugars are arabinose sugars, or d-arabino-hexitol sugars.

In some embodiments, affinity-enhancing nucleotide analogs include backbone modifications such as the use of peptide nucleic acids (PNA; e.g., an oligomer including nucleobases linked together by an amino acid backbone). Other backbone modifications include phosphorothioate linkages, phosphodiester modified nucleic acids, combinations of phosphodiester and phosphorothioate nucleic acid, methylphosphonate, alkylphosphonates, phosphate esters, alkylphosphonothioates, phosphoramidates, carbamates, carbonates, phosphate triesters, acetamidates, carboxymethyl esters, methylphosphorothioate, phosphorodithioate, p-ethoxy, and combinations thereof.

In some embodiments, a polynucleotide includes at least one affinity-enhancing nucleotide analog that has a modified base, at least nucleotide (which may be the same nucleotide) that has a modified sugar, and/or at least one internucleotide linkage that is non-naturally occurring.

In some embodiments, an affinity-enhancing nucleotide analog contains a locked nucleic acid (“LNA”) sugar, which is a bicyclic sugar. In some embodiments, a polynucleotide for use in the methods described herein comprises one or more nucleotides having an LNA sugar. In some embodiments, a polynucleotide contains one or more regions consisting of nucleotides with LNA sugars. In other embodiments, a polynucleotide contains nucleotides with LNA sugars interspersed with deoxyribonucleotides. See, e.g., Frieden, M. et al. (2008) Curr. Pharm. Des. 14(11):1138-1142.

### 4.1.5.2. Exemplary Primers

In some embodiments, a primer is provided. In some embodiments, a primer is identical or complementary to at least 8, at least 9, at least 10, at least 11, at least 12, at least 13, at least 14, at least 15, at least 16, at least 17, at least 18, at least 19, at least 20, at least 21, at least 22, at least 23, or at least 24 contiguous nucleotides of a target RNA, such as 13629, IL18RAP, 13719, miR-150, 2548, 14689, 14621, or miR-342. In some embodiments, a primer may also comprise portions or regions that are not identical or complementary to the target RNA. In some embodiments, a region of a primer that is identical or complementary to a target RNA is contiguous, such that any region of a primer that is not identical or complementary to the target RNA does not disrupt the identical or complementary region.

In some embodiments, a primer comprises a portion that is identically present in a target RNA, such as at least one RNA selected from 13629, IL18RAP, 13719, miR-150, 2548, 14689, 14621, and miR-342. In some such embodiments, a primer that comprises a region that is identically present in the target RNA is capable of selectively hybridizing to a cDNA that has been reverse transcribed from the RNA, or to an amplicon that has been produced by amplification of the target RNA or cDNA. In some embodiments, the primer is complementary to a sufficient portion of the cDNA or amplicon such that it selectively hybridizes to the cDNA or amplicon under the conditions of the particular assay being used.

As used herein, “selectively hybridize” means that a polynucleotide, such as a primer or probe, will hybridize to a particular nucleic acid in a sample with at least 5-fold greater affinity than it will hybridize to another nucleic acid present in the same sample that has a different nucleotide sequence in the hybridizing region. In some embodiments, a polynucleotide will hybridize to a particular nucleic acid in a sample with at least 10-fold greater affinity than it will hybridize to another nucleic acid present in the same sample that has a different nucleotide sequence in the hybridizing region.

Nonlimiting exemplary primers include primers comprising sequences that are identically present in, or complementary to a region of, at least one RNA selected from 13629, IL18RAP, 13719, miR-150, 2548, 14689, 14621, and miR-342, or another target RNA. Nonlimiting exemplary primers include polynucleotides comprising sequences having at least 8, at least 9, at least 10, at least 11, at least 12, at least 13, at least 14, at least 15, at least 16, at least 17, or at least 18 contiguous nucleotides of a sequence selected from SEQ ID NOs: 37 to 45 and 55; and sequences having at least 8, at least 9, at least 10, at least 11, at least 12, at least 13, at least 14, at least 15, at least 16, at least 17, at least 18, at least 19, at least 20, at least 25, at least 30, at least 40, at least 50, at least 60, or at least 70 contiguous nucleotides of a sequence selected from SEQ ID NOs: 46 to 52, 58, and 59.

In some embodiments, a primer is used to reverse transcribe a target RNA, for example, as discussed herein. In some embodiments, a primer is used to amplify a target RNA or a cDNA reverse transcribed therefrom. Such amplification, in some embodiments, is quantitative PCR, for example, as discussed herein. In some embodiments, a primer comprises a detectable moiety.

### 4.1.5.3. Exemplary Probes

In various embodiments, methods of detecting the presence of a sepsis comprise hybridizing nucleic acids of a sample with a probe. In some embodiments, the probe comprises a portion that is complementary to a target RNA, such as 13629, IL18RAP, 13719, miR-150, 2548, 14689, 14621, and miR-342. In some embodiments, the probe comprises a portion that is identically present in the target RNA, such as 13629, IL18RAP, 13719, miR-150, 2548, 14689, 14621, and miR-342. In some such embodiments, a probe that is complementary to a target RNA is complementary to a sufficient portion of the target RNA such that it selectively hybridizes to the target RNA under the conditions of the particular assay being used. In some embodiments, a probe that is complementary to a target RNA is complementary to at least 8, at least 9, at least 10, at least 11, at least 12, at least 13, at least 14, at least 15, at least 16, at least 17, at least 18, at least 19, at least 20, at least 21, at least 22, at least 23, or at least 24 contiguous nucleotides of the target RNA. In some embodiments, a probe that is complementary to a target RNA comprises a region that is complementary to at least 8, at least 9, at least 10, at least 11, at least 12, at least 13, at least 14, at least 15, at least 16, at least 17, at least 18, at least 19, at least 20, at least 21, at least 22, at least 23, or at least 24 contiguous nucleotides of the target RNA. That is, a probe that is complementary to a target RNA may also comprise portions or regions that are not complementary to the target RNA. In some embodiments, a region of a probe that is complementary to a target RNA is contiguous, such that any region of a probe that is not complementary to the target RNA does not disrupt the complementary region.

In some embodiments, the probe comprises a portion that is identically present in the target RNA, such as 13629, IL18RAP, 13719, miR-150, 2548, 14689, 14621, and miR-342. In some such embodiments, a probe that comprises a region that is identically present in the target RNA is capable of selectively hybridizing to a cDNA that has been reverse transcribed from the RNA, or to an amplicon that has been produced by amplification of the target RNA or cDNA. In some embodiments, the probe is complementary to a sufficient portion of the cDNA or amplicon such that it selectively hybridizes to the cDNA or amplicon under the conditions of the particular assay being used. In some embodiments, a probe that is complementary to a cDNA or amplicon is complementary to at least 8, at least 9, at least 10, at least 11, at least 12, at least 13, at least 14, at least 15, at least 16, at least 17, at least 18, at least 19, at least 20, at least 21, at least 22, at least 23, or at least 24 contiguous nucleotides of the cDNA or amplicon. In some embodiments, a probe that is complementary to a target RNA comprises a region that is complementary to at least 8, at least 9, at least 10, at least 11, at least 12, at least 13, at least 14, at least 15, at least 16, at least 17, at least 18, at least 19, at least 20, at least 21, at least 22, at least 23, or at least 24 contiguous nucleotides of the cDNA or amplicon. That is, a probe that is complementary to a cDNA or amplicon may also comprise portions or regions that are not complementary to the cDNA or amplicon. In some embodiments, a region of a probe that is complementary to a cDNA or amplicon is contiguous, such that any region of a probe that is not complementary to the cDNA or amplicon does not disrupt the complementary region.

Nonlimiting exemplary probes include probes comprising sequences set forth in SEQ ID NOs: 37 to 52, 55, 58, and 59; probes comprising at least 8, at least 9, at least 10, at least 11, at least 12, at least 13, at least 14, at least 15, at least 16, at least 17, or at least 18 contiguous nucleotides of a sequence selected from SEQ ID NOs: 37 to 45 and 55; and probes comprising at least 8, at least 9, at least 10, at least 11, at least 12, at least 13, at least 14, at least 15, at least 16, at least 17, at least 18, at least 19, at least 20, at least 25, at least 30, at least 40, at least 50, at least 60, or at least 70 contiguous nucleotides of a sequence selected from SEQ ID NOs: 46 to 52, 58, and 59.

In some embodiments, the method of detectably quantifying one or more target RNAs comprises: (a) isolating total RNA; (b) reverse transcribing a target RNA to produce a cDNA that is complementary to the target RNA; (c) amplifying the cDNA from (b); and (d) detecting the amount of a target RNA using real time RT-PCR and a detection probe.

As described above, in some embodiments, the real time RT-PCR detection is performed using a FRET probe, which includes, but is not limited to, a TaqMan® probe, a Molecular beacon probe and a Scorpion probe. In some embodiments, the real time RT-PCR detection and quantification is performed with a TaqMan® probe, i.e., a linear probe that typically has a fluorescent dye covalently bound at one end of the DNA and a quencher molecule covalently bound at the other end of the DNA. The FRET probe comprises a sequence that is complementary to a region of the cDNA such that, when the FRET probe is hybridized to the cDNA, the dye fluorescence is quenched, and when the probe is digested during amplification of the cDNA, the dye is released from the probe and produces a fluorescence signal. In such embodiments, the amount of target RNA in the sample is proportional to the amount of fluorescence measured during cDNA amplification.

The TaqMan® probe typically comprises a region of contiguous nucleotides having a sequence that is complementary to a region of a target RNA or its complementary cDNA that is reverse transcribed from the target RNA template (i.e., the sequence of the probe region is complementary to or identically present in the target RNA to be detected) such that the probe is specifically hybridizable to the resulting PCR amplicon. In some embodiments, the probe comprises a region of at least 6 contiguous nucleotides having a sequence that is fully complementary to or identically present in a region of a cDNA that has been reverse transcribed from a target RNA template, such as comprising a region of at least 8 contiguous nucleotides, at least 10 contiguous nucleotides, at least 12 contiguous nucleotides, at least 14 contiguous nucleotides, or at least 16 contiguous nucleotides having a sequence that is complementary to or identically present in a region of a cDNA reverse transcribed from a target RNA to be detected.

In some embodiments, the region of the cDNA that has a sequence that is complementary to the TaqMan® probe sequence is at or near the center of the cDNA molecule. In some embodiments, there are independently at least 2 nucleotides, such as at least 3 nucleotides, such as at least 4 nucleotides, such as at least 5 nucleotides of the cDNA at the 5′-end and at the 3′-end of the region of complementarity.

In some embodiments, Molecular Beacons can be used to detect and quantitate PCR products. Like TaqMan® probes, Molecular Beacons use FRET to detect and quantitate a PCR product via a probe having a fluorescent dye and a quencher attached at the ends of the probe. Unlike TaqMan® probes, Molecular Beacons remain intact during the PCR cycles. Molecular Beacon probes form a stem-loop structure when free in solution, thereby allowing the dye and quencher to be in close enough proximity to cause fluorescence quenching. When the Molecular Beacon hybridizes to a target, the stem-loop structure is abolished so that the dye and the quencher become separated in space and the dye fluoresces. Molecular Beacons are available, e.g., from Gene Link™ (see http://www.genelink.com/newsite/products/mbintro.asp).

In some embodiments, Scorpion probes can be used as both sequence-specific primers and for PCR product detection and quantitation. Like Molecular Beacons, Scorpion probes form a stem-loop structure when not hybridized to a target nucleic acid. However, unlike Molecular Beacons, a Scorpion probe achieves both sequence-specific priming and PCR product detection. A fluorescent dye molecule is attached to the 5′-end of the Scorpion probe, and a quencher is attached to the 3′-end. The 3′ portion of the probe is complementary to the extension product of the PCR primer, and this complementary portion is linked to the 5′-end of the probe by a non-amplifiable moiety. After the Scorpion primer is extended, the target-specific sequence of the probe binds to its complement within the extended amplicon, thus opening up the stem-loop structure and allowing the dye on the 5′-end to fluoresce and generate a signal. Scorpion probes are available from, e.g, Premier Biosoft International (see http://www.premierbiosoft.com/tech_notes/Scorpion.html).

In some embodiments, labels that can be used on the FRET probes include colorimetric and fluorescent labels such as Alexa Fluor dyes, BODIPY dyes, such as BODIPY FL; Cascade Blue; Cascade Yellow; coumarin and its derivatives, such as 7-amino-4-methylcoumarin, aminocoumarin and hydroxycoumarin; cyanine dyes, such as Cy3 and Cy5; eosins and erythrosins; fluorescein and its derivatives, such as fluorescein isothiocyanate; macrocyclic chelates of lanthanide ions, such as Quantum Dye™; Marina Blue; Oregon Green; rhodamine dyes, such as rhodamine red, tetramethylrhodamine and rhodamine 6G; Texas Red; fluorescent energy transfer dyes, such as thiazole orange-ethidium heterodimer; and, TOTAB.

Specific examples of dyes include, but are not limited to, those identified above and the following: Alexa Fluor 350, Alexa Fluor 405, Alexa Fluor 430, Alexa Fluor 488, Alexa Fluor 500. Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, and, Alexa Fluor 750; amine-reactive BODIPY dyes, such as BODIPY 493/503, BODIPY 530/550, BODIPY 558/568, BODIPY 564/570, BODIPY 576/589, BODIPY 581/591, BODIPY 630/650, BODIPY 650/655, BODIPY FL, BODIPY R6G, BODIPY TMR, and, BODIPY-TR; Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, 2′,4′,5′,7′-Tetrabromosulfonefluorescein, and TET.

Specific examples of fluorescently labeled ribonucleotides useful in the preparation of RT-PCR probes for use in some embodiments of the methods described herein are available from Molecular Probes (Invitrogen), and these include, Alexa Fluor 488-5-UTP, Fluorescein-12-UTP, BODIPY FL-14-UTP, BODIPY TMR-14-UTP, Tetramethylrhodamine-6-UTP, Alexa Fluor 546-14-UTP, Texas Red-5-UTP, and BODIPY TR-14-UTP. Other fluorescent ribonucleotides are available from Amersham Biosciences (GE Healthcare), such as Cy3-UTP and Cy5-UTP.

Examples of fluorescently labeled deoxyribonucleotides useful in the preparation of RT-PCR probes for use in the methods described herein include Dinitrophenyl (DNP)-1′-dUTP, Cascade Blue-7-dUTP, Alexa Fluor 488-5-dUTP, Fluorescein-12-dUTP, Oregon Green 488-5-dUTP, BODIPY FL-14-dUTP, Rhodamine Green-5-dUTP, Alexa Fluor 532-5-dUTP, BODIPY TMR-14-dUTP, Tetramethylrhodamine-6-dUTP, Alexa Fluor 546-14-dUTP, Alexa Fluor 568-5-dUTP, Texas Red-12-dUTP, Texas Red-5-dUTP, BODIPY TR-14-dUTP, Alexa Fluor 594-5-dUTP, BODIPY 630/650-14-dUTP, BODIPY 650/665-14-dUTP; Alexa Fluor 488-7-OBEA-dCTP, Alexa Fluor 546-16-OBEA-dCTP, Alexa Fluor 594-7-OBEA-dCTP, Alexa Fluor 647-12-OBEA-dCTP. Fluorescently labeled nucleotides are commercially available and can be purchased from, e.g., Invitrogen.

In some embodiments, dyes and other moieties, such as quenchers, are introduced into polynucleotide used in the methods described herein, such as FRET probes, via modified nucleotides. A “modified nucleotide” refers to a nucleotide that has been chemically modified, but still functions as a nucleotide. In some embodiments, the modified nucleotide has a chemical moiety, such as a dye or quencher, covalently attached, and can be introduced into a polynucleotide, for example, by way of solid phase synthesis of the polynucleotide. In other embodiments, the modified nucleotide includes one or more reactive groups that can react with a dye or quencher before, during, or after incorporation of the modified nucleotide into the nucleic acid. In specific embodiments, the modified nucleotide is an amine-modified nucleotide, i.e., a nucleotide that has been modified to have a reactive amine group. In some embodiments, the modified nucleotide comprises a modified base moiety, such as uridine, adenosine, guanosine, and/or cytosine. In specific embodiments, the amine-modified nucleotide is selected from 5-(3-aminoallyl)-UTP; 8-[(4-amino)butyl]-amino-ATP and 8-[(6-amino)butyl]-amino-ATP; N6-(4-amino)butyl-ATP, N6-(6-amino)butyl-ATP, N4-[2,2-oxy-bis-(ethylamine)]-CTP; N6-(6-Amino)hexyl-ATP; 8-[(6-Amino)hexyl]-amino-ATP; 5-propargylamino-CTP, 5-propargylamino-UTP. In some embodiments, nucleotides with different nucleobase moieties are similarly modified, for example, 5-(3-aminoallyl)-GTP instead of 5-(3-aminoallyl)-UTP. Many amine modified nucleotides are commercially available from, e.g., Applied Biosystems, Sigma, Jena Bioscience and TriLink.

Exemplary detectable moieties also include, but are not limited to, members of binding pairs. In some such embodiments, a first member of a binding pair is linked to a polynucleotide. The second member of the binding pair is linked to a detectable label, such as a fluorescent label. When the polynucleotide linked to the first member of the binding pair is incubated with the second member of the binding pair linked to the detectable label, the first and second members of the binding pair associate and the polynucleotide can be detected. Exemplary binding pairs include, but are not limited to, biotin and streptavidin, antibodies and antigens, etc.

In some embodiments, multiple target RNAs are detected in a single multiplex reaction. In some such embodiments, each probe that is targeted to a unique cDNA is spectrally distinguishable when released from the probe. Thus, each target RNA is detected by a unique fluorescence signal.

One skilled in the art can select a suitable detection method for a selected assay, e.g., a real-time RT-PCR assay. The selected detection method need not be a method described above, and may be any method.

### 4.2. Exemplary Compositions and Kits

In another aspect, compositions are provided. In some embodiments, compositions are provided for use in the methods described herein.

In some embodiments, a composition comprises at least one polynucleotide. In some embodiments, a composition comprises at least one primer. In some embodiments, a composition comprises at least one probe. In some embodiments, a composition comprises at least one primer and at least one probe.

In some embodiments, compositions are provided that comprise at least one target RNA-specific primer. The term “target RNA-specific primer” encompasses primers that have a region of contiguous nucleotides having a sequence that is (i) identically present in a target RNA, such as 13629, IL18RAP, 13719, miR-150, 2548, 14689, 14621, or miR-342, or (ii) complementary to the sequence of a region of contiguous nucleotides found in a target RNA, such as 13629, IL18RAP, 13719, miR-150, 2548, 14689, 14621, or miR-342.

In some embodiments, compositions are provided that comprise at least one target RNA-specific probe. The term “target RNA-specific probe” encompasses probes that have a region of contiguous nucleotides having a sequence that is (i) identically present in a target RNA, such as 13629, IL18RAP, 13719, miR-150, 2548, 14689, 14621, or miR-342, or (ii) complementary to the sequence of a region of contiguous nucleotides found in a target RNA, such as 13629, IL18RAP, 13719, miR-150, 2548, 14689, 14621, or miR-342.

In some embodiments, target RNA-specific primers and probes comprise deoxyribonucleotides. In other embodiments, target RNA-specific primers and probes comprise at least one nucleotide analog. Nonlimiting exemplary nucleotide analogs include, but are not limited to, analogs described herein, including LNA analogs and peptide nucleic acid (PNA) analogs. In some embodiments, target RNA-specific primers and probes comprise at least one nucleotide analog which increases the hybridization binding energy (e.g., an affinity-enhancing nucleotide analog, discussed above). In some embodiments, a target RNA-specific primer or probe in the compositions described herein binds to one target RNA in the sample. In some embodiments, a single primer or probe binds to multiple target RNAs, such as multiple isomirs.

In some embodiments, more than one primer or probe specific for a single target RNA is present in the compositions, the primers or probes capable of binding to overlapping or spatially separated regions of the target RNA.

It will be understood, even if not explicitly stated hereinafter, that in some embodiments in which the compositions described herein are designed to hybridize to cDNAs reverse transcribed from target RNAs, the composition comprises at least one target RNA-specific primer or probe (or region thereof) having a sequence that is identically present in a target RNA (or region thereof).

In some embodiments, a composition comprises a target RNA-specific primer. In some embodiments, the target RNA-specific primer is specific for 13629, IL18RAP, 13719, miR-150, 2548, 14689, 14621, or miR-342. In some embodiments, a composition comprises a plurality of target RNA-specific primers for each of at least 2, at least 3, at least 4, at least 5, at least 6, at least 7, or at least 8 target RNAs.

In some embodiments, a composition comprises a target RNA-specific probe. In some embodiments, the target RNA-specific probe is specific for 13629, IL18RAP, 13719, miR-150, 2548, 14689, 14621, or miR-342. In some embodiments, a composition comprises a plurality of target RNA-specific probes for each of at least 2, at least 3, at least 4, at least 5, at least 6, at least 7, or at least 8 target RNAs.

In some embodiments, a composition is an aqueous composition. In some embodiments, the aqueous composition comprises a buffering component, such as phosphate, tris, HEPES, etc., and/or additional components, as discussed below. In some embodiments, a composition is dry, for example, lyophilized, and suitable for reconstitution by addition of fluid. A dry composition may include a buffering component and/or additional components.

In some embodiments, a composition comprises one or more additional components. Additional components include, but are not limited to, salts, such as NaCl, KCl, and MgCl₂; polymerases, including thermostable polymerases; dNTPs; RNase inhibitors; bovine serum albumin (BSA) and the like; reducing agents, such as β-mercaptoethanol; EDTA and the like; etc. One skilled in the art can select suitable composition components depending on the intended use of the composition.

In some embodiments, a composition comprises RNA of a sample from a subject. The RNA may or may not be separated from one or more other components of the sample. Various RNA separation techniques are known in the art, and are described herein.

In some embodiments, an addressable microarray component is provided that comprises target RNA-specific probes attached to a substrate.

Microarrays for use in the methods described herein comprise a solid substrate onto which the probes are covalently or non-covalently attached. In some embodiments, probes capable of hybridizing to one or more target RNAs or cDNAs are attached to the substrate at a defined location (“addressable array”). Probes can be attached to the substrate in a wide variety of ways, as will be appreciated by those in the art. In some embodiments, the probes are synthesized first and subsequently attached to the substrate. In other embodiments, the probes are synthesized on the substrate. In some embodiments, probes are synthesized on the substrate surface using techniques such as photopolymerization and photolithography.

In some embodiments, the solid substrate is a material that is modified to contain discrete individual sites appropriate for the attachment or association of the probes and is amenable to at least one detection method. Representative examples of substrates include glass and modified or functionalized glass, plastics (including acrylics, polystyrene and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses and plastics. In some embodiments, the substrates allow optical detection without appreciably fluorescing.

In some embodiments, the substrate is planar. In other embodiments, probes are placed on the inside surface of a tube, such as for flow-through sample analysis to minimize sample volume. In other embodiments, probes can be in the wells of multi-well plates. In still other embodiments, probes can be attached to an addressable microbead array. In yet other embodiments, the probes can be attached to a flexible substrate, such as a flexible foam, including closed cell foams made of particular plastics.

The substrate and the probe can each be derivatized with functional groups for subsequent attachment of the two. For example, in some embodiments, the substrate is derivatized with one or more chemical functional groups including, but not limited to, amino groups, carboxyl groups, oxo groups and thiol groups. In some embodiments, probes are attached directly to the substrate through one or more functional groups. In some embodiments, probes are attached to the substrate indirectly through a linker (i.e., a region of contiguous nucleotides that space the probe regions involved in hybridization and detection away from the substrate surface). In some embodiments, probes are attached to the solid support through the 5′ terminus. In other embodiments, probes are attached through the 3′ terminus. In still other embodiments, probes are attached to the substrate through an internal nucleotide. In some embodiments the probe is attached to the solid support non-covalently, e.g., via a biotin-streptavidin interaction, wherein the probe biotinylated and the substrate surface is covalently coated with streptavidin.

In some embodiments, the compositions comprise a microarray having probes attached to a substrate, wherein at least one of the probes (or a region thereof) comprises a sequence that is identically present in, or complementary to a region of, 13629, IL18RAP, 13719, miR-150, 2548, 14689, 14621, or miR-342. In some embodiments, in addition to a probe comprising a sequence that is identically present in, or complementary to a region of, at least one of those RNAs, a microarray further comprises at least one probe comprising a sequence that is identically present in, or complementary to a region of, another target RNA. In some embodiments, in addition to a probe comprising a sequence that is identically present in, or complementary to a region of, at least one of those RNAs, a microarray further comprises at least two, at least five, at least 10, at least 15, at least 20, at least 30, at least 50, or at least 100 probes comprising sequences that are identically present in, or complementary to regions of, other target RNAs. In some embodiments, the microarray comprises each target RNA-specific probe at only one location on the microarray. In some embodiments, the microarray comprises at least one target RNA-specific probe at multiple locations on the microarray.

As used herein, the terms “complementary” or “partially complementary” to a target RNA (or target region thereof), and the percentage of “complementarity” of the probe sequence to that of the target RNA sequence is the percentage “identity” to the reverse complement of the sequence of the target RNA. In determining the degree of “complementarity” between probes used in the compositions described herein (or regions thereof) and a target RNA, such as those disclosed herein, the degree of “complementarity” is expressed as the percentage identity between the sequence of the probe (or region thereof) and the reverse complement of the sequence of the target RNA that best aligns therewith. The percentage is calculated by counting the number of aligned bases that are identical as between the 2 sequences, dividing by the total number of contiguous nucleotides in the probe, and multiplying by 100.

In some embodiments, the microarray comprises at least one probe having a region with a sequence that is fully complementary to a target region of a target RNA. In other embodiments, the microarray comprises at least one probe having a region with a sequence that comprises one or more base mismatches when compared to the sequence of the best-aligned target region of a target RNA.

In some embodiments, the microarray comprises at least one probe having a region of at least 10, at least 11, at least 13, at least 14, at least 15, at least 16, at least 17, at least 18 contiguous nucleotides identically present in, or complementary to, 13629, IL18RAP, 13719, miR-150, 2548, 14689, 14621, or miR-342. In some embodiments, the microarray comprises at least one probe having a region of at least 10, at least 11, at least 13, at least 14, at least 15, at least 16, at least 17, at least 18, at least 19, at least 20, at least 21, at least 22, at least 23, at least 24, or at least 25 contiguous nucleotides with a sequence that is identically present in, or complementary to a region of, another target RNA.

In some embodiments, the microarrays comprise probes having a region with a sequence that is complementary to target RNAs that comprise a substantial portion of the human miRNome (i.e., the publicly known microRNAs that have been accessioned by others into miRBase (http://microrna.sanger.ac.uk/ at the time the microarray is fabricated), such as at least about 60%, at least about 70%, at least about 80%, at least about 90%, even at least about 95% of the human miRNome. In some embodiments, the microarrays comprise probes that have a region with a sequence that is identically present in target RNAs that comprise a substantial portion of the human miRNome, such as at least about 60%, at least about 70%, at least about 80%, at least about 90%, even at least about 95% of the human miRNome.

In some embodiments, components are provided that comprise probes attached to microbeads, such as those sold by Luminex, each of which is internally dyed with red and infrared fluorophores at different intensities to create a unique signal for each bead. In some embodiments, the compositions useful for carrying out the methods described herein include a plurality of microbeads, each with a unique spectral signature. Each uniquely labeled microbead is attached to a unique target RNA-specific probe such that the unique spectral signature from the dyes in the bead is associated with a particular probe sequence. Nonlimiting exemplary probe sequences include SEQ ID NOs: 37 to 52, 55, 58, and 59. Nonlimiting exemplary probe sequences include sequences having at least 8, at least 9, at least 10, at least 11, at least 12, at least 13, at least 14, at least 15, at least 16, at least 17, at least 18 contiguous nucleotides of a sequence selected from SEQ ID NOs: 37 to 45 and 55. Nonlimiting exemplary probe sequences include sequences having at least 8, at least 9, at least 10, at least 11, at least 12, at least 13, at least 14, at least 15, at least 16, at least 17, at least 18, at least 19, at least 20, at least 25, at least 30, at least 40, at least 50, at least 60, or at least 70 contiguous nucleotides of a sequence selected from SEQ ID NOs: 46 to 52, 58, and 59. Nonlimiting exemplary probe sequences also include probes comprising a region that is identically present in, or complementary to, 13629, IL18RAP, 13719, miR-150, 2548, 14689, 14621, or miR-342. Nonlimiting exemplary probe sequences also include probes comprising a region that is identically present in, or complementary to, other target RNAs.

In some embodiments, a uniquely labeled microbead has attached thereto a probe having a region with a sequence that is identically present in, or complementary to a region of, 13629, IL18RAP, 13719, miR-150, 2548, 14689, 14621, or miR-342. In some embodiments, a uniquely labeled microbead has attached thereto a probe comprising a sequence selected from SEQ ID NOs: 37 to 52, 55, 58, and 59. In some embodiments, a uniquely labeled microbead has attached thereto a probe having a region with a sequence having at least 8, at least 9, at least 10, at least 11, at least 12, at least 13, at least 14, at least 15, at least 16, at least 17, at least 18 contiguous nucleotides of a sequence selected from SEQ ID NO: 37 to 45 and 55. In some embodiments, a uniquely labeled microbead has attached thereto a probe having a region with a sequence having at least 8, at least 9, at least 10, at least 11, at least 12, at least 13, at least 14, at least 15, at least 16, at least 17, at least 18, at least 19, at least 20, at least 25, at least 30, at least 40, at least 50, at least 60, or at least 70 contiguous nucleotides of a sequence selected from SEQ ID NOs: 46 to 52, 58, and 59. In some embodiments, a uniquely labeled microbead has attached thereto a probe having a region with a sequence that is identically present in, or complementary to a region of, another target RNA.

In some embodiments, a composition is provided that comprises a plurality of uniquely labeled microbeads, wherein at least one microbead has attached thereto a probe having a region with a sequence that is identically present in, or complementary to a region of, 13629, IL18RAP, 13719, miR-150, 2548, 14689, 14621, or miR-342. In some embodiments, a composition is provided that comprises a plurality of uniquely labeled microbeads, wherein at least one microbead has attached thereto a probe comprising a sequence selected from SEQ ID NOs: 37 to 52, 55, 58, and 59. In some embodiments, a composition is provided that comprises a plurality of uniquely labeled microbeads, wherein at least one microbead has attached thereto a probe having a region with a sequence having at least 8, at least 9, at least 10, at least 11, at least 12, at least 13, at least 14, at least 15, at least 16, at least 17, at least 18 contiguous nucleotides of a sequence selected from SEQ ID NOs: 37 to 45 and 55. In some embodiments, a composition is provided that comprises a plurality of uniquely labeled microbeads, wherein at least one microbead has attached thereto a probe having a region with a sequence having at least 8, at least 9, at least 10, at least 11, at least 12, at least 13, at least 14, at least 15, at least 16, at least 17, at least 18, at least 19, at least 20, at least 25, at least 30, at least 40, at least 50, at least 60, or at least 70 contiguous nucleotides of a sequence selected from SEQ ID NOs: 46 to 52, 58, and 59. In some embodiments, a composition is provided that comprises a plurality of uniquely labeled microbeads, wherein at least one microbead has attached thereto a probe having a region with a sequence that is identically present in, or complementary to a region of, 13629, IL18RAP, 13719, miR-150, 2548, 14689, 14621, or miR-342, and at least one microbead has attached thereto a probe having a region with a sequence that is identically present in, or complementary to a region of, another target RNA.

In some embodiments, the compositions comprise a plurality of uniquely labeled microbeads, each of which has attached thereto a unique probe having a region that is complementary to target RNAs that comprise a substantial portion of the human miRNome, such as at least about 60%, at least about 70%, at least about 80%, at least about 90%, or at least about 95% of the human miRNome. In some embodiments, the compositions comprise a plurality of uniquely labeled microbeads having attached thereto a unique probe having a region with a sequence that is identically present in target RNAs that comprise a substantial portion of the human miRNome, such as at least about 60%, at least about 70%, at least about 80%, at least about 90%, or at least about 95% of the human miRNome.

In some embodiments, compositions are provided that comprise at least one polynucleotide for detecting at least one target RNA. In some embodiments, the polynucleotide is used as a primer for a reverse transcriptase reaction. In some embodiments, the polynucleotide is used as a primer for amplification. In some embodiments, the polynucleotide is used as a primer for RT-PCR. In some embodiments, the polynucleotide is used as a probe for detecting at least one target RNA. In some embodiments, the polynucleotide is detectably labeled. In some embodiments, the polynucleotide is a FRET probe. In some embodiments, the polynucleotide is a TaqMan® probe, a Molecular Beacon, or a Scorpion probe.

In some embodiments, a composition comprises at least one FRET probe having a sequence that is identically present in, or complementary to a region of, 13629, IL18RAP, 13719, miR-150, 2548, 14689, 14621, or miR-342. In some embodiments, a composition comprises at least one FRET probe having a sequence selected from SEQ ID NOs: 37 to 52, 55, 58, and 59. In some embodiments, a composition comprises at least one FRET probe having a region with a sequence having at least 8, at least 9, at least 10, at least 11, at least 12, at least 13, at least 14, at least 15, at least 16, at least 17, at least 18 contiguous nucleotides of a sequence selected from SEQ ID NOs: 37 to 45 and 55. In some embodiments, a composition comprises at least one FRET probe having a region with a sequence having at least 8, at least 9, at least 10, at least 11, at least 12, at least 13, at least 14, at least 15, at least 16, at least 17, at least 18, at least 19, at least 20, at least 25, at least 30, at least 40, at least 50, at least 60, or at least 70 contiguous nucleotides of a sequence selected from SEQ ID NOs: 46 to 52, 58, and 59. In some embodiments, a composition comprises at least one FRET probe having a region with a sequence that is identically present in, or complementary to a region of, 13629, IL18RAP, 13719, miR-150, 2548, 14689, 14621, or miR-342, and at least one FRET probe having a region with a sequence that is identically present in, or complementary to a region of, another target RNA.

In some embodiments, a FRET probe is labeled with a donor/acceptor pair such that when the probe is digested during the PCR reaction, it produces a unique fluorescence emission that is associated with a specific target RNA. In some embodiments, when a composition comprises multiple FRET probes, each probe is labeled with a different donor/acceptor pair such that when the probe is digested during the PCR reaction, each one produces a unique fluorescence emission that is associated with a specific probe sequence and/or target RNA. In some embodiments, the sequence of the FRET probe is complementary to a target region of a target RNA. In other embodiments, the FRET probe has a sequence that comprises one or more base mismatches when compared to the sequence of the best-aligned target region of a target RNA.

In some embodiments, a composition comprises a FRET probe consisting of at least 8, at least 9, at least 10, at least 11, at least 13, at least 14, at least 15, at least 16, at least 17, at least 18, at least 19, at least 20, at least 21, at least 22, at least 23, at least 24, or at least 25 nucleotides, wherein at least a portion of the sequence is identically present in, or complementary to a region of, 13629, IL18RAP, 13719, miR-150, 2548, 14689, 14621, or miR-342. In some embodiments, at least 8, at least 9, at least 10, at least 11, at least 13, at least 14, at least 15, at least 16, at least 17, at least 18, at least 19, at least 20, at least 21, at least 22, at least 23, at least 24, or at least 25 nucleotides of the FRET probe are identically present in, or complementary to a region of, 13629, IL18RAP, 13719, miR-150, 2548, 14689, 14621, or miR-342. In some embodiments, the FRET probe has a sequence with one, two or three base mismatches when compared to the sequence or complement of 13629, IL18RAP, 13719, miR-150, 2548, 14689, 14621, or miR-342.

In some embodiments, the compositions further comprise a FRET probe consisting of at least 10, at least 11, at least 13, at least 14, at least 15, at least 16, at least 17, at least 18, at least 19, at least 20, at least 21, at least 22, at least 23, at least 24, or at least 25 contiguous nucleotides, wherein the FRET probe comprises a sequence that is identically present in, or complementary to a region of, a region of another target RNA. In some embodiments, the FRET probe is identically present in, or complementary to a region of, at least at least 10, at least 11, at least 13, at least 14, at least 15, at least 16, at least 17, at least 18, at least 19, at least 20, at least 21, at least 22, at least 23, or at least 24 contiguous nucleotides of another target RNA.

In some embodiments, a kit comprises a polynucleotide discussed above. In some embodiments, a kit comprises at least one primer and/or probe discussed above. In some embodiments, a kit comprises at least one polymerase, such as a thermostable polymerase. In some embodiments, a kit comprises dNTPs. In some embodiments, kits for use in the real time RT-PCR methods described herein comprise one or more target RNA-specific FRET probes and/or one or more primers for reverse transcription of target RNAs and/or one or more primers for amplification of target RNAs or cDNAs reverse transcribed therefrom.

In some embodiments, one or more of the primers and/or probes is “linear”. A “linear” primer refers to a polynucleotide that is a single stranded molecule, and typically does not comprise a short region of, for example, at least 3, 4 or 5 contiguous nucleotides, which are complementary to another region within the same polynucleotide such that the primer forms an internal duplex. In some embodiments, the primers for use in reverse transcription comprise a region of at least 4, such as at least 5, such as at least 6, such as at least 7 or more contiguous nucleotides at the 3′-end that has a sequence that is complementary to region of at least 4, such as at least 5, such as at least 6, such as at least 7 or more contiguous nucleotides at the 5′-end of a target RNA.

In some embodiments, a kit comprises one or more pairs of linear primers (a “forward primer” and a “reverse primer”) for amplification of a cDNA reverse transcribed from a target RNA, such as 13629, IL18RAP, 13719, miR-150, 2548, 14689, 14621, or miR-342. Accordingly, in some embodiments, a first primer comprises a region of at least 4, at least 5, at least 6, at least 7, at least 8, at least 9, or at least 10 contiguous nucleotides having a sequence that is identical to the sequence of a region of at least 4, at least 5, at least 6, at least 7, at least 8, at least 9, or at least 10 contiguous nucleotides at the 5′-end of a target RNA. Furthermore, in some embodiments, a second primer comprises a region of at least 4, at least 5, at least 6, at least 7, at least 8, at least 9, or at least 10 contiguous nucleotides having a sequence that is complementary to the sequence of a region of at least 4, at least 5, at least 6, at least 7, at least 8, at least 9, or at least 10 contiguous nucleotides at the 3′-end of a target RNA. In some embodiments, the kit comprises at least a first set of primers for amplification of a cDNA that is reverse transcribed from 13629, IL18RAP, 13719, miR-150, 2548, 14689, 14621, or miR-342. In some embodiments, the kit further comprises at least a second set of primers for amplification of a cDNA that is reverse transcribed from another target RNA.

In some embodiments, the kit comprises at least two, at least five, at least 10, at least 15, at least 20, at least 25, at least 30, at least 40, at least 50, at least 60, at least 75, or at least 100 sets of primers, each of which is for amplification of a cDNA that is reverse transcribed from a different target RNA, including 13629, IL18RAP, 13719, miR-150, 2548, 14689, 14621, and/or miR-342. In some embodiments, the kit comprises at least one set of primers that is capable of amplifying more than one cDNA reverse transcribed from a target RNA in a sample.

In some embodiments, probes and/or primers for use in the compositions described herein comprise deoxyribonucleotides. In some embodiments, probes and/or primers for use in the compositions described herein comprise deoxyribonucleotides and one or more nucleotide analogs, such as LNA analogs or other duplex-stabilizing nucleotide analogs described above. In some embodiments, probes and/or primers for use in the compositions described herein comprise all nucleotide analogs. In some embodiments, the probes and/or primers comprise one or more duplex-stabilizing nucleotide analogs, such as LNA analogs, in the region of complementarity.

In some embodiments, the compositions described herein also comprise probes, and in the case of RT-PCR, primers, that are specific to one or more housekeeping genes for use in normalizing the quantities of target RNAs. Such probes (and primers) include those that are specific for one or more products of housekeeping genes selected from U6 snRNA, ACTB, B2M, GAPDH, GUSB, HPRT1, PPIA, RPLP, RRN18S, TBP, TUBB, UBC, YWHA (TATAA), PGK1, and RPL4.

In some embodiments, the kits for use in real time RT-PCR methods described herein further comprise reagents for use in the reverse transcription and amplification reactions. In some embodiments, the kits comprise enzymes such as reverse transcriptase, and a heat stable DNA polymerase, such as Taq polymerase. In some embodiments, the kits further comprise deoxyribonucleotide triphosphates (dNTP) for use in reverse transcription and amplification. In further embodiments, the kits comprise buffers optimized for specific hybridization of the probes and primers.

### 4.2.1. Exemplary Normalization of RNA Levels

In some embodiments, quantitation of target RNA levels requires assumptions to be made about the total RNA per cell and the extent of sample loss during sample preparation. In order to correct for differences between different samples or between samples that are prepared under different conditions, the quantities of target RNAs in some embodiments are normalized to the levels of at least one endogenous housekeeping gene.

Appropriate genes for use as reference genes in the methods described herein include those as to which the quantity of the product does not vary between normal and sepsis samples, or between different cell lines or under different growth and sample preparation conditions. Endogenous housekeeping genes that may be useful as normalization controls in the methods described herein include, but are not limited to, GUSB, U6 snRNA, RNU44, RNU 48, and U47. Further nonlimiting exemplary housekeeping genes that may be useful as normalization controls in the methods described herein include, but are not limited to, ACTB, B2M, GAPDH, HPRT1, PPIA, RPLP, RRN18S, TBP, TUBB, UBC, YWHA (TATAA), PGK1, and RPL4. One skilled in the art will appreciate, however, that many other housekeeping genes not listed here can be used as normalization controls. In some embodiments, one housekeeping gene is used for normalization. In some embodiments, more than one housekeeping gene is used for normalization. In some embodiments, a housekeeping small RNA is used for normalizing a small RNA and a housekeeping mRNA is used for normalizing an mRNA. While the normalization controls are sometimes referred to herein as “housekeeping genes,” one skilled in the art would understand that it is typically not the gene that is being detected, but the transcription product of the gene (e.g., an RNA transcribed from the gene). In some such embodiments, the RNA transcribed from the gene is detected after splicing has occurred.

In some embodiments, small RNA levels are not normalized. In some embodiments, when no suitable referenced gene is available, no normalization is applied. In some such embodiments, the same quantity of total RNA or enriched small RNAs is used for each assay to reduce variability between assays. In some embodiments, one or more mRNAs are normalized to a housekeeping mRNA, and one or more small RNAs are not normalized.

### 4.2.2. Exemplary Qualitative Methods

In some embodiments, methods comprise detecting a qualitative change in a target RNA profile generated from a clinical sample as compared to a normal target RNA profile (in some exemplary embodiments, a target RNA profile of a control sample). Some qualitative changes in the RNA profile are indicative of the presence of sepsis in the subject from which the clinical sample was taken. Various qualitative changes in the RNA profile are indicative of the propensity to proceed to sepsis. The term “target RNA profile” refers to a set of data regarding the concurrent levels of a plurality of target RNAs in the same sample.

In some embodiments, at least one of the target RNAs of the plurality of target RNAs is at least one RNA selected from 13629, IL18RAP, 13719, miR-150, 2548, 14689, 14621, and miR-342. In some embodiments, at least one, at least two, at least three, at least four, or at least five of the target RNAs of the plurality of target RNAs are selected from 13629, IL18RAP, 13719, miR-150, 2548, 14689, 14621, and miR-342. In some embodiments, the plurality of target RNAs comprises at least one, at least two, at least five, at least 10, at least 15, at least 20, at least 25, at least 30, at least 40, at least 50, at least 60, at least 75, or at least 100 additional target RNAs. In some embodiments, a target RNA, in its mature form, comprises fewer than 30 nucleotides. In some embodiments, a target RNA is a microRNA. In some embodiments, a target RNA is a small cellular RNA.

Qualitative data for use in preparing target RNA profiles is obtained using any suitable analytical method, including the analytical methods presented herein.

In some embodiments, for example, concurrent RNA profile data are obtained using, e.g., a microarray, as described above. Thus, in addition to use for quantitatively determining the levels of specific target RNAs as described above, a microarray comprising probes having sequences that are complementary to a substantial portion of the miRNome may be employed to carry out target RNA profiling, for analysis of target RNA expression patterns.

According to the RNA profiling method, in some embodiments, total RNA from a sample from a subject suspected of having sepsis is quantitatively reverse transcribed to provide a set of labeled polynucleotides complementary to the RNA in the sample. The polynucleotides are then hybridized to a microarray comprising target RNA-specific probes to provide a hybridization profile for the sample. The result is a hybridization profile for the sample representing the target RNA profile of the sample. The hybridization profile comprises the signal from the binding of the polynucleotides reverse transcribed from the sample to the target RNA-specific probes in the microarray. In some embodiments, the profile is recorded as the presence or absence of binding (signal vs. zero signal). In some embodiments, the profile recorded includes the intensity of the signal from each hybridization. The profile is compared to the hybridization profile generated from a healthy individual, or in some embodiments, a control sample. An alteration in the signal is indicative of the presence of sepsis in the subject.

### 4.3. Exemplary Additional Target RNAs and Additional Markers

In some embodiments, in combination with detecting at least one RNA selected from 13629, IL18RAP, 13719, miR-150, 2548, 14689, 14621, and miR-342, a method comprises detecting one or more additional target RNAs. Additional target RNAs include, but are not limited to, microRNAs, other small cellular RNAs, and mRNAs. In some embodiments, one or more additional target RNAs that have been shown to correlate with sepsis in general, or a particular type or stage of sepsis, are selected.

In some embodiments, the methods described herein further comprise detecting chromosomal codependents, i.e., target RNAs clustered near each other in the human genome which tend to be regulated together. Accordingly, in further embodiments, the methods comprise detecting the expression of one or more target RNAs, each situated within the chromosome no more than 50,000 bp from the chromosomal location of an RNA selected from 13629, IL18RAP, 13719, miR-150, 2548, 14689, 14621, and miR-342.

Any of the methods described herein may further comprise detection of one or more additional markers of sepsis and/or one or more additional markers that aid in distinguishing sepsis from one or more other conditions, such as SIRS. Nonlimiting exemplary additional markers include procalcitonin (PCT), CD64, C-reactive protein (CRP), IL-18, serum lactate, IL-2, and IL-8. Procalcitonin is a peptide precursor of the hormone calcitonin, which is involved in calcium homeostasis. Elevated blood procalcitonin levels have been found, in some instances, to be good indicators of sepsis, and to aid in differentiation of sepsis and SIRS. See, e.g., Meisner et al., Crit Care 1999, 3: 45-50; Balci et al., Crit Care 2003, 7: 85-90. In some embodiments, PCT levels are compared to levels of one or more of C-reactive protein, IL-2, IL-6, IL-8, and/or TNF-α. Elevated levels of CRP, IL-2, IL-18, IL-8, and serum lactate have also been found to correlate with sepsis severity. See, e.g., Balci et al., Crit Care 2003, 7: 85-90; Castelli et al., Crit Care, 8: R234-R242; Povoa, Intensive Care Med 2002, 28: 235-243; Tschoeke et al., Crit Care Med., 2006, 34: 1225-1233; Mikkelsen et al., Crit Care Med., 2009, 37: 1670-1677. Expression of CD64 by neutrophilic granulocytes has also been shown to be a marker for sepsis. See, e.g., Hoffmann, Clin Chem Lab Med, 2009, 47: 903-916; U.S. Pat. No. 8,116,984.

In some embodiments, one or more additional markers are detected at the protein level. In some embodiments, one or more additional markers are detected on a cell surface, such as by fluorescence activated cell sorting (FACS). In some embodiments, one or more additional markers are detected at the mRNA level. In some embodiments, one or more additional markers are detected in a separate assay from the RNAs described herein. In some embodiments, one or more additional markers are detected in the same assay as at least one RNA described herein.

### 4.4. Pharmaceutical Compositions and Methods of Treatment

In some embodiments, a method is provided that comprises detecting the presence of sepsis in a subject, and if sepsis is present, treating the subject for sepsis. In some embodiments, the method comprises detecting the level of at least one RNA selected from 2548, IL18RAP, 14689, 14621, miR-342, 13629, 13719, and miR-150, wherein detection of a level of 2548, 14689, miR-342, or miR-150 that is less than a normal level of the respective RNA, and wherein detection of a level of IL18RAP, 14621, 13629, or 13719 that is greater than a normal level of the respective RNA, indicates the presence of sepsis in the subject; and if sepsis is present, treating the subject for sepsis. In some embodiments, the method comprises detecting the levels of at least two, at least three, at least four, at least five, or at least six RNAs selected from 13629, IL18RAP, 13719, miR-150, 2548, 14689, 14621, and miR-342. In some embodiments, the method comprises detecting the levels of 13629-L, 13629-R, and miR-150. In some such embodiments, the method comprises detecting one or more additional RNAs.

In some embodiments, a method comprises detecting in a subject a level of at least one RNA selected from 2548, 14689, miR-342, and miR-150 that is less than a normal level of the respective RNA and/or detecting a level of at least one RNA selected from IL18RAP, 14621, 13629, and 13719 that is greater than a normal level of the respective RNA; and treating the subject for sepsis. In some embodiments, the method comprises detecting the levels of 13629-L, 13629-R, and miR-150. In some such embodiments, the method comprises detecting one or more additional RNAs.

In some embodiments, treating the subject for sepsis comprises at least one treatment selected from administering one or more antibiotics, administering a vasopressor, administering fluids, and administering oxygen. Nonlimiting exemplary antibiotics that may be administered include broad-spectrum antibiotics, such as amoxicillin, imipenem, levofloxacin, gatifloxacin, moxifloxacin, and ampicillin; and narrow-spectrum antibiotics, such as azithromycin, clarithromycin, clindamycin, erythromycin, and vancomycin. In some embodiments, an antibiotic is administered intravenously. Nonlimiting exemplary vasopressors that may be administered include norepinephrine and dopamine. In some embodiments, a vasopressor is administered intravenously. Nonlimiting exemplary fluids that may be administered include crystalloid fluids, such as saline; and colloid fluids, such as albumin solutions and dextran solutions. In some embodiments, a fluid is administered intravenously. Oxygen, in some embodiments, is administered using a nasal cannula or a face mask (such as a simple face mask, air-entrainment mask, partial rebreathing mask, non-rebreather mask, bag-valve-mask, an oxygen resuscitator, etc.).

In some embodiments, a subject is treated for sepsis less than 8 hours, less than 7 hours, less than 6 hours, less than 5 hours, less than 4 hours, or less than 3 hours after a sample is obtained from the subject. In some embodiments, a method of detecting the presence of sepsis described herein is carried out in the time between sample collection and treatment for sepsis. That is, in some embodiments, a sample is collected, a method of detecting sepsis, e.g., by detecting the level of at least one RNA, is carried out, and depending on the result of the detection assay, the subject is or is not treated for sepsis, all within the time frame indicated (i.e., within less than 8 hours, less than 7 hours, less than 6 hours, less than 5 hours, less than 4 hours, or less than 3 hours after a sample is obtained from the subject).

In some embodiments, the disclosure relates to methods of treating sepsis in which expression of a target RNA is deregulated, e.g., either down-regulated or up-regulated. In some embodiments, the disclosure relates to methods of treating sepsis in which levels of a target RNA are altered relative to normal cells, whole blood, and/or serum, e.g., either lower or higher. When at least one isolated target RNA is up-regulated in sepsis, such as 13629, IL18RAP, or 13719, the method comprises administering to the individual an effective amount of at least one compound that inhibits the expression of the at least one target RNA. Alternatively, in some embodiments, when at least one target RNA is up-regulated in the sepsis sample, the method comprises administering to the individual an effective amount of at least one compound that inhibits the activity of the at least one target RNA. Such a compound may be, in some embodiments, a polynucleotide, including a polynucleotide comprising modified nucleotides.

When at least one target RNA is down-regulated in sepsis, such as 2548, 14689, miR-150, or miR-342, the method comprises administering an effective amount of an isolated target RNA (i.e., in some embodiments, a target RNA that is chemically synthesized, recombinantly expressed or purified from its natural environment), or an isolated variant or biologically-active fragment thereof.

The disclosure further provides pharmaceutical compositions for treating sepsis. In some embodiments, the pharmaceutical composition comprises a compound that inhibits the expression of, or the activity of, 13629, IL18RAP, and/or 13719. In some embodiments, the pharmaceutical compositions comprise at least one isolated target RNA, or an isolated variant or biologically-active fragment thereof, and a pharmaceutically-acceptable carrier. In some embodiments, the at least one isolated target RNA corresponds to a target RNA, such as 2548, 14689, miR-150, or miR-342, that is present at decreased levels in sepsis relative to normal levels (in some exemplary embodiments, relative to the level of the target RNA in a control sample).

In some embodiments the isolated target RNA is identical to an endogenous wild-type target RNA gene product that is down-regulated in sepsis. In some embodiments, the isolated target RNA is a variant target RNA or biologically active fragment thereof. As used herein, a “variant” refers to a target RNA gene product that has less than 100% sequence identity to the corresponding wild-type target RNA, but still possesses one or more biological activities of the wild-type target RNA (e.g., ability to inhibit expression of a target RNA molecule and cellular processes associated with sepsis). A “biologically active fragment” of a target RNA is a fragment of the target RNA gene product that possesses one or more biological activities of the wild-type target RNA. In some embodiments, the isolated target RNA can be administered with one or more additional anti-sepsis treatments, such as antibiotic therapy. In some embodiments, the isolated target RNA is administered concurrently with additional anti-sepsis treatments. In some embodiments, the isolated target RNA is administered sequentially to additional anti-sepsis treatments.

In some embodiments, the pharmaceutical compositions comprise at least one compound that inhibits the expression or activity of a target RNA. In some embodiments, the compound is specific for one or more target RNAs, the levels of which are increased in sepsis relative to normal levels (in some exemplary embodiments, relative to the level of the target RNA in a control sample). In some embodiments, the target RNA inhibitor is specific for a particular target RNA, such as 13629, IL18RAP, or 13719. In some embodiments, the target RNA inhibitor comprises a nucleotide sequence that is complementary to at least a portion of 13629, IL18RAP, 13719, and/or other target RNA.

In some embodiments, the target RNA inhibitor is selected from double-stranded RNA, antisense nucleic acids and enzymatic RNA molecules. In some embodiments, the target RNA inhibitor is a small molecule inhibitor. In some embodiments, the target RNA inhibitor can be administered in combination with other anti-sepsis treatments, such as antibiotic therapy. In some embodiments, the target RNA inhibitor is administered concurrently with other anti-sepsis treatments. In some embodiments, the target RNA inhibitor is administered sequentially to other anti-sepsis treatments.

In some embodiments, a pharmaceutical composition is formulated and administered according to Semple et al., Nature Biotechnology advance online publication, 17 Jan. 2010 (doi:10.1038/nbt.1602)), which is incorporated by reference herein in its entirety for any purpose.

The terms “treat,” “treating” and “treatment” as used herein refer to ameliorating symptoms associated with sepsis, including preventing or delaying the onset of symptoms and/or lessening the severity or frequency of symptoms of sepsis.

The term “effective amount” of a target RNA or an inhibitor of target RNA expression or activity is an amount sufficient to prevent or reverse the development of sepsis. An effective amount of a compound for use in the pharmaceutical compositions disclosed herein is readily determined by a person skilled in the art, e.g., by taking into account factors such as the size and weight of the individual to be treated, the stage of the disease, the age, health and gender of the individual, the route of administration, etc.

In addition to an isolated target RNA or a target RNA inhibitor, or a pharmaceutically acceptable salt thereof, the pharmaceutical compositions disclosed herein further comprise a pharmaceutically acceptable carrier, including but not limited to, water, buffered water, normal saline, 0.4% saline, 0.3% glycine, and hyaluronic acid. In some embodiments, the pharmaceutical compositions comprise an isolated target RNA or a target RNA inhibitor that is encapsulated, e.g., in liposomes. In some embodiments, the pharmaceutical compositions comprise an isolated target RNA or a target RNA inhibitor that is resistant to nucleases, e.g., by modification of the nucleic acid backbone as described above in Section 4.1.5. In some embodiments, the pharmaceutical compositions further comprise pharmaceutically acceptable excipients such as stabilizers, antioxidants, osmolality adjusting agents and buffers. In some embodiments, the pharmaceutical compositions further comprise at least one chemotherapeutic agent, including but not limited to, alkylating agents, anti-metabolites, epipodophyllotoxins, anthracyclines, vinca alkaloids, plant alkaloids and terpenoids, monoclonal antibodies, taxanes, topoisomerase inhibitors, platinum compounds, protein kinase inhibitors, and antisense nucleic acids.

Pharmaceutical compositions can take the form of solutions, suspensions, emulsions, tablets, pills, pellets, capsules, capsules containing liquids, powders, sustained-release formulations, suppositories, emulsions, aerosols, sprays, suspensions, or any other form suitable for use. Methods of administration include, but are not limited to, oral, parenteral, intravenous, oral, and by inhalation.

The following examples are for illustration purposes only, and are not meant to be limiting in any way.

### 5. EXAMPLES

### 5.1 Example 1

### RNA Levels Determined by Quantitative RT-PCR in Sepsis and SIRS Patient Samples

### Patients

The clinical study was carried out in the Intensive Care Unit (ICU) at Guy's and St. Thomas' Hospital, London. The study was approved by and conducted under the rules and regulations of the Ethics Committee and R&D Department of Guy's and St. Thomas' Hospital NHS Trust (REC reference No. 08/H0802/110). Eligible patients and healthy volunteers were given patient information sheets and were consented by research nurses. For each individual, 5 ml of blood was taken by venepuncture and blood from ICU patients was obtained from existing central venous catheters using EDTA anti-coagulated Vacutainers (BD Biosciences, NJ, USA). Whole blood was stored at 4° C. before transfer to the research lab for processing. All studies were carried out in a double-blind fashion with research nurses responsible for taking samples and collecting clinical information, and scientists generating laboratory results without knowing the nature of samples.

Patients were identified by 5 research nurses who were located in ICU and skilled in routine screening of patients for entry into clinical trials by assessing the electronic ‘Careview’ patient information system at least daily. Patients with a systemic inflammatory response syndrome caused by bacterial infection (i.e., sepsis) or caused by non-infectious sources (i.e., SIRS) were identified and consented by a nurse. The inclusive and exclusive criteria for the study were:
Sepsis: two or more SIRS manifestations selected from the Modified SIRS Criteria (temperature>38° C. or ≦36° C.; heart beat>90 beats/minute (except if known cause); respiratory rate<20 breaths/minute or PaCO₂≦32 mmHg or use of mechanical ventilation; and white blood cell count>12,000/mm³ or ≦4,000/mm3 or <10% immature neutrophils) with a clinically suspected source of infection at the time of recruitment;
SIRS: two or more SIRS manifestations selected from the Modified SIRS Criteria (temperature>38° C. or ≦36° C.; heart beat>90 beats/minute (except if known cause); respiratory rate<20 breaths/minute or PaCO₂≦32 mmHg or use of mechanical ventilation; and white blood cell count>12,000/mm³ or ≦4,000/mm3 or <10% immature neutrophils) with no suspected source of infection at the time of recruitment.
   The determination of the presence or absence of an infection was made by the nurse in discussions with the attending physician. In addition, patients were excluded if they were less than 18 years old, pregnant, and/or more than 48 hours had passed since the first sign of inflammation. Healthy subjects were selected to age and gender match the patient group from emergency rooms. 25 patients were recruited for each group.

Within the sepsis group, seven patients tested positive for Gram negative bacterial infection (two of the patients had bacteremia), three patients tested positive for Gram positive bacterial infection, and one patient tested positive for both Gram negative and Gram positive bacterial infections. The remaining sepsis patients were diagnosed clinically. The following table shows a summary of certain demographic and clinical information for the patients in the study.

| | Mean | | SOFA² | | |
|---|---|---|---|---|---|
| | Age | APACHEII¹ | Score | | |
| Group* | (SD) | Score (SD) | (SD) | Diagnosis | Microbiological report |
| 1 | 43 (9) | n/a | n/a | Healthy individual | N/A |
| 2 | 57 (17) | 12.5 (5.0) | 3.3 | SIRS patients: | No known infection |
| | | | (2.4) | includes Coronary Artery | |
| | | | | Bypass Grafts | |
| | | | | (n = 9); Aortic dissection (n = 2); | |
| | | | | Aortic Valve Replacement | |
| | | | | (n = 1); Trans Apical Aortic | |
| | | | | Valve Replacement (n = 1); Burn | |
| | | | | (n = 1); Abdominal Aortic | |
| | | | | Aneurysm (n = 1); Post-operative | |
| | | | | trauma (n = 4); and Others | |
| 3 | 62 (14) | 18.6 (6.5) | 6.0 | Sepsis Patients: | G−ve. Sepsis (n = 7, 2 |
| | | | (4.2) | includes Pneumonia (n = 4); | bacteremia) |
| | | | | Chest Infection (n = 3); | G+ve. Sepsis (n = 3) |
| | | | | Septicaemia (n = 3); Intra- | 1 case with G+ve. & G−ve. |
| | | | | abdominal Sepsis (n = 3); Acute | bacteremia |
| | | | | Kidney Injury (n = 2); Biliary | |
| | | | | Sepsis (n = 2); Peritonitis (n = 2); | |
| | | | | and Others | |

*Group: 1) Healthy volunteers; 2) SIRS patients; 3) Sepsis patients
¹Acute Physiology and Chronic Health Evaluation
²Sequential Organ Failure Assessment

Patients with sepsis had significantly higher white blood cell counts (p=0.027) and C-reactive protein (p<0.001) compared to SIRS patients, but no difference in temperature. Nonlimiting exemplary infectious species identified in the sepsis patients include Pseudomonas aeruginosa, Klebsiella species, Enterococcus faecalis, Haemophilus species, Staphylococcus aureus, E. coli, Citrobacter koseri, and Corynebacterium species.

### RNA Preparation

RNA extraction was performed using a standard TRIzol LS protocol (Sigma-Aldrich, USA). Whole blood was stored in 3 volumes of TRIzol LS for homogenizing. 200 μl chloroform was added to each sample containing 750 μl TRIzol, the samples were mixed and incubated for 5 minutes at room temperature. After incubation, the samples were centrifuged at 12,000×g for 15 minutes at 4° C. and the aqueous phase was transferred to a fresh tube. 1.5 volumes of absolute ethanol were added to the aqueous phase. 700 μl of the ethanol mixture was added to an RNAeasy mini spin column (Qiagen, USA). The columns were centrifuged at 8,000×g for 15 seconds. The columns were washed with RWT buffer and RPE buffer (Qiagen, USA) according to the manufacturer's instructions. RNA was then eluted using 50 μl RNAse-free water. RNA quality was evaluated using a NanoDrop spectrophotometer (ThermoScientific, USA).

### Quantitative RT-PCR Reactions

Small RNA levels were detected by qRT-PCR using Exiqon custom LNA primers (Exiqon, Vedbaek, DK), according to the manufacturer's instructions. Experiments were performed in triplicate wells. No normalization was applied to 13629-L, 13629-R, 13719-L, miR-150, 2548-L, 14689-L, 14621-L, and miR-342-3p target genes.

IL18RAP levels were detected using an ABI Taqman® assay with primers spanning exons 5 and 6. IL18RAP levels were normalized to GUSB, which was also detected using an ABI Taqman® assay.

### Statistical Analysis of RT-PCR Data

Microsoft Excel 2007, SPSS 17.0, and Graphpad Prism 4.0 were used to construct graphs and for statistical analysis. The Shapiro-Wilk normality test was used to compare the distribution of data from measured small RNAs to those of the Gaussian distribution. Group differences were tested using analysis of variance (ANOVA). Pairwise, group comparisons after ANOVA were carried out using Tukey's HSD multiple comparison test.

Differences were considered statistically significant at probability (p) values of less than 0.05. R software was employed for statistical analyses. See, e.g., R Development Core Team (2010). R: A language and environment for statistical computing. R Foundation for Statistical Computing, Vienna, Austria. ISBN 3-900051-07-0, URL http://www.R-project.org/.

### Results

Exemplary average Cts from three reactions is shown in Table 1 for each small RNA. Some patient samples produced no results for one or more of the microRNAs (e.g., the reaction failed). Accordingly, the results shown in each row of Table 1 do not necessarily reflect the results from a single patient. 14621-L levels were also detected in one additional SIRS sample not shown in Table 1. The Ct for that additional SIRS sample was 21.93.

TABLE 1
Average Ct values for 13629-L, 13629-R, 13719-L, miR-150,
2548-L, 14689-L, 14621-L, and miR-342-3p

| | Ct | Ct | Ct | Ct | | Ct | Ct | Ct |
|---|---|---|---|---|---|---|---|---|
| Patient | 13629-L | 13629-R | 13719-L | miR-150 | Ct 2548 | 14689 | 14621-L | miR-342-3p |
| Healthy 1 | 33.02 | 31.79 | 33.65 | 20.51 | 18.58 | 29.25 | 21.10 | 22.59 |
| Healthy 2 | 32.85 | 32.01 | 37.80 | 21.25 | 18.60 | 29.75 | 21.47 | 22.65 |
| Healthy 3 | 32.89 | 32.31 | 33.71 | 20.40 | 17.82 | 28.52 | 21.34 | 22.72 |
| Healthy 4 | 31.98 | 31.96 | 32.28 | 21.28 | 17.76 | 29.52 | 21.32 | 23.05 |
| Healthy 5 | 31.49 | 30.49 | 37.40 | 19.43 | 19.12 | 29.41 | 21.06 | 22.58 |
| Healthy 6 | 31.56 | 29.90 | 33.44 | 18.35 | 17.36 | 28.29 | 19.99 | 20.88 |
| Healthy 7 | 33.85 | 31.10 | 33.42 | 18.95 | 17.55 | 29.81 | 21.58 | 21.01 |
| Healthy 8 | 32.74 | 30.61 | 33.13 | 19.49 | 18.36 | 29.88 | 22.58 | 23.11 |
| Healthy 9 | 31.87 | 31.20 | 33.87 | 19.33 | 18.81 | 29.90 | 21.24 | 23.10 |
| Healthy 10 | 32.49 | 32.53 | 35.88 | 19.86 | 17.92 | 29.46 | 21.00 | 22.58 |
| Healthy 11 | 32.26 | 31.42 | 32.85 | 19.77 | 17.36 | 28.91 | 20.61 | 21.89 |
| Healthy 12 | 31.24 | 30.14 | 33.01 | 19.37 | 16.57 | 27.16 | 19.76 | 21.91 |
| Healthy 13 | 31.45 | 31.59 | 33.22 | 19.27 | 16.53 | 27.85 | 20.68 | 21.01 |
| Healthy 14 | 30.93 | 31.08 | 33.80 | 19.41 | 18.08 | 29.15 | 21.84 | 23.22 |
| Healthy 15 | 31.54 | 30.17 | 35.81 | 19.63 | 17.87 | 29.61 | 20.74 | 22.56 |
| Healthy 16 | 32.21 | 31.24 | 33.64 | 20.21 | 17.60 | 29.68 | 21.41 | 22.33 |
| Sepsis 1 | 32.18 | 31.59 | 31.42 | 22.13 | 19.42 | 30.58 | 20.51 | 24.63 |
| Sepsis 2 | 30.91 | 28.79 | 30.72 | 23.31 | 19.78 | 29.80 | 19.64 | 24.65 |
| Sepsis 3 | 31.21 | 29.99 | 31.05 | 22.41 | 17.87 | 29.61 | 19.25 | 23.45 |
| Sepsis 4 | 28.87 | 27.29 | 30.70 | 23.12 | 18.40 | 30.40 | 17.79 | 23.91 |
| Sepsis 5 | 30.78 | 29.23 | 31.07 | 23.44 | 19.26 | 30.70 | 20.58 | 24.97 |
| Sepsis 6 | 29.63 | 27.72 | 32.80 | 17.69 | 17.22 | 29.98 | 20.80 | 22.19 |
| Sepsis 7 | 30.28 | 30.21 | 31.28 | 23.29 | 19.93 | 31.30 | 17.78 | 25.05 |
| Sepsis 8 | 31.25 | 30.25 | 32.35 | 24.79 | 20.26 | 31.61 | 20.19 | 25.82 |
| Sepsis 9 | 29.84 | 28.21 | 32.11 | 23.17 | 18.08 | 30.09 | 19.62 | 23.72 |
| Sepsis 10 | 26.60 | 25.61 | 29.87 | 20.16 | 17.86 | 29.08 | 18.19 | 23.49 |
| Sepsis 11 | 30.35 | 31.03 | 31.75 | 21.24 | 18.35 | 29.31 | 21.35 | 23.15 |
| Sepsis 12 | 29.92 | 28.24 | 32.64 | 23.27 | 17.96 | 30.05 | 19.83 | 23.71 |
| Sepsis 13 | 28.91 | 28.39 | 30.77 | 22.31 | 18.79 | 30.80 | 17.83 | 23.49 |
| Sepsis 14 | 30.96 | 30.31 | 31.92 | 22.40 | 18.68 | 30.07 | 19.40 | 23.68 |
| Sepsis 15 | 30.32 | 29.62 | 31.84 | 20.88 | 17.98 | 29.79 | 20.43 | 23.30 |
| Sepsis 16 | 30.98 | 30.35 | 30.20 | 22.58 | 19.82 | 30.94 | 18.51 | 24.70 |
| Sepsis 17 | 31.33 | 30.41 | 32.80 | 22.46 | 20.61 | 31.77 | 21.16 | 26.04 |
| Sepsis 18 | 29.21 | 28.36 | 32.75 | 21.84 | 18.44 | 29.69 | 19.73 | 23.69 |
| Sepsis 19 | 30.00 | 29.27 | 30.74 | 23.33 | 18.60 | 29.99 | 19.65 | 24.16 |
| Sepsis 20 | 30.19 | 29.94 | 31.41 | 22.29 | 19.79 | 30.74 | 18.63 | 23.87 |
| Sepsis 21 | 29.77 | 28.65 | 31.36 | 23.28 | 17.80 | 29.64 | 18.57 | 23.62 |
| Sepsis 22 | 32.47 | 31.75 | 32.64 | 22.54 | 19.39 | 30.93 | 21.28 | 23.76 |
| SIRS 1 | 31.67 | 30.66 | 34.72 | 20.24 | 17.48 | 29.22 | 20.46 | 21.81 |
| SIRS 2 | 31.12 | 29.58 | 32.21 | 20.45 | 17.46 | 29.30 | 19.68 | 22.25 |
| SIRS 3 | 31.14 | 29.31 | 32.75 | 22.46 | 18.91 | 30.56 | 20.45 | 23.71 |
| SIRS 4 | 31.38 | 29.66 | 31.63 | 19.49 | 17.48 | 29.35 | 19.84 | 22.01 |
| SIRS 5 | 31.14 | 29.67 | 32.79 | 21.38 | 19.02 | 29.84 | 20.87 | 24.22 |
| SIRS 6 | 30.75 | 29.08 | 31.80 | 22.24 | 16.74 | 28.62 | 20.09 | 22.09 |
| SIRS 7 | 31.60 | 30.05 | 31.19 | 20.38 | 18.35 | 30.17 | 20.14 | 22.76 |
| SIRS 8 | 31.12 | 30.10 | 32.57 | 20.49 | 18.04 | 29.00 | 20.09 | 22.53 |
| SIRS 9 | 32.30 | 31.07 | 32.64 | 19.59 | 18.02 | 28.65 | 20.00 | 21.95 |
| SIRS 10 | 31.10 | 29.52 | 31.28 | 19.75 | 17.93 | 29.94 | 19.73 | 22.31 |
| SIRS 11 | 32.58 | 31.76 | 32.50 | 22.31 | 19.15 | 29.38 | 21.71 | 24.26 |
| SIRS 12 | 32.58 | 30.92 | 31.44 | 21.42 | 18.32 | 29.54 | 21.58 | 23.20 |
| SIRS 13 | 32.87 | 31.45 | 32.79 | 22.07 | 19.08 | 30.03 | 21.09 | 23.79 |
| SIRS 14 | 33.56 | 33.21 | 33.14 | 22.52 | 18.99 | 30.43 | 20.87 | 23.75 |
| SIRS 15 | 31.26 | 30.74 | 32.04 | 21.20 | 17.75 | 29.17 | 20.72 | 23.31 |
| SIRS 16 | 31.85 | 30.57 | 31.40 | 22.34 | 18.48 | 30.45 | 21.17 | 23.82 |
| SIRS 17 | 30.78 | 29.73 | 31.04 | 21.16 | 18.15 | 29.69 | 19.75 | 22.95 |
| SIRS 18 | 30.83 | 29.21 | 32.79 | 21.16 | 17.79 | 29.53 | 21.08 | 23.12 |
| SIRS 19 | 30.39 | 29.08 | 31.32 | 20.50 | 17.91 | 28.88 | 19.97 | 22.59 |
| SIRS 20 | 31.21 | 30.66 | 31.82 | 23.19 | 18.97 | 30.44 | 21.87 | 23.84 |
| SIRS 21 | 31.62 | 31.11 | 31.46 | 22.17 | 17.92 | 28.70 | 21.59 | 22.99 |
| SIRS 22 | 30.67 | 30.17 | 30.27 | 22.80 | 18.82 | 30.17 | 19.16 | 24.42 |

The average Ct from three reactions is shown in Table 2 for GUSB and IL18RAP, as well as the calculation IL18RAP-GUSB.

TABLE 2
Average Ct values for GUSB, IL18RAP, and IL18RAP − GUSB

| | | | IL18RAP − |
|---|---|---|---|
| Patient | GUSB | IL18RAP | GUSB |
| Healthy 1 | 33.85 | 33.40 | −0.45 |
| Healthy 2 | 33.12 | 33.53 | 0.40 |
| Healthy 3 | 33.24 | 32.51 | −0.72 |
| Healthy 4 | 31.41 | 31.74 | 0.33 |
| Healthy 5 | 31.83 | 30.89 | −0.93 |
| Healthy 6 | 32.82 | 33.67 | 0.84 |
| Healthy 7 | 31.75 | 32.88 | 1.13 |
| Healthy 8 | 31.96 | 31.38 | −0.57 |
| Healthy 9 | 31.77 | 32.23 | 0.46 |
| Healthy 10 | 32.71 | 31.59 | −1.12 |
| Healthy 11 | 32.33 | 32.29 | −0.04 |
| Healthy 12 | 33.12 | 32.26 | −0.86 |
| Healthy 13 | 31.04 | 31.78 | 0.75 |
| Healthy 14 | 32.79 | 32.50 | −0.29 |
| Sepsis 1 | 40.00 | 38.29 | −1.71 |
| Sepsis 2 | 34.08 | 32.56 | −1.52 |
| Sepsis 3 | 33.14 | 30.91 | −2.22 |
| Sepsis 4 | 31.99 | 28.95 | −3.04 |
| Sepsis 5 | 31.85 | 28.12 | −3.72 |
| Sepsis 6 | 32.22 | 31.28 | −0.93 |
| Sepsis 7 | 32.66 | 32.95 | 0.29 |
| Sepsis 8 | 33.62 | 31.09 | −2.53 |
| Sepsis 9 | 35.11 | 31.45 | −3.66 |
| Sepsis 10 | 32.95 | 31.44 | −1.50 |
| Sepsis 11 | 33.36 | 30.16 | −3.20 |
| Sepsis 12 | 32.00 | 28.25 | −3.76 |
| Sepsis 13 | 33.13 | 31.72 | −1.40 |
| Sepsis 14 | 31.24 | 30.44 | −0.80 |
| Sepsis 15 | 32.23 | 30.82 | −1.41 |
| Sepsis 16 | 31.74 | 31.85 | 0.12 |
| Sepsis 17 | 31.46 | 29.14 | −2.32 |
| Sepsis 18 | 34.38 | 31.62 | −2.76 |
| Sepsis 19 | 32.03 | 29.26 | −2.76 |
| Sepsis 20 | 32.88 | 30.35 | −2.53 |
| Sepsis 21 | 32.77 | 31.51 | −1.26 |
| Sepsis 22 | 32.19 | 30.03 | −2.16 |
| Sepsis 23 | 31.76 | 32.59 | 0.83 |
| SIRS 1 | 30.98 | 31.38 | 0.40 |
| SIRS 2 | 30.00 | 29.91 | −0.09 |
| SIRS 3 | 31.64 | 29.32 | −2.32 |
| SIRS 4 | 32.16 | 30.76 | −1.40 |
| SIRS 5 | 32.82 | 31.66 | −1.16 |
| SIRS 6 | 31.50 | 31.76 | 0.26 |
| SIRS 7 | 31.97 | 33.88 | 1.91 |
| SIRS 8 | 32.96 | 30.20 | −2.76 |
| SIRS 9 | 32.30 | 31.45 | −0.85 |
| SIRS 10 | 31.88 | 33.33 | 1.45 |
| SIRS 11 | 31.12 | 33.61 | 2.49 |
| SIRS 12 | 31.06 | 31.53 | 0.46 |
| SIRS 13 | 30.86 | 33.35 | 2.48 |
| SIRS 14 | 31.74 | 31.21 | −0.53 |
| SIRS 15 | 31.67 | 31.45 | −0.22 |
| SIRS 16 | 32.02 | 32.41 | 0.40 |
| SIRS 17 | 30.59 | 30.22 | −0.37 |
| SIRS 18 | 31.01 | 30.97 | −0.04 |
| SIRS 19 | 31.57 | 31.41 | −0.16 |

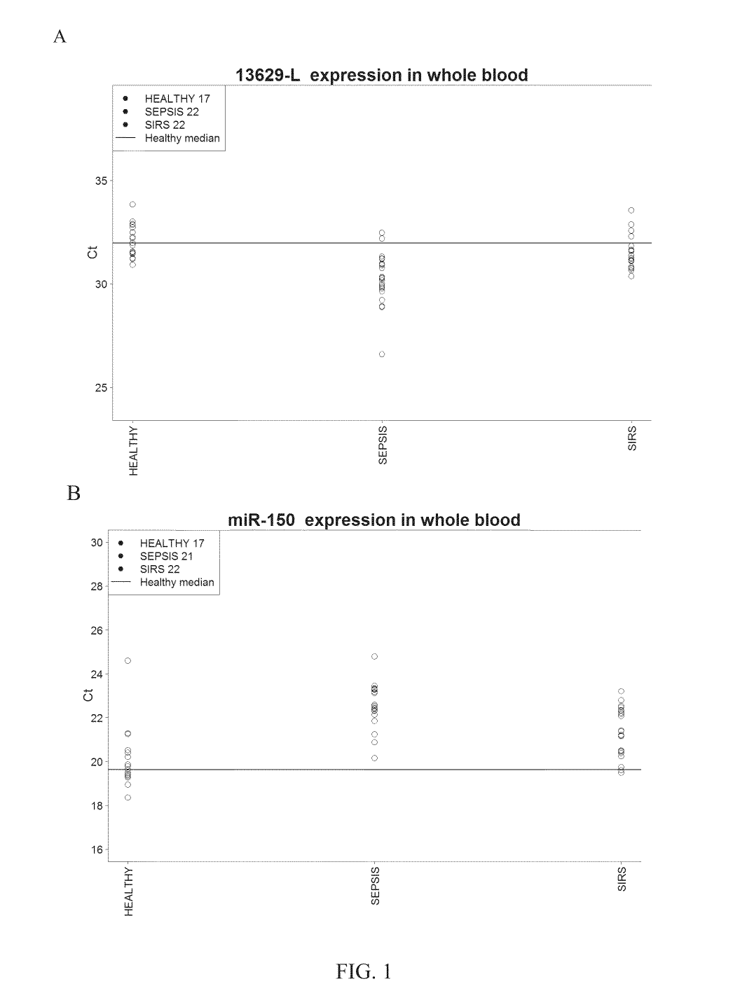

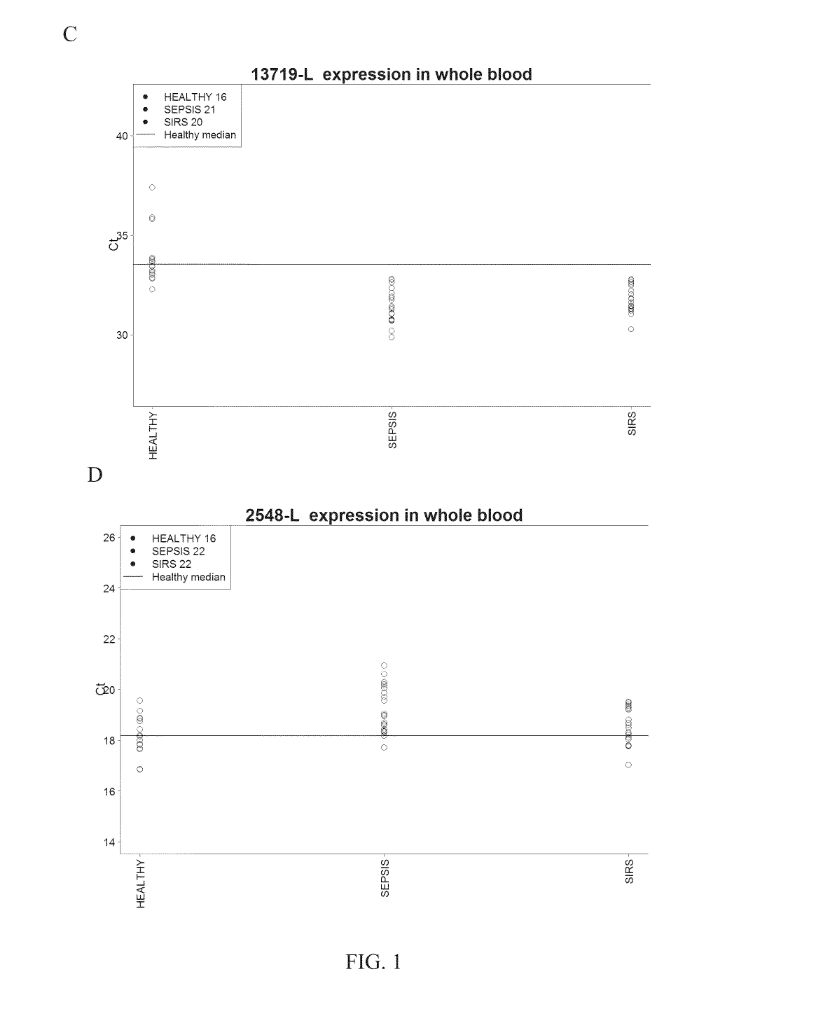

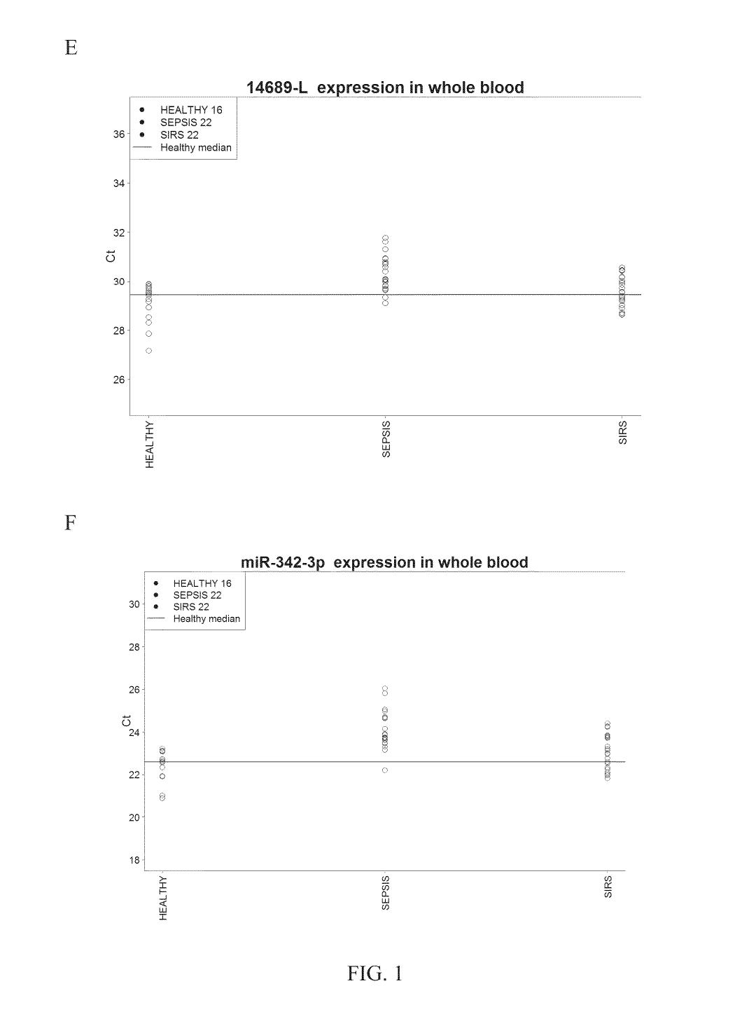

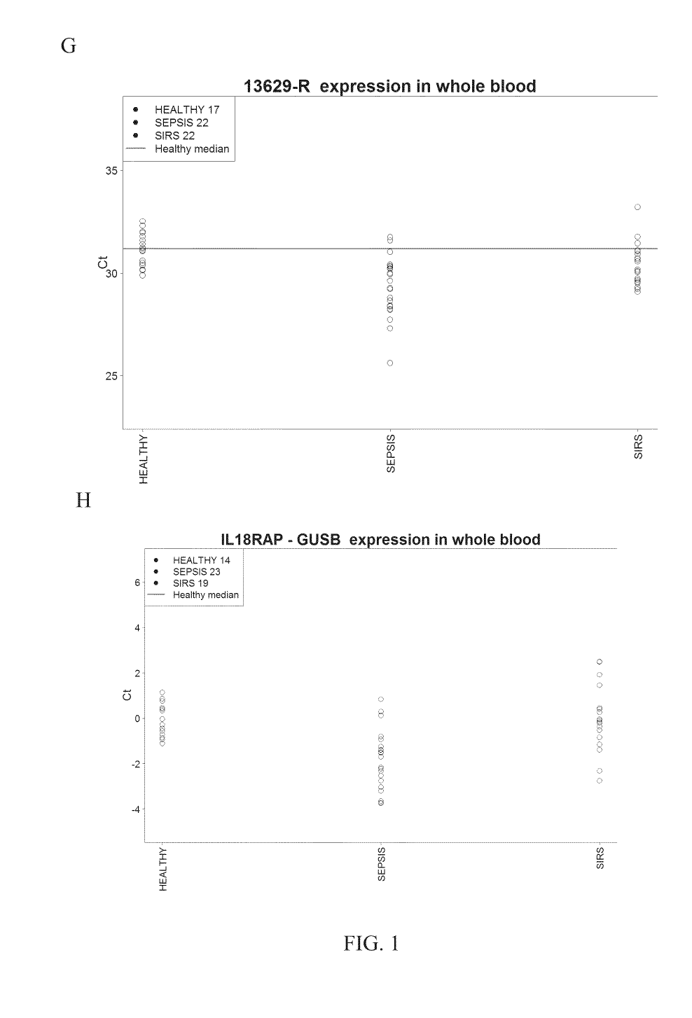

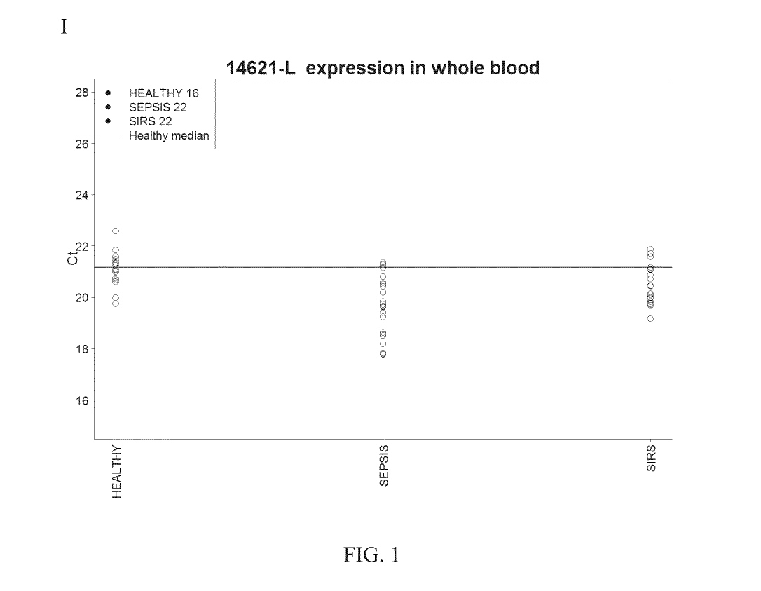

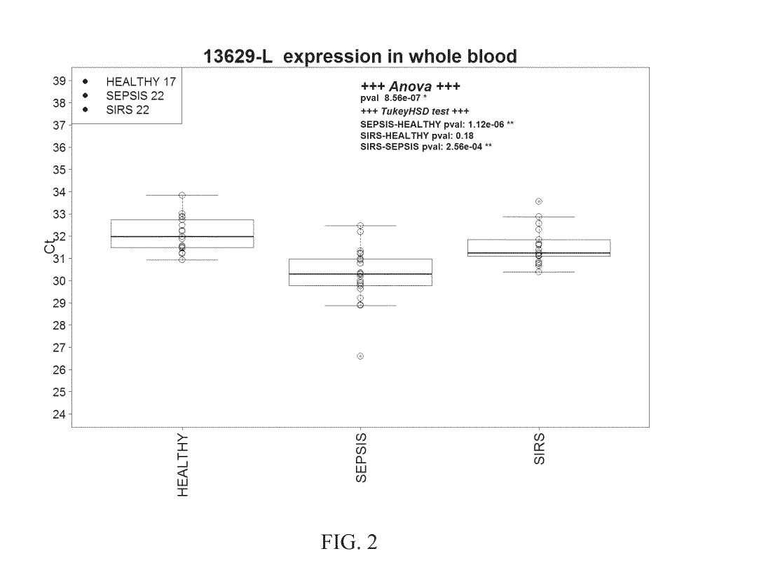

FIG. 1 shows plots of Ct values for (A) 13629-L, (B) miR-150, (C) 13719-L, (D) 2548-L, (E) 14689-L, (F) miR-342, (G) 13629-R, (H) IL18RAP, and (I) 14621-L. FIG. 2 shows an exemplary analysis of the statistical significance (Anova and Tukey's HSD test) between pairs of conditions (healthy v. sepsis, healthy v. SIRS, and sepsis v. SIRS) for 13629-L. A similar analysis was carried out for the other small RNAs. Table 3 summarizes the results of the statistical significance analysis. As noted above, probability (p) values of less than 0.05 were considered statistically significant.

TABLE 3
Statistical significance between pairs of conditions

| Small RNA | Sepsis v. healthy | Sepsis v. SIRS | SIRS v healthy |
|---|---|---|---|
| 13629-L | p < 0.001 | p < 0.001 | p = 0.18; ns |
| 13629-R | p < 0.001 | p = 0.015 | p = 0.07; ns |
| miR-150 | p < 0.001 | p = 0.0024 | p = 0.0038 |
| 13719-L | p < 0.001 | p = 0.43; ns | p < 0.001 |
| 2548-L | p = 0.001 | p = 0.032 | p = 0.37; ns |
| 14689 | p < 0.001 | p = 0.0039 | p = 0.13; ns |
| miR-342-3p | p < 0.001 | p < 0.001 | p = 0.022 |
| 14621-L | p < 0.001 | p < 0.001 | p = 0.10 |
| IL18RAP | p < 0.001 | p < 0.001 | p = 0.98; ns |

ns = not significant

As shown in FIG. 1, 13629-L, 13629-R, and 13719-L levels were higher in whole blood samples from patients with sepsis than in whole blood from healthy patients, while 2548-L, 14689-L, miR-150, and miR-342-3p levels were lower in whole blood samples from patients with sepsis than in whole blood from healthy individuals. Further, as shown in Table 3, all of the small RNAs tested showed statistically significant differences in levels between sepsis patients and healthy individuals. In addition, 13629-L, 13629-R, miR-150, 2548-L, 14689-L, and miR-342-3p showed statistically significant differences in levels between sepsis patients and SIRS patients. Finally, miR-150, 13719-L, and miR-342-3p showed statistically significant differences in levels between healthy individuals and SIRS patients.

### 5.2 Example 2

### Small RNA Combinations Improve Separation Between Sepsis, Healthy, and SIRS Patients

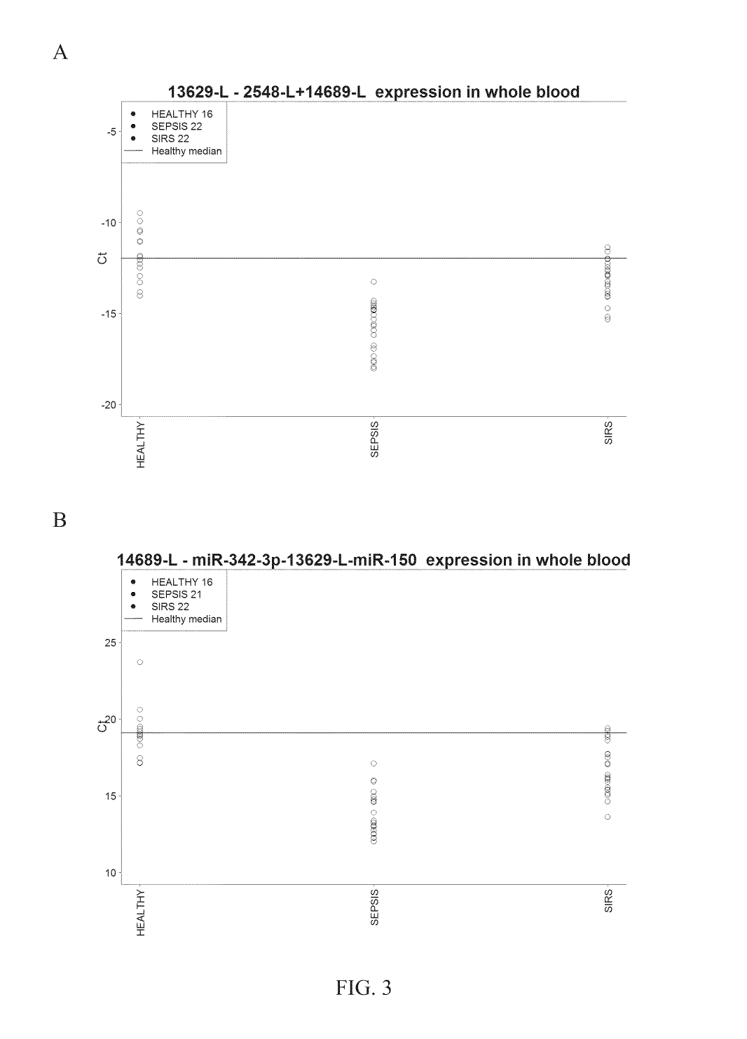

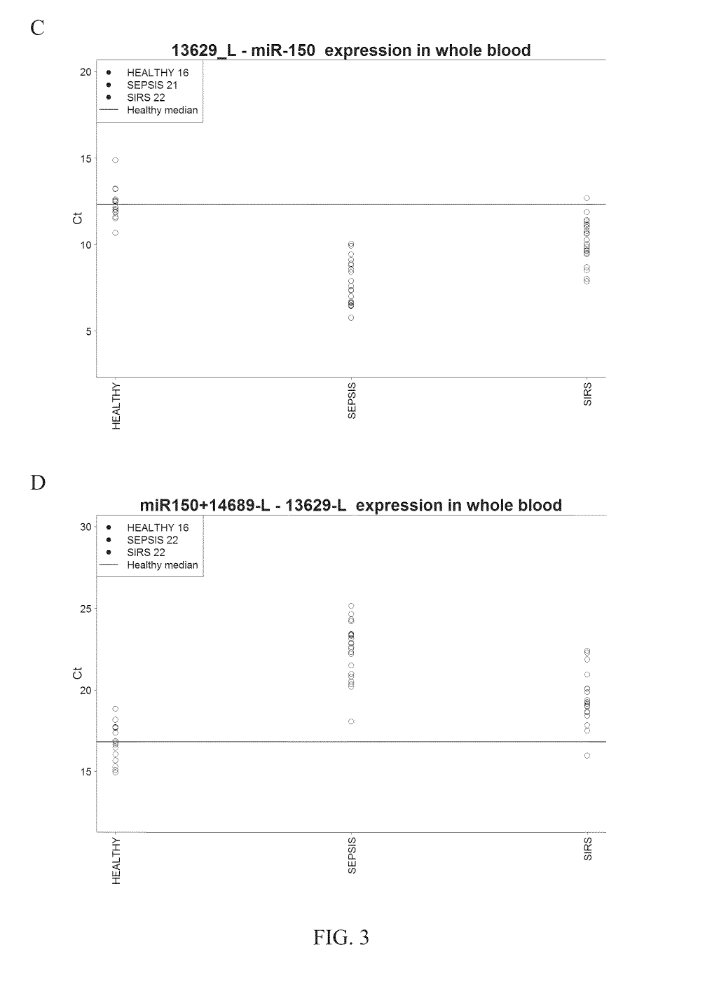

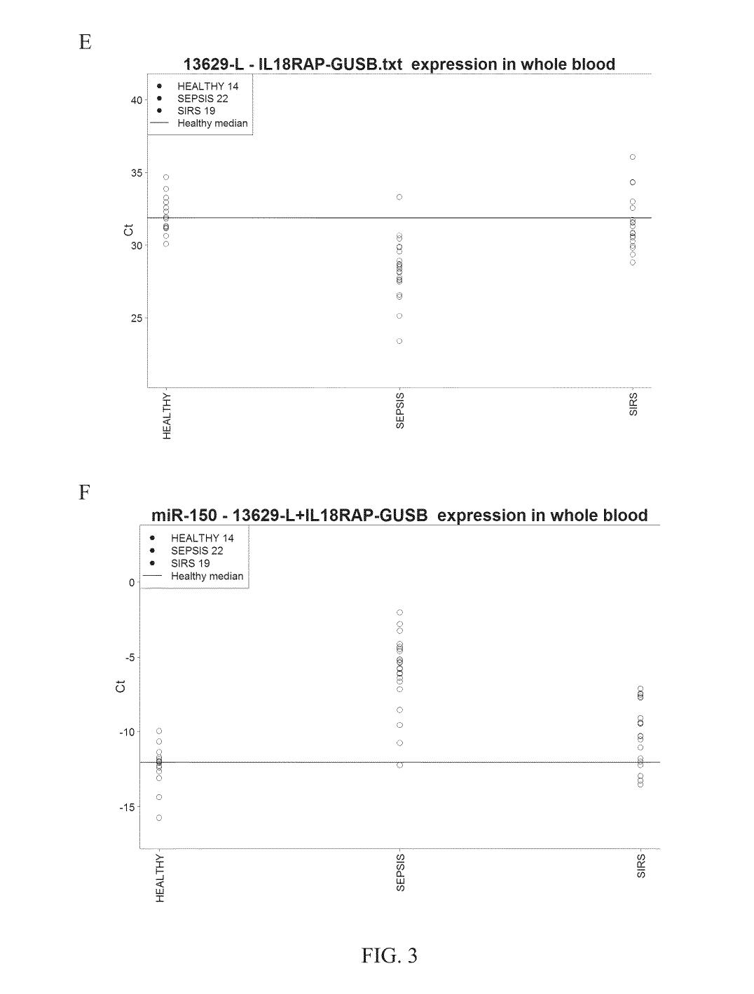

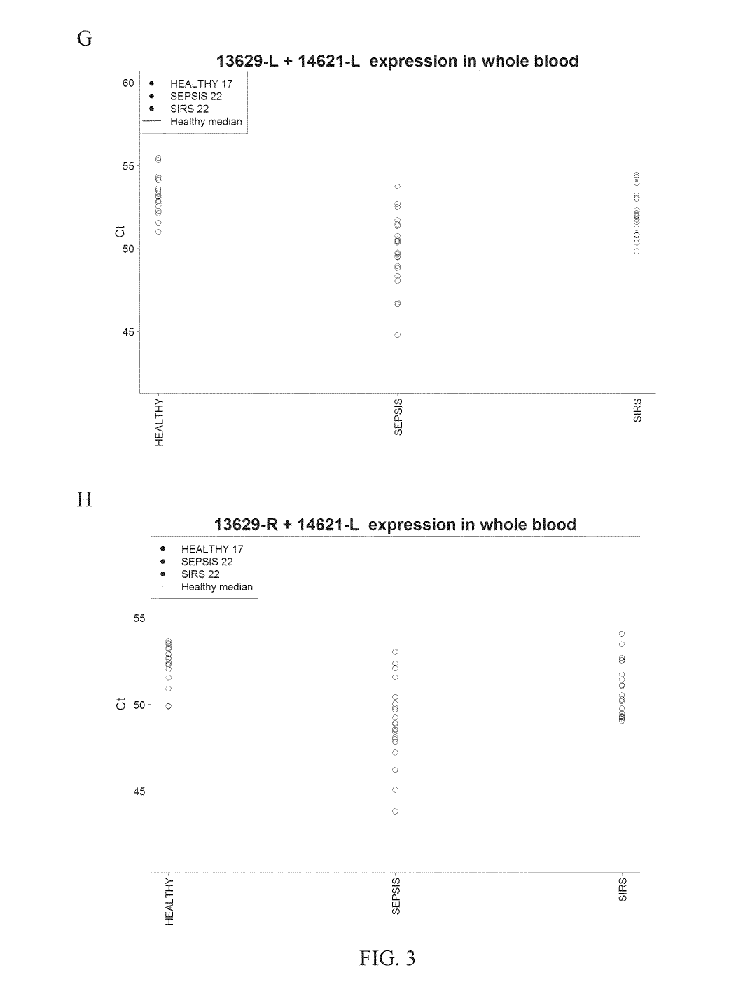

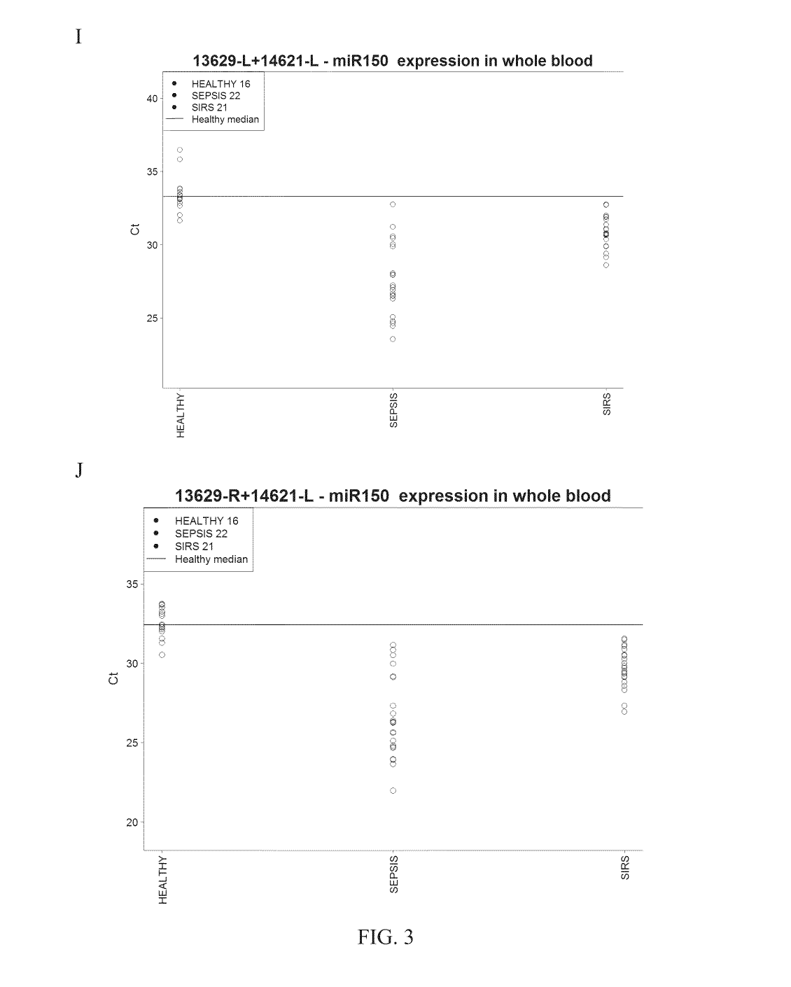

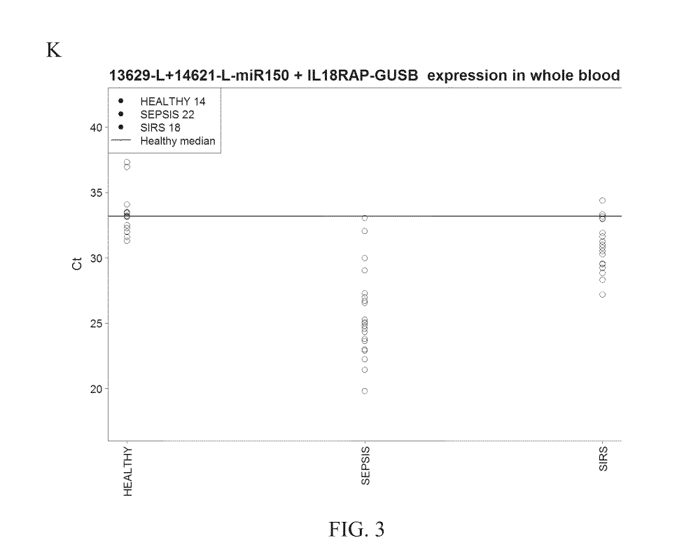

Various combinations of Ct values for 13629-L, 13719-L, miR-150, IL18RAP, 2548-L, 14689-L, 14621-L, and miR-342-3p for sepsis patients, SIRS patients, and healthy individuals were plotted and the ability of the combinations to distinguish between each pair of samples was determined using the Tukey's HSD test. FIG. 3 shows plots of (A) 13629-L−2548-L+14689-L; (B) 14689-L−miR-342-3p−13629-L−miR-150; (C) 13629-L−miR-150; (D) miR-150+14689-L−13629-L; (E) 13629-L+(IL18RAP−GUSB); (F) miR-150−13629-L+(IL18RAP−GUSB); (G) 13629-L+14621-L; (H) 13629-R+14621-L; (I) 13629-L+14621-L−miR-150; (J) 13629-R+14621-L−miR-150; and (K) 13629-L+14621-L−miR-150+(IL18RAP−GUSB).

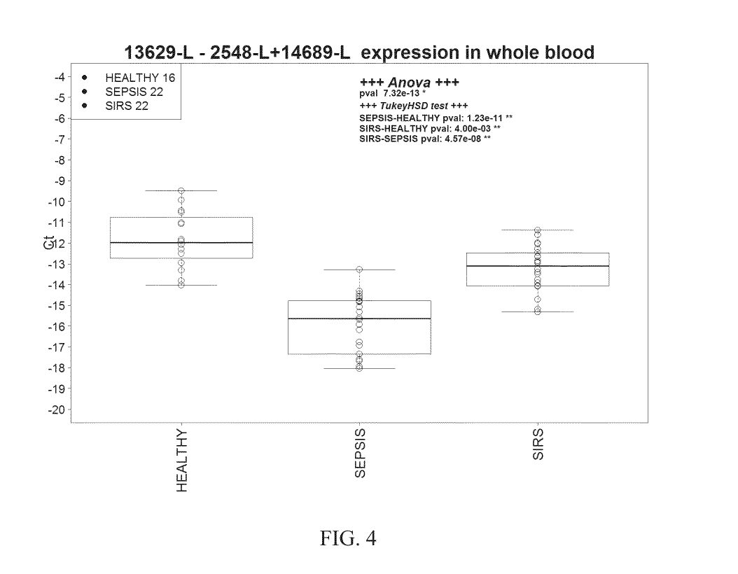

FIG. 4 shows an exemplary analysis of the data for the combination of 13629-L−2548-L+14689-L using Tukey's HSD test. A similar analysis was carried out for all of the combinations in FIG. 3. Table 4 summarizes the results of the statistical significance analysis. As noted above, probability (p) values of less than 0.05 were considered statistically significant.

TABLE 4
Statistical significance between pairs of conditions

| | Sepsis v. | Sepsis v. | SIRS v |
|---|---|---|---|
| Combination | healthy | SIRS | healthy |
| 13629-L − 2548-L + 14689-L | p < 0.001 | p < 0.001 | p = 0.004 |
| 14689-L − miR-342-3p − | p < 0.001 | p < 0.001 | p < 0.001 |
| 13629-L − miR-150 | | | |
| 13629-L − miR-150 | p < 0.001 | p < 0.001 | p < 0.001 |
| miR-150 + 14689-L − 13629-L | p < 0.001 | p < 0.001 | p < 0.001 |
| 13629-L + (IL18RAP − GUSB) | p < 0.001 | p < 0.001 | p = 0.67; ns |
| miR-150 − 13629-L + (IL18RAP − | p < 0.001 | p < 0.001 | p = 0.016 |
| GUSB) | | | |
| 13629-L + 14621-L | p < 0.001 | p < 0.001 | p = 0.07; ns |
| 13629-R + 14621-L | p < 0.001 | p = 0.001 | p = 0.034 |
| 13629-L + 14621-L − miR-150 | p < 0.001 | p < 0.001 | p < 0.001 |
| 13629-R + 14621-L − miR-150 | p < 0.001 | p < 0.001 | p < 0.001 |
| 13629-L + 14621-L − miR-150 + | p < 0.001 | p < 0.001 | p = 0.022 |
| (IL18RAP − GUSB) | | | |

As shown in FIG. 3, the tested combinations of small RNAs increased the separation between healthy individuals, sepsis patients, and SIRS patients. As shown in Table 4, all of the combinations tested showed statistically significant differences in levels between sepsis patients v. healthy individuals and sepsis patients v. SIRS patients. Thus, all of the combinations are able to distinguish sepsis from SIRS, and sepsis from healthy individuals. Further, all but two of the combinations tested showed statistically significant differences in levels between SIRS patients v. healthy individuals.

### 5.3 Example 3

### Small RNA Combinations Improve Detection Sensitivity and Specificity

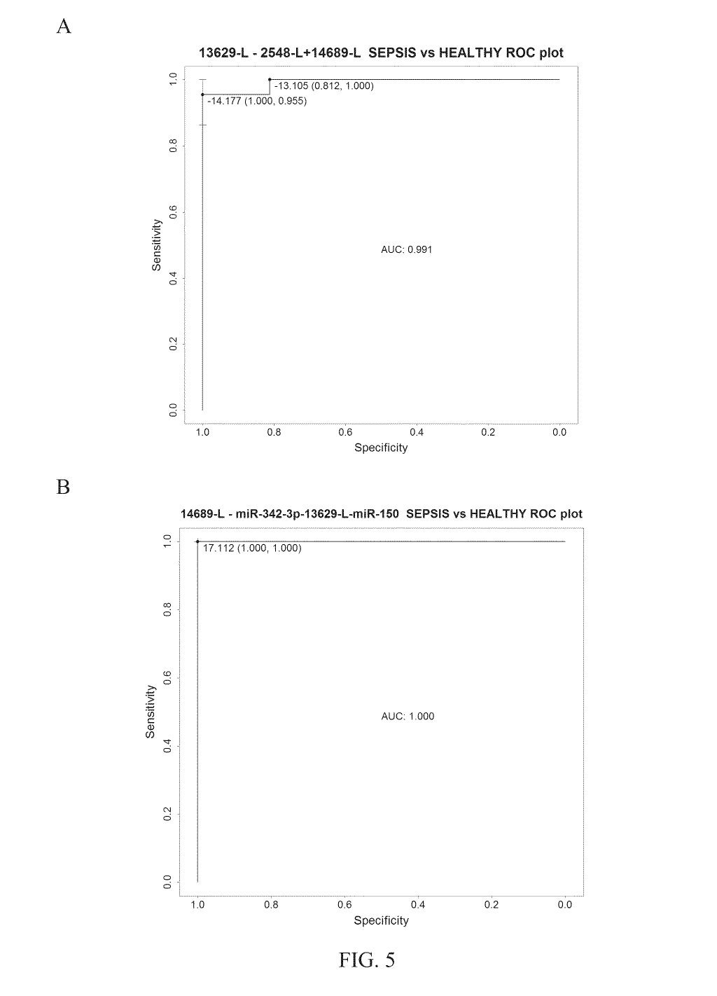

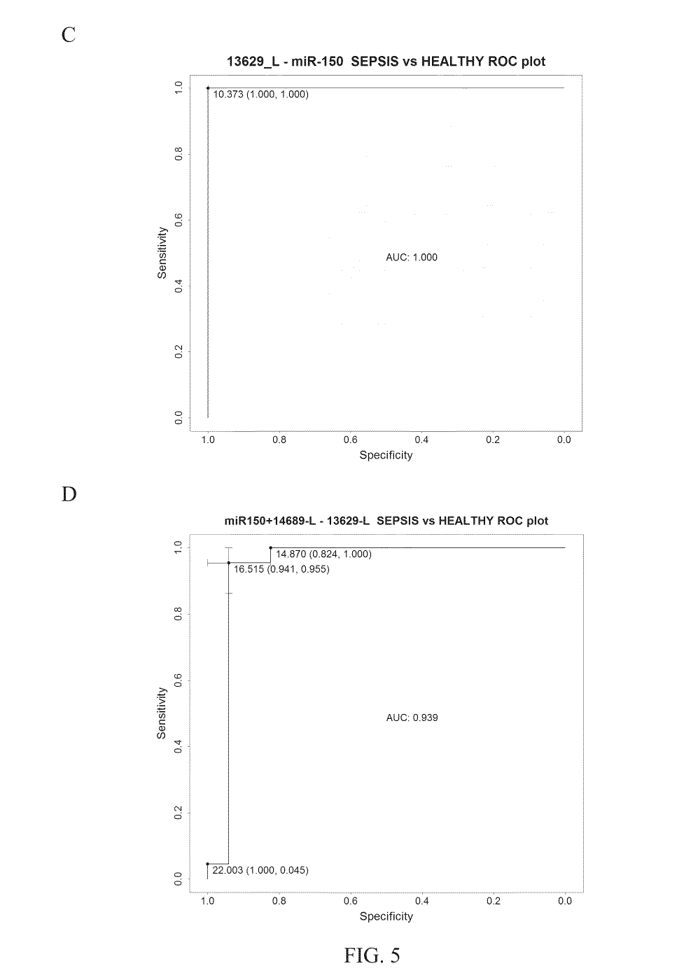

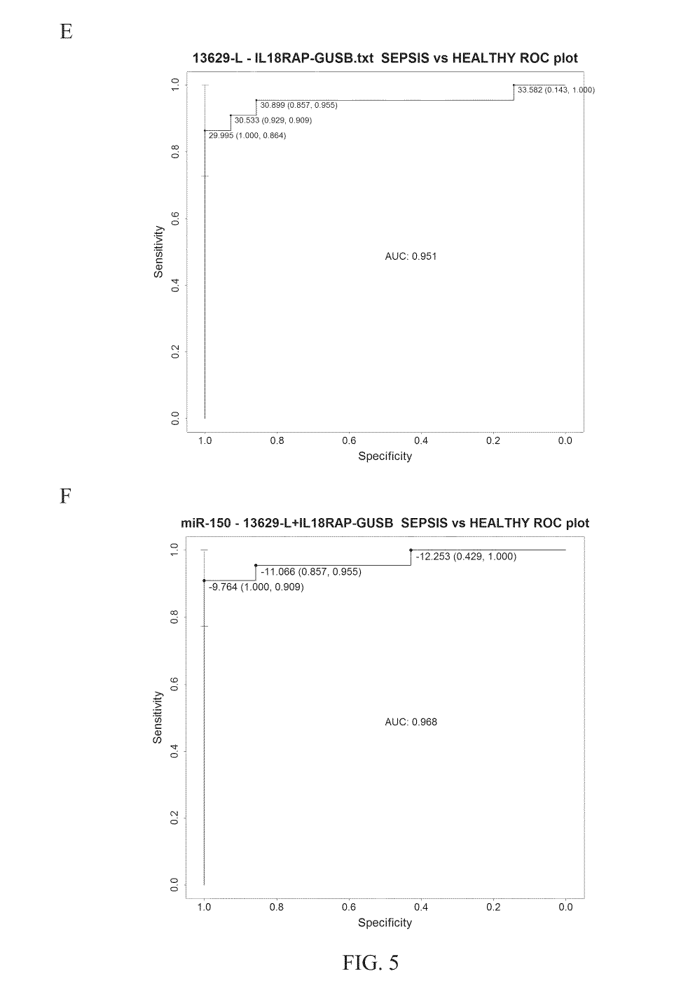

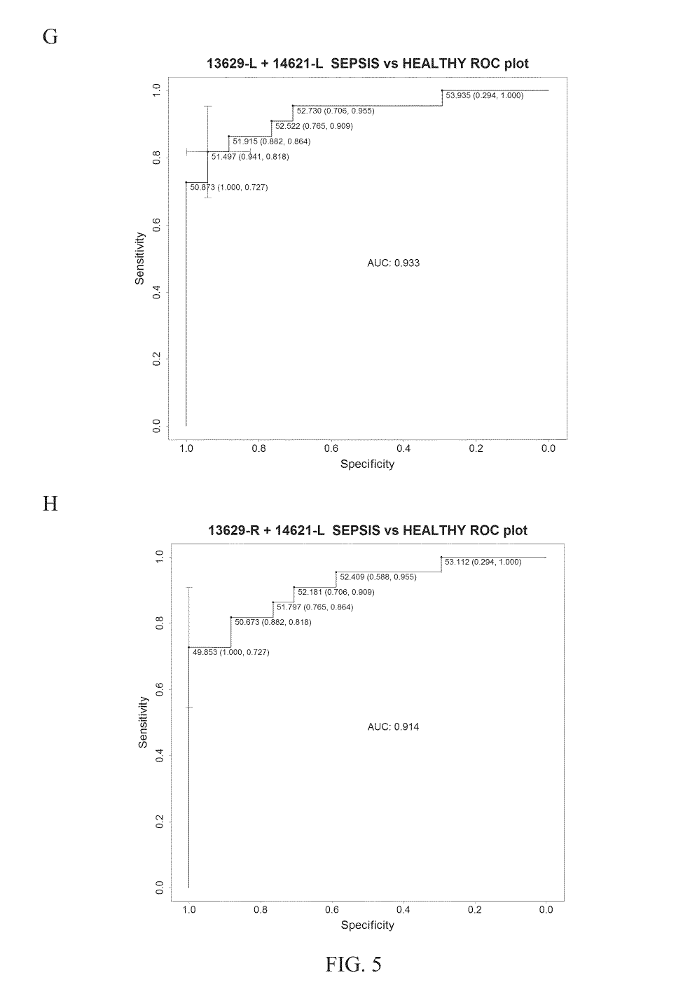

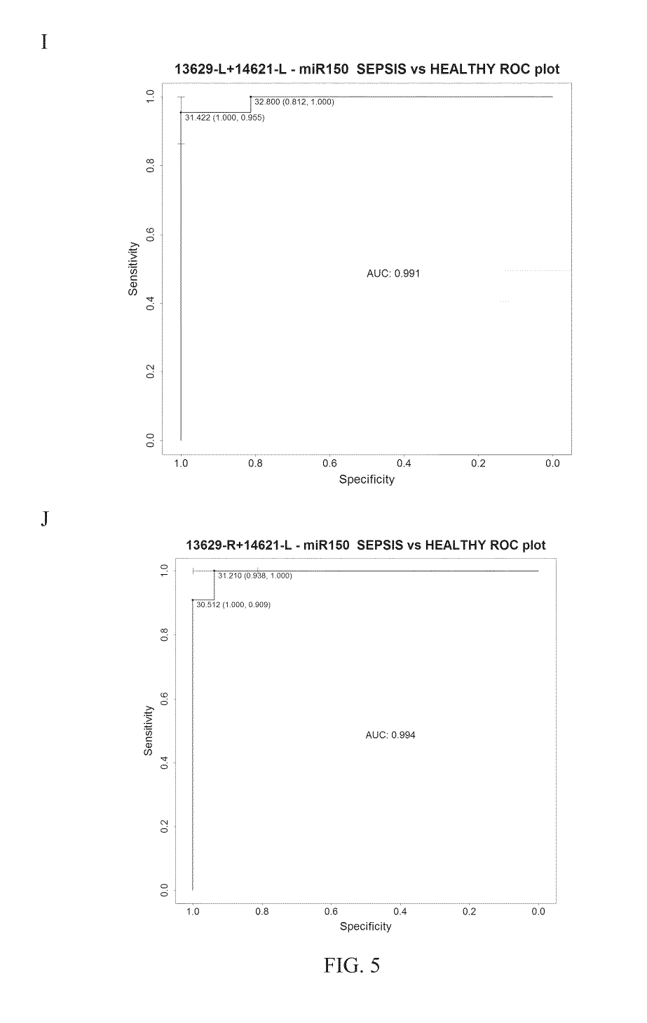

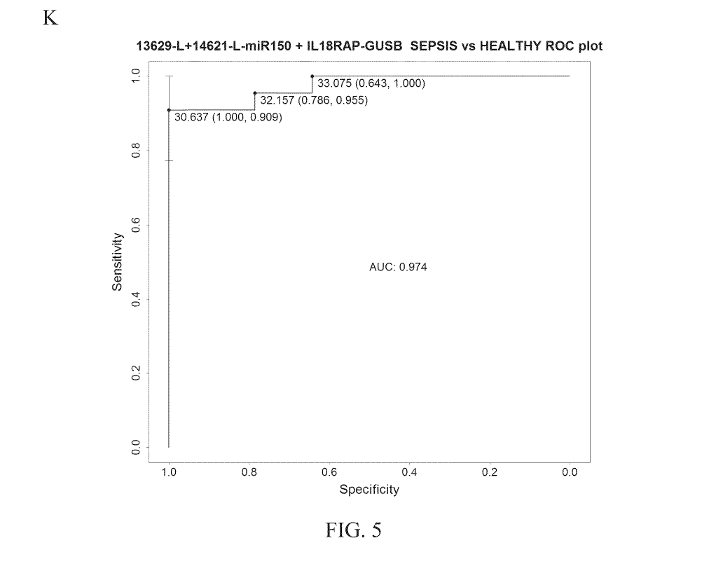

Receiver operating characteristic (ROC) curves were plotted for certain small RNA combinations to determine the specificity and sensitivity of the small RNA combinations for distinguishing between pairs of conditions (i.e., healthy vs. sepsis, healthy vs. SIRS, sepsis vs. SIRS), using the pROC package. See, e.g., Xavier Robin, Natacha Turck, Alexandre Hainard, Natalia Tiberti, Frédérique Lisacek, Jean-Charles Sanchez and Markus Müller (2011). pROC: an open-source package for R and S+ to analyze and compare ROC curves. BMC Bioinformatics, 12, p. 77. DOI: 10.1186/1471-2105-12-77. FIG. 5 shows ROC plots for sepsis versus healthy, for the combinations of (A) 13629-L−2548-L+14689-L; (B) 14689-L−miR-342-3p−13629-L−miR-150; (C) 13629-L−miR-150; (D) miR-150+14689-L−13629-L; (E) 13629+(IL18RAP-GUSB); (F) miR-150−13629-L+(IL18RAP−GUSB); (G) 13629-L+14621-L; (H) 13629-R+14621-L; (I) 13629-L+14621-L−miR-150; (J) 13629-R+14621-L−miR-150; and (K) 13629-L+14621-L−miR-150+(IL18RAP−GUSB).

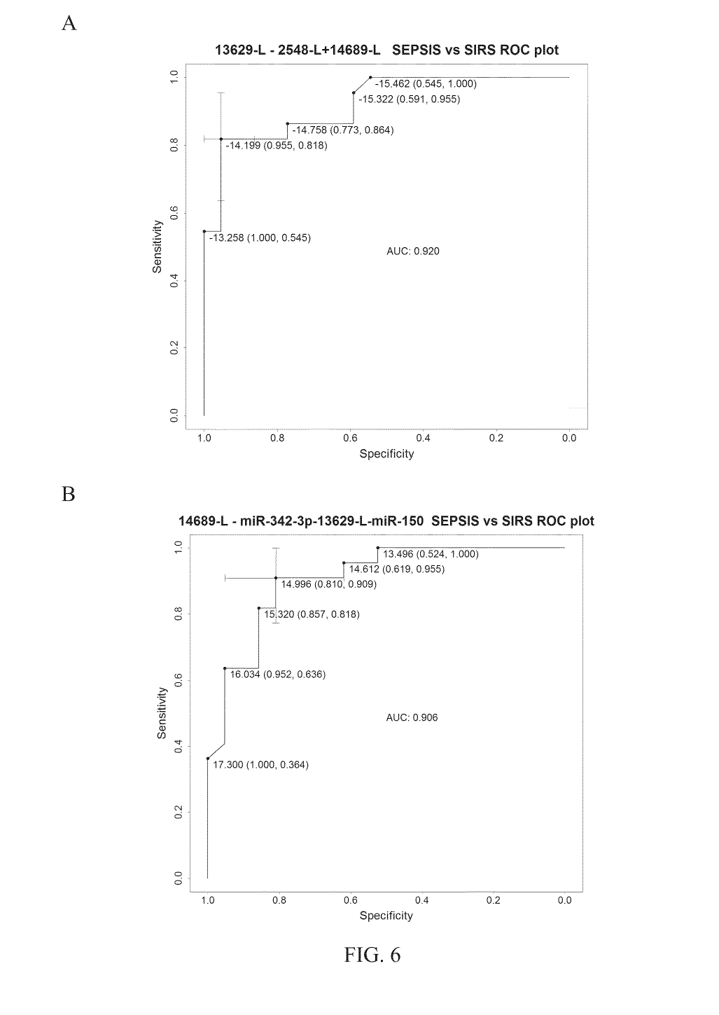

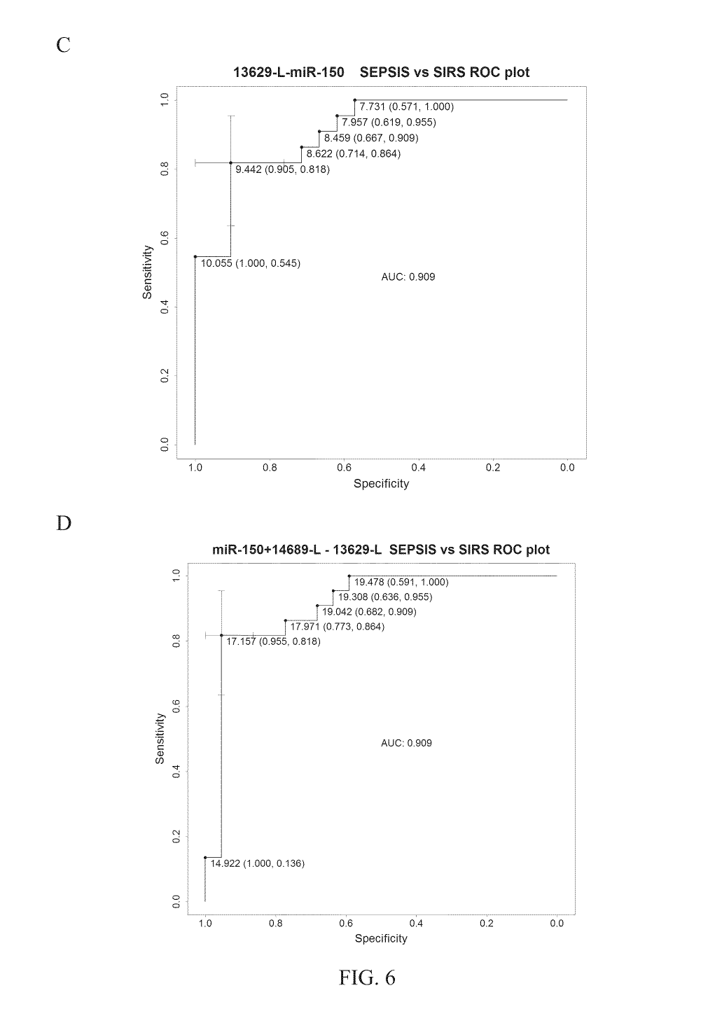

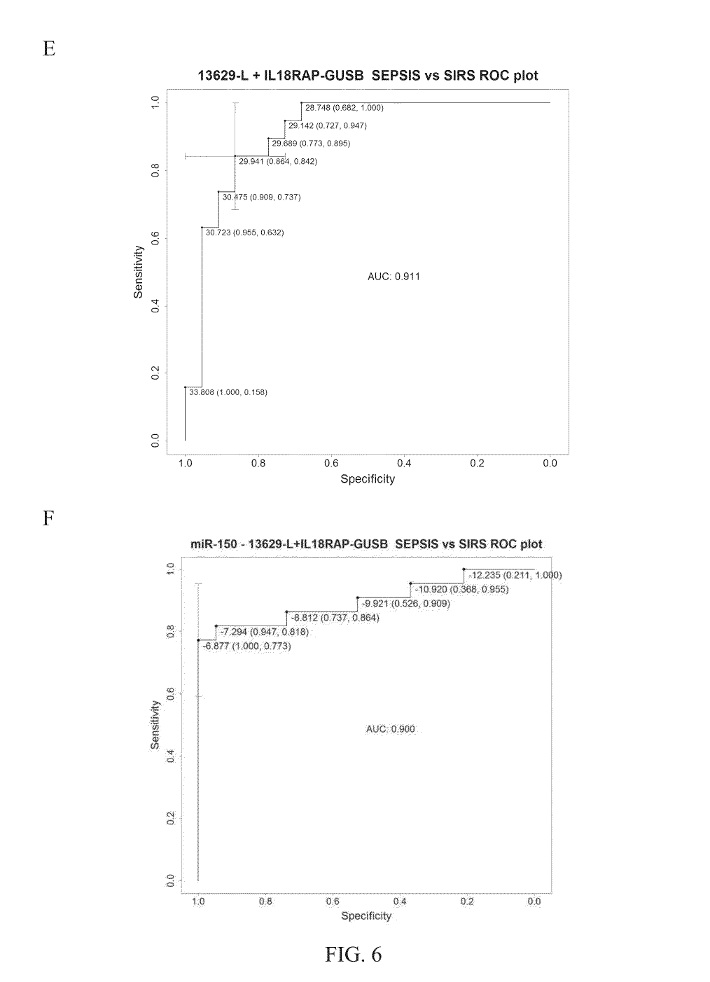

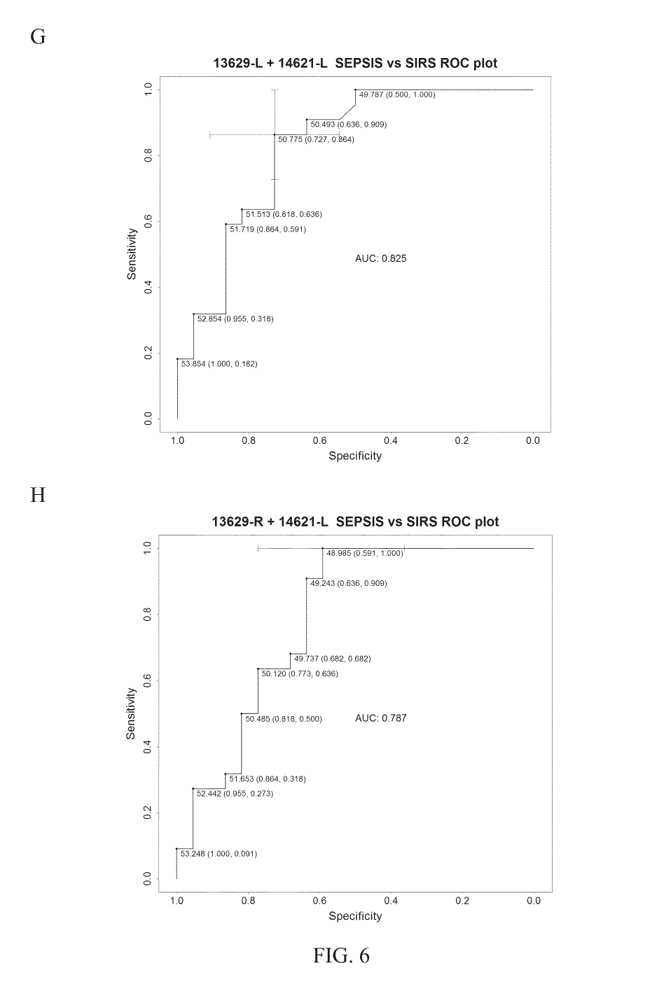

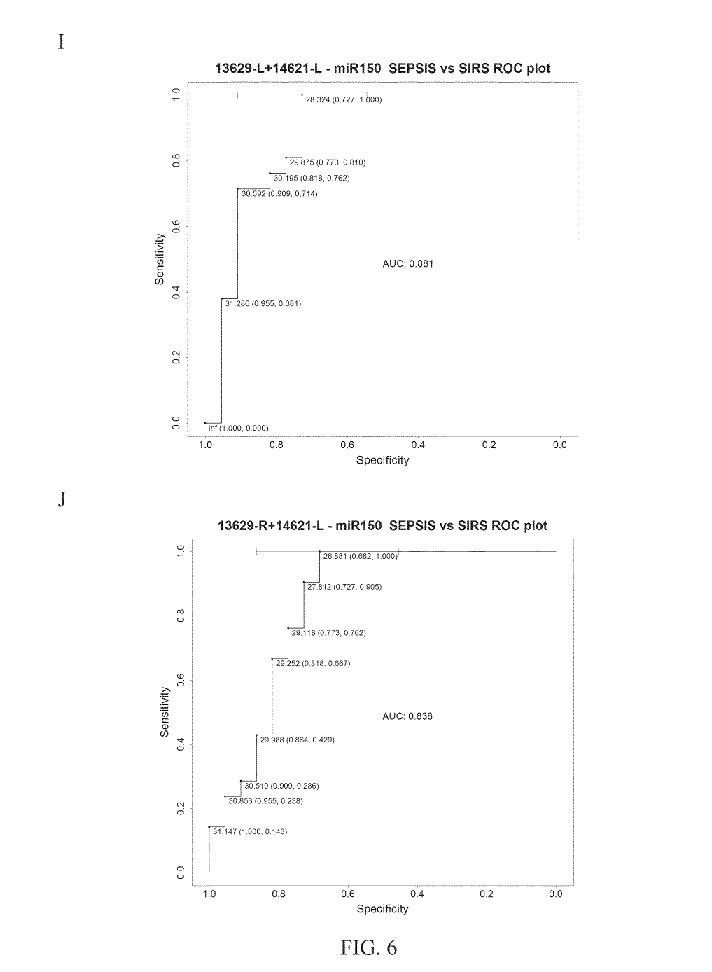

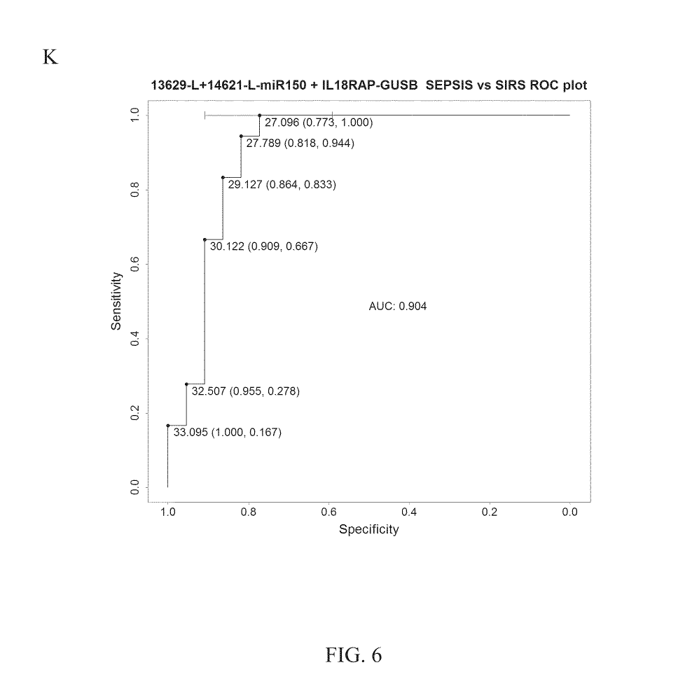

FIG. 6 shows ROC plots for sepsis versus SIRS, for the combinations of (A) 13629-L−2548-L+14689-L; (B) 14689-L−miR-342-3p−13629-L−miR-150; (C) 13629-L−miR-150; (D) miR-150+14689-L−13629-L; (E) 13629+(IL18RAP-GUSB); (F) miR-150−13629-L+(IL18RAP GUSB); (G) 13629-L+14621-L; (H) 13629-R+14621-L; (I) 13629-L+14621-L−miR-150; (J) 13629-R+14621-L−miR-150; and (K) 13629-L+14621-L−miR-150+(IL18RAP−GUSB).

Table 5 shows certain sensitivity and specificity results for the combinations at a particular Ct cutoff.

TABLE 5
Sensitivity and specificity of small RNA combinations

| | Sepsis v. healthy | | | Sepsis v. SIRS | | |
|---|---|---|---|---|---|---|
| | Ct | | | Ct | | |
| Combination | cutoff | sens | spec | cutoff | sens | spec |
| 13629-L − 2548-L + 14689-L | −14.2 | 95.5% | 100% | −14.2 | 81.8% | 95.5% |
| 14689-L − miR-342-3p − 13629-L − | 17.1 | 100% | 100% | 15.0 | 90.9% | 81% |
| miR-150 | | | | | | |
| 13629-L − miR-150 | 10.4 | 100% | 100% | 9.4 | 81.8% | 90.5% |
| miR-150 + 14689-L − 13629-L | 16.5 | 95.5% | 94.1% | 17.2 | 81.8% | 95.5% |
| 13629 + (IL18RAP-GUSB) | 30 | 86.4% | 100% | 29.9 | 84.2% | 86.4% |
| miR-150 − 13629-L + (IL18RAP-GUSB) | −9.8 | 90.9% | 100% | −6.9 | 77.3% | 100% |
| 13629-L + 14621-L | 51.5 | 81.8% | 94.1% | 50.8 | 86.4% | 72.7% |
| 13629-R + 14621-L | 49.9 | 72.7% | 100% | 49.0 | 100% | 59.1% |
| 13629-L + 14621-L − miR-150 | 31.4 | 95.5% | 100% | 28.3 | 100% | 72.7% |
| 13629-R + 14621-L − miR-150 | 31.2 | 100% | 93.8% | 26.9 | 100% | 68.2% |
| 13629-L + 14621-L − miR-150 + | 30.6 | 90.9% | 100% | 27.1 | 100% | 77.3% |
| (IL18RAP − GUSB) | | | | | | |

Four of the tested combinations were able to distinguish between samples from sepsis patients and samples from healthy individuals with >95% sensitivity and 100% specificity. Three additional combinations were able to distinguish between samples from sepsis patients and samples from healthy individuals with 100% specificity, and a sensitivity of >85%. The remaining combinations were able to distinguish between samples from sepsis patients and samples from healthy individuals with >72% sensitivity and >93% specificity.

In addition, the tested combinations were able to distinguish between samples from sepsis patients and samples from SIRS patients with at least 80% sensitivity and, for all but two of the combinations, at least 72% specificity.

### 5.4 Example 4

### Small RNA Combination Distinguishes Between Sepsis Patients and Non-Sepsis Patients with Infections or Inflammation

Whole blood samples from (1) five healthy individuals of both sexes ranging in age from 18 to 64; (2) 24 patients of both sexes with bacterial infections (including, but not limited to, cellulitis (n=12), pyelonephritis (n=9), urinary tract infections (n=5), and chest infection (n=2), due to various organisms, including, but not limited to, E. coli, Staphylococcus, Streptococcus, and Pseudomonas), ranging in age from 20 to 74; and (3) 34 patients of both sexes with inflammatory conditions (including, but not limited to, chest pain (n=20), injury (n=4), abdominal pain (n=3), painful leg (n=2), and collapse (n=2)) ranging in age from 18 to 85.

RNA was prepared from the whole blood samples as described in Example 1.

13629-L and miR-150 levels were detected by qRT-PCR using Exiqon custom LNA primers (Exiqon, Vedbaek, DK), according to the manufacturer's instructions. Experiments were performed in triplicate wells. No normalization was applied.

TABLE 6
Average Ct values for 13629-L and miR-150

| | Ct | | 13629 − |
|---|---|---|---|
| Patient | 13629-L | Ct miR-150 | miR-150 |
| Healthy 1 | 32.43 | 21.84 | 10.59 |
| Healthy 2 | 32.37 | 21.94 | 10.43 |
| Healthy 3 | 30.44 | 24.19 | 6.25 |
| Healthy 4 | 33.82 | 23.61 | 10.21 |
| Healthy 5 | 35.99 | 24.275 | 11.715 |
| Infectious 1 | 33.47 | 21.02 | 12.45 |
| Infectious 2 | 33.495 | 23.52 | 9.975 |
| Infectious 3 | 34.6 | 22.335 | 12.265 |
| Infectious 4 | 32.225 | 20.935 | 11.29 |
| Infectious 5 | 33.75 | 20.295 | 13.455 |
| Infectious 6 | 32.915 | 19.62 | 13.295 |
| Infectious 7 | 30.95 | 20.945 | 10.005 |
| Infectious 8 | 30.64 | 21.935 | 8.705 |
| Infectious 9 | 33.675 | 22.77 | 10.905 |
| Infectious 10 | 31.44 | 16.825 | 14.615 |
| Infectious 11 | 32.19 | 20.34 | 11.85 |
| Infectious 12 | 32.76 | 20.58 | 12.18 |
| Infectious 13 | 33.65 | 21.375 | 12.275 |
| Infectious 14 | 32.71 | 19.13 | 13.58 |
| Infectious 15 | 31.55 | 20.385 | 11.165 |
| Infectious 16 | 32.98 | 19.945 | 13.035 |
| Infectious 17 | 31.595 | 19.875 | 11.72 |
| Infectious 18 | 32.22 | 20.01 | 12.21 |
| Infectious 19 | 33.11 | 18.91 | 14.2 |
| Infectious 20 | 31.81 | 20.9 | 10.91 |
| Infectious 21 | 31.875 | 19.385 | 12.49 |
| Infectious 22 | 32.215 | 19.195 | 13.02 |
| Infectious 23 | 30.44 | 20.08 | 10.36 |
| Infectious 24 | 32.795 | 23.49 | 9.305 |
| Inflammatory 1 | 33.55 | 23.25 | 10.3 |
| Inflammatory 2 | 34.6 | 24.44 | 10.16 |
| Inflammatory 3 | 33.65 | 23.395 | 10.255 |
| Inflammatory 4 | 32.155 | 22.9 | 9.255 |
| Inflammatory 5 | 36.98 | 22.505 | 14.475 |
| Inflammatory 6 | 35.915 | 21.955 | 13.96 |
| Inflammatory 7 | 34.695 | 23.005 | 11.69 |
| Inflammatory 8 | 32.785 | 20 | 12.785 |
| Inflammatory 9 | 32.79 | 19.555 | 13.235 |
| Inflammatory 10 | 33.53 | 20.06 | 13.47 |
| Inflammatory 11 | 27.075 | 15.53 | 11.545 |
| Inflammatory 12 | 32.485 | 20.03 | 12.455 |
| Inflammatory 13 | 31.17 | 19.98 | 11.19 |
| Inflammatory 14 | 32.66 | 20.29 | 12.37 |
| Inflammatory 15 | 32.19 | 20.66 | 11.53 |
| Inflammatory 16 | 31.605 | 21.035 | 10.57 |
| Inflammatory 17 | 34.02 | 19.83 | 14.19 |
| Inflammatory 18 | 34.295 | 21.7 | 12.595 |
| Inflammatory 19 | 34.39 | 20.33 | 14.06 |
| Inflammatory 20 | 32.18 | 20.89 | 11.29 |
| Inflammatory 21 | 32.99 | 18.625 | 14.365 |
| Inflammatory 22 | 31.5 | 18.98 | 12.52 |
| Inflammatory 23 | 31.38 | 19.025 | 12.355 |
| Inflammatory 24 | 33.05 | 20.325 | 12.725 |
| Inflammatory 25 | 31.82 | 18.85 | 12.97 |
| Inflammatory 26 | 33.23 | 19.195 | 14.035 |
| Inflammatory 27 | 31.895 | 19.28 | 12.615 |
| Inflammatory 28 | 31.59 | 18.065 | 13.525 |
| Inflammatory 29 | 32.745 | 18.66 | 14.085 |
| Inflammatory 30 | 31.185 | 18.535 | 12.65 |
| Inflammatory 31 | 31.095 | 18.575 | 12.52 |
| Inflammatory 32 | 34.035 | 19.715 | 14.32 |
| Inflammatory 33 | 33.09 | 18.935 | 14.155 |
| Inflammatory 34 | 35.97 | 19.6 | 16.37 |

The Ct values for miR-150 in whole blood or PBMC for various patients were subtracted from the Ct values for 13629-L in those patients. The patient populations used in this analysis included the sepsis patients, SIRS patients, and healthy individuals shown above in Table 1, and the infectious and inflammatory patients shown above in Table 6. The results were plotted and the ability of the combination to distinguish between each pair of conditions was determined using the Tukey's HSD test.

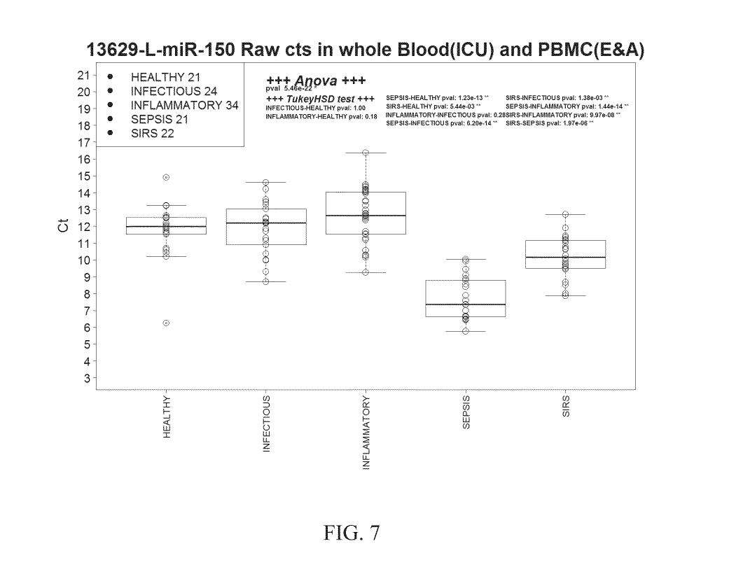

FIG. 7 shows the plot of 13629-L−miR-150, and an analysis of the data using Tukey's HSD test. Table 7 summarizes the results of the statistical significance analysis. As noted above, probability (p) values of less than 0.05 were considered statistically significant.

TABLE 7
Statistical significance between pairs of conditions

| | 13629-L − |
|---|---|
| Condition | miR-150 |
| Infectious v. healthy | p = 1; ns |
| Inflammatory v. healthy | p = 0.18; ns |
| Sepsis v. healthy | p < 0.001 |
| SIRS v. healthy | p = 0.005 |
| Inflammatory v. infectious | p = 0.28; ns |
| Sepsis v. infectious | p < 0.001 |
| SIRS v. infectious | p = 0.001 |
| Sepsis v. inflammatory | p < 0.001 |
| SIRS v. inflammatory | p < 0.001 |
| Sepsis v. SIRS | p < 0.001 |

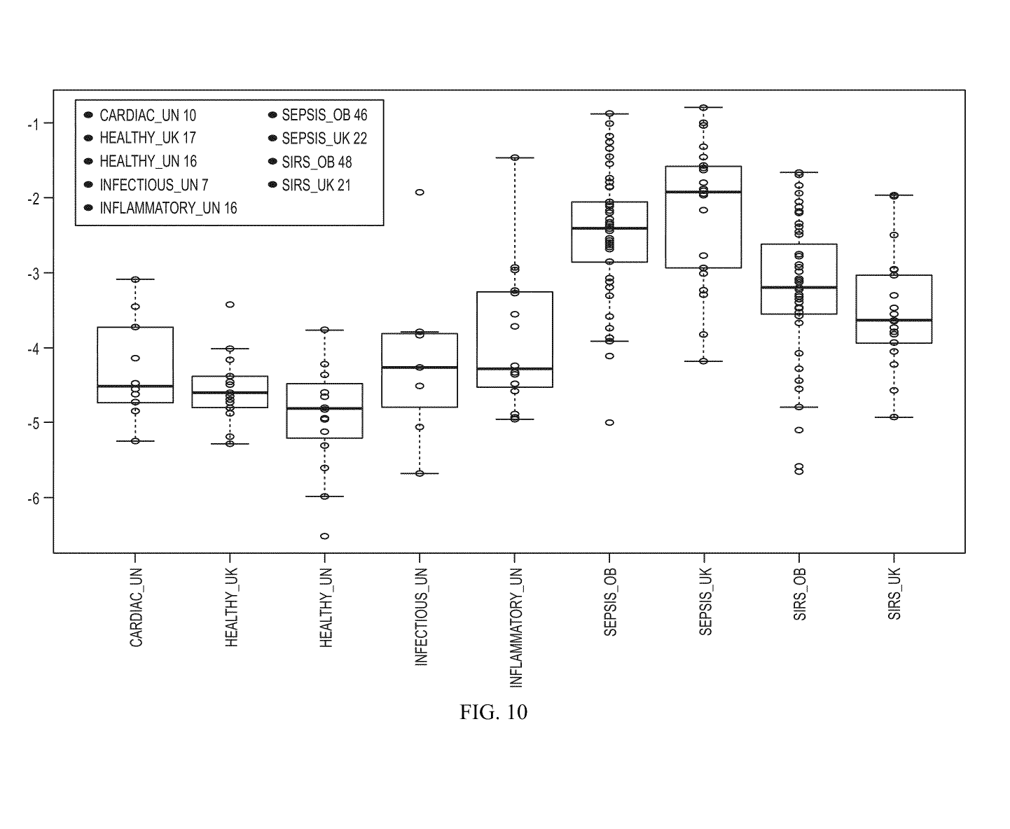

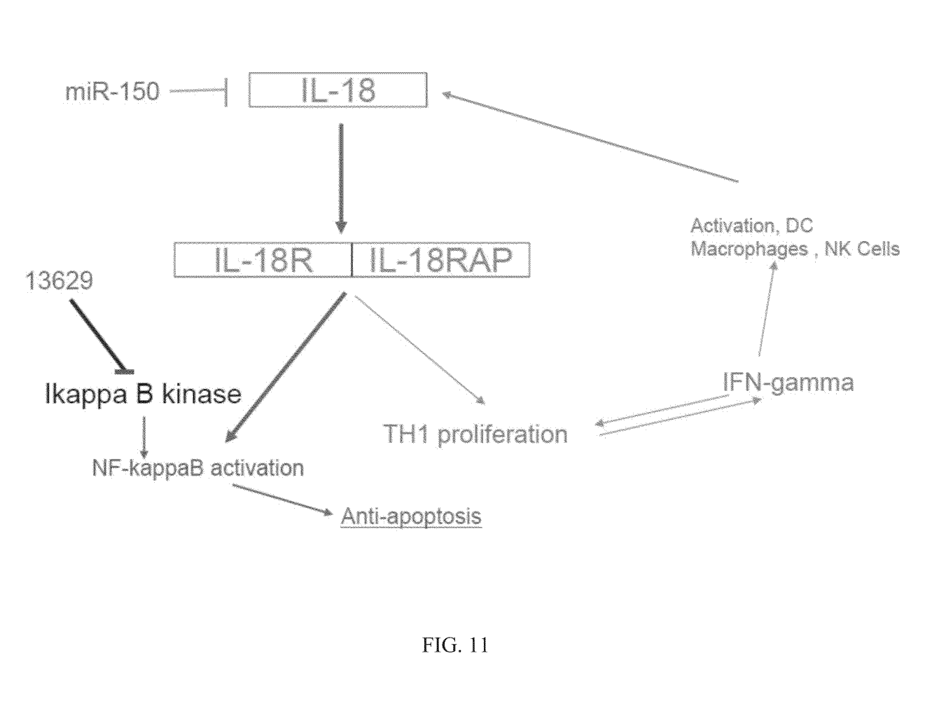

As shown in FIG. 7 and Table 7, the tested combination of 13629-L and miR-150 was able to distinguish between sepsis patients and non-sepsis patients. Without intending to be bound by any particular theory, it is hypothesized that miR-150 may target IL-18 expression and 13629 may target IκB expression. In sepsis, miR-150 levels are reduced and 13629 levels are increased, leading to an increase in IL-18 expression and a decrease in IκB expression, both of which may result in increased NF-κB activity. FIG. 11 shows a diagram of the hypothetical model.

### 5.5 Example 5

### Small RNA Combination Distinguishes Between Sepsis Patients and Non-Sepsis Patients with Cardiac Conditions

The combination of 13629-L and miR-150 was tested in an additional control group, patients admitted to the hospital with cardiac conditions. The patient samples used in the study included samples from four patients with ischaemic heart disease, four patients with myocardial infarction, four patients with unstable angina, two patients with acute coronary syndrome, and one patient with atrial fibrillation. One patient was 17 years old, with the rest ranging in age from 65 to 73. The group included 11 men and four women.

In this experiment, a linear discriminant model was used for the analysis. The coefficients of linear discriminants were determined to be −0.5640718 for 13629-L and 0.7995699 for miR-150 for this experiment. Thus, the LDA score=−0.5670718*Ct_{—}13629-L+0.7995699*Ct_miR-150.

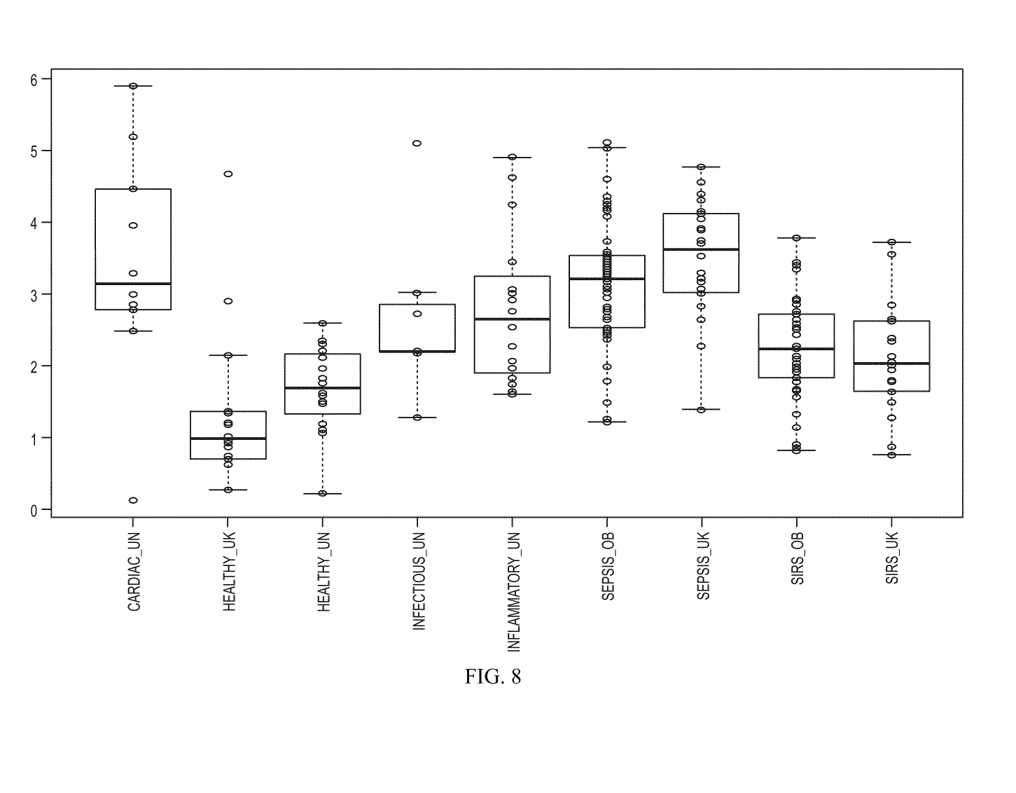

As shown in FIG. 8, the patients with cardiac conditions were difficult to distinguish from the sepsis patients in this experiment. The healthy patients and patients with infectious disease, inflammatory conditions, or SIRS were still distinguishable from the sepsis patients.

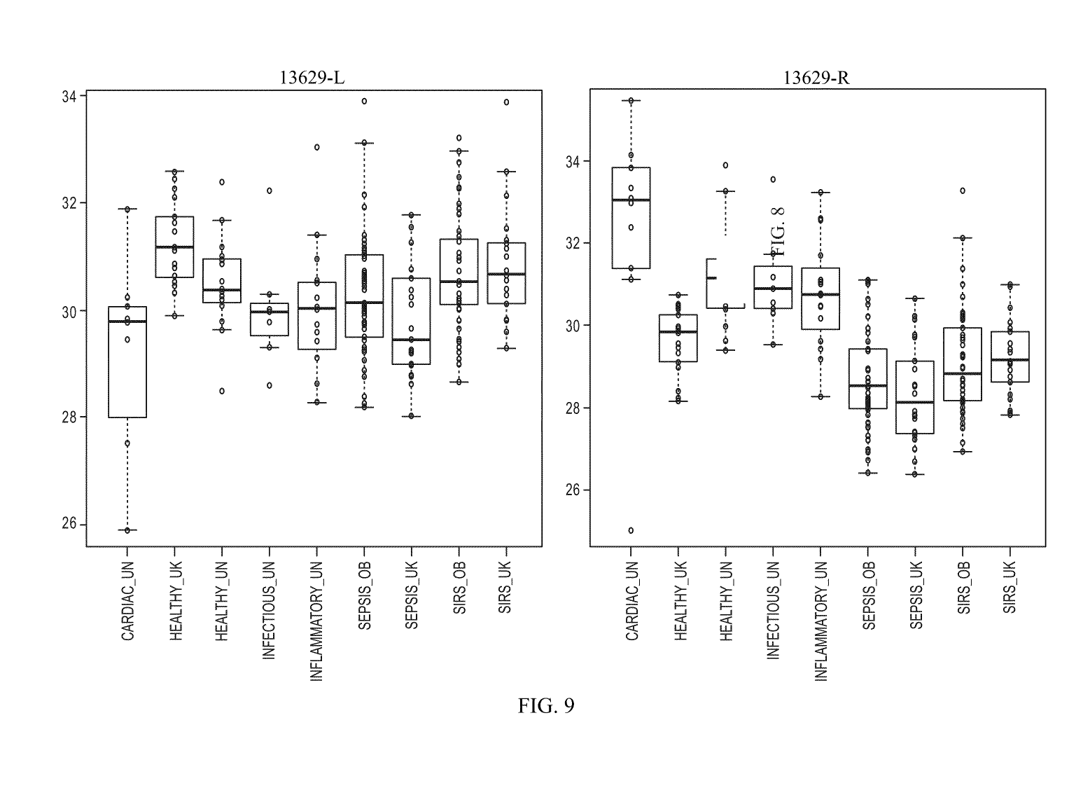

The expression levels of 13629-L and 13629-R were then measured in each patient group to determine if the cardiac and sepsis patients could be distinguished by included 13629-R in a panel. As shown in FIG. 9, it was found that 13629-L levels are higher in cardiac patients than healthy patients, similar to sepsis patients, but that 13629-R levels are much lower in cardiac patients than healthy patients, in contrast to the elevated levels of 13629-R in sepsis patients.

The results in FIG. 9 were confirmed by small RNA sequencing in samples from 11 of the cardiac patients. Table 8 shows the gender, age, and cardiac disease for each of the 11 patients, as well as the fold-difference in the number of reads of 13629-L and 13629-R versus healthy patients for each.

TABLE 8
13629-L and 13629-R sequencing in cardiac patients

| | | | | Fold-difference | |
|---|---|---|---|---|---|
| | | | | (cardiac versus healthy) | |
| Patient | Gender | Age | Cardiac condition | 13629-L | 13629-R |
| Cardiac 1 | M | 70 | Infarction | 1.47 | −5.99 |
| Cardiac 2 | M | 73 | Unstable angina | 2.06 | −2.81 |
| Cardiac 3 | M | 69 | Ischaemic heart | 1.97 | −13.93 |
| | | | disease | | |
| Cardiac 4 | F | 67 | Ischaemic heart | 4.52 | −2.89 |
| | | | disease | | |
| Cardiac 5 | M | 70 | Ischaemic heart | 4.73 | −3.15 |
| | | | disease | | |
| Cardiac 6 | M | 68 | Infarction | 3.41 | −6.19 |
| Cardiac 7 | F | 68 | Unstable angina | 2.30 | −2.05 |
| Cardiac 8 | M | 68 | Infarction | 2.56 | −3.68 |
| Cardiac 9 | M | 65 | Unstable angina | 2.57 | −10.77 |
| Cardiac 10 | M | 17 | Infarction | 2.16 | −2.35 |
| Cardiac 11 | F | 67 | Acute coronary | 2.32 | −2.14 |
| | | | syndrome | | |

It was found that levels of 13629-L were increased on average by about 2.7-fold in cardiac patients relative to healthy patients, while levels of 13629-R were reduced on average by about 5-fold in cardiac patients relative to healthy patients.

In view of the difference in 13629-L and 13629-R expression in cardiac patients versus sepsis patients, a panel comprising 13629-L, 13629-R, and miR-150 was tested on the samples from the various patient groups, including cardiac patients, healthy patients, patients with infectious diseases, patients with inflammatory conditions, sepsis patients, and SIRS patients. In this experiment, a linear discriminant model was used for the analysis. The coefficients of linear discriminants were determined to be −0.07278265 for 13629-L, −0.57562078 for 13629-R, and 0.67972131 for miR-150 for this experiment. Thus, the LDA score=−0.07278265*Ct_{—}13629-L−0.57562078*Ct_{—}13629-R+0.67972131*Ct_miR-150.

The results of that experiment are shown in FIG. 10. Including 13629-R along with 13629-L and miR-150 was effective to distinguish the cardiac patients from the sepsis patients in that experiment.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that changes can be made without departing from the spirit and scope of the invention(s).

1.-57. (canceled)

58. A method for detecting the presence of sepsis in a subject, comprising:
(a) hybridizing a first target RNA in a sample from the subject with a first probe and hybridizing a second target RNA in the sample with a second probe,
wherein the first probe comprises a sequence of at least 11 contiguous nucleotides that is identical or complementary to a sequence of at least 11 contiguous nucleotides of SEQ ID NO: 37,
wherein the second probe comprises a sequence of at least 11 contiguous nucleotides that is identical or complementary to a sequence of at least 11 contiguous nucleotides of SEQ ID NO: 44,
wherein each of the first and second probes consists of fewer than 150 nucleotides, and
wherein each of the first and second probes comprises at least one detectable moiety selected from fluorophore, electron spin label, biotin, horseradish peroxidase, radiolabel, and affinity-enhancing nucleotide analog; and
(b) detecting a level of each of the first and second target RNAs,
wherein a level of the first target RNA in the sample that is greater than a normal level of the first target RNA and/or a level of the second target RNA in the sample that is less than a normal level of the second target RNA indicates the presence of sepsis in the subject.

59. The method of claim 58, wherein the first target RNA comprises a sequence that is complementary to the first probe and/or the second target RNA comprises a sequence that is complementary to the second probe.

60. The method of claim 58, wherein the first target RNA comprises at least 11 contiguous nucleotides of SEQ ID NO: 1 and/or the second target RNA comprises at least 11 contiguous nucleotides of SEQ ID NO: 25.

61. The method of claim 58, wherein the first target RNA is 13629 and/or the second target RNA is 14689.

62. The method of claim 58, wherein the target RNA comprises a nucleic acid selected from a target RNA, a DNA amplicon of the target RNA, and a complement of the target RNA.

63. The method of claim 58, wherein the method further comprises isolating the target RNA from the sample.

64. The method of claim 63, wherein the target RNA comprises RNA that has been separated from DNA.

65. The method of claim 58, wherein the first and/or the second target RNA is fewer than 30 nucleotides.

66. The method of claim 58, wherein the first and/or the second target RNA is a microRNA.

67. The method of claim 58, wherein the first and/or the second probe comprises a fluorescent dye and a quencher molecule.

68. The method of claim 58, wherein the first probe comprises a sequence that is not identical or complementary to SEQ ID NO: 37 and/or the second probe comprises a sequence that is not identical or complementary to SEQ ID NO: 44.

69. The method of claim 58, wherein the sample is a bodily fluid.

70. The method of claim 69, wherein the bodily fluid is selected from blood, urine, sputum, saliva, and mucus.

71. The method of claim 58, wherein the sample is a blood sample.

72. The method of claim 71, wherein the blood sample is selected from whole blood, blood cells, plasma, serum, and peripheral blood mononuclear cells.

73. The method of claim 58, wherein the normal level of the first and second target RNA is determined from a sample from a single healthy individual or as an average or range that is characteristic of normal levels found in samples from healthy individuals.

74. The method of claim 58, wherein the hybridizing step further comprises hybridizing a third target RNA in the sample from the subject with a third probe, wherein the third probe comprises a sequence of at least 8 contiguous nucleotides that is identical or complementary to a sequence of at least 8 contiguous nucleotides of SEQ ID NO: 41, wherein the third probe consists of fewer than 150 nucleotides, and wherein the third probe comprises at least one detectable moiety selected from fluorophore, electron spin label, biotin, horseradish peroxidase, radiolabel, and affinity-enhancing nucleotide analog; and
wherein the detecting step further comprises detecting a level of the third target RNA, wherein a level of the third target RNA in the sample that is less than a normal level of the third target RNA indicates the presence of sepsis in the subject.

75. The method of claim 74, wherein levels of at least five target RNAs are detected.

76. A method for assessing the effectiveness of a treatment for sepsis in a patient, comprising:
(a) hybridizing a first target RNA in a sample taken from the patient during the treatment with a first probe and hybridizing a second target RNA in the sample with a second probe,
wherein the first probe comprises a sequence of at least 11 contiguous nucleotides that is identical or complementary to a sequence of at least 11 contiguous nucleotides of SEQ ID NO: 37,
wherein the second probe comprises a sequence of at least 11 contiguous nucleotides that is identical or complementary to a sequence of at least 11 contiguous nucleotides of SEQ ID NO: 44,
wherein each of the first and second probes consists of fewer than 150 nucleotides, and
wherein each of the first and second probes comprises at least one detectable moiety selected from fluorophore, electron spin label, biotin, horseradish peroxidase, radiolabel, and affinity-enhancing nucleotide analog; and
(b) detecting a level of each of the first and second target RNAs, wherein a level of the first target RNA in the sample that is less than a level of the first target RNA before treatment and/or wherein a level of the second target RNA in the sample that is greater than a level of the second target RNA before treatment indicates the effectiveness of the treatment.

77. The method of claim 76, wherein the hybridizing step further comprises hybridizing a third target RNA in the sample with a third probe, wherein the third probe comprises a sequence of at least 8 contiguous nucleotides that is identical or complementary to a sequence of at least 8 contiguous nucleotides of SEQ ID NO: 41, wherein the third probe consists of fewer than 150 nucleotides, and wherein the third probe comprises at least one detectable moiety selected from fluorophore, electron spin label, biotin, horseradish peroxidase, radiolabel, and affinity-enhancing nucleotide analog; and
wherein the detecting step further comprises detecting a level of the third target RNA, wherein a level of the third target RNA that is greater than a level of the third target RNA before treatment indicates the effectiveness of the treatment.


SEQUENCE LISTING (59 sequences)

SEQ ID NO: 1 - DNA, 22 bp, Homo sapiens
tctgatcaggcaaaattgcaga

SEQ ID NO: 2 - DNA, 88 bp, Homo sapiens
gctctgtgattgcctctgatcaggcaaaattgcagactgtcttcccaaatagcctgcaactttgcctgatcagaggcagtcacagagc

SEQ ID NO: 3 - DNA, 78 bp, Homo sapiens
gtgattgcctctgatcaggcaaaattgcagactgtcttcccaaatagcctgcaactttgcctgatcagaggcagtcac

SEQ ID NO: 4 - DNA, 22 bp, Homo sapiens
cctgcaactttgcctgatcaga

SEQ ID NO: 5 - DNA, 22 bp, Homo sapiens
tgatcaggcaaaattgcagact

SEQ ID NO: 6 - DNA, 22 bp, Homo sapiens
agctctagaaagattgttgacc

SEQ ID NO: 7 - DNA, 91 bp, Homo sapiens
ggttagcacagagtgggagctctagaaagattgttgaccaatcatcttattgactagaccatctttctagagtataactattttggacacc

SEQ ID NO: 8 - DNA, 21 bp, Homo sapiens
tagaccatctttctagagtat

SEQ ID NO: 9 - DNA, 23 bp, Homo sapiens
agctctagaaagattgttgacca

SEQ ID NO: 10 - DNA, 21 bp, Homo sapiens
agctctagaaagattgttgac

SEQ ID NO: 11 - DNA, 20 bp, Homo sapiens
agctctagaaagattgttga

SEQ ID NO: 12 - DNA, 22 bp, Homo sapiens
tctcccaacccttgtaccagtg

SEQ ID NO: 13 - DNA, 84 bp, Homo sapiens
ctccccatggccctgtctcccaacccttgtaccagtgctgggctcagaccctggtacaggcctgggggacagggacctggggac

SEQ ID NO: 14 - DNA, 22 bp, Homo sapiens
ctggtacaggcctgggggacag

SEQ ID NO: 15 - DNA, 23 bp, Homo sapiens
tctcccaacccttgtaccagtgc

SEQ ID NO: 16 - DNA, 21 bp, Homo sapiens
tctcccaacccttgtaccagt

SEQ ID NO: 17 - DNA, 20 bp, Homo sapiens
tctcccaacccttgtaccag

SEQ ID NO: 18 - DNA, 19 bp, Homo sapiens
tctcccaacccttgtacca

SEQ ID NO: 19 - DNA, 23 bp, Homo sapiens
tctcccaacccttgtaccagtga

SEQ ID NO: 20 - RNA, 22 nt, Homo sapiens
caacggaaucccaaaagcagcu

SEQ ID NO: 21 - RNA, 92 nt, Homo sapiens
cggcuggacagcgggcaacggaaucccaaaagcagcuguugucuccagagcauuccagcugcgcuuggauuucguccccugcucuccugccu

SEQ ID NO: 22 - RNA, 22 nt, Homo sapiens
gcugcgcuuggauuucgucccc

SEQ ID NO: 23 - RNA, 23 nt, Homo sapiens
caacggaaucccaaaagcagcug

SEQ ID NO: 24 - RNA, 24 nt, Homo sapiens
caacggaaucccaaaagcagcugu

SEQ ID NO: 25 - RNA, 22 nt, Homo sapiens
ugcccugccuguuuucuccuuu

SEQ ID NO: 26 - RNA, 68 nt, Homo sapiens
ucccugcccugccuguuuucuccuuugugauuuuaugagaacaaaggaggaaauaggcaggccaggga

SEQ ID NO: 27 - RNA, 22 nt, Homo sapiens
aaaggaggaaauaggcaggcca

SEQ ID NO: 28 - DNA, 24 bp, Homo sapiens
tgccctgcctgttttctcctttgt

SEQ ID NO: 29 - DNA, 23 bp, Homo sapiens
tgccctgcctgttttctcctttg

SEQ ID NO: 30 - DNA, 21 bp, Homo sapiens
tgccctgcctgttttctcctt

SEQ ID NO: 31 - RNA, 23 nt, Homo sapiens
ucucacacagaaaucgcacccgu

SEQ ID NO: 32 - RNA, 99 nt, Homo sapiens
gaaacugggcucaaggugaggggugcuaucugugauugagggacaugguuaauggaauugucucacacagaaaucgcacccgucaccuuggccuacuua

SEQ ID NO: 33 - RNA, 21 nt, Homo sapiens
aggggugcuaucugugauuga

SEQ ID NO: 34 - DNA, 24 bp, Homo sapiens
tctcacacagaaatcgcacccgtc

SEQ ID NO: 35 - DNA, 22 bp, Homo sapiens
tctcacacagaaatcgcacccg

SEQ ID NO: 36 - DNA, 2668 bp, Homo sapiens
ctctctggataggaagaaatatagtagaaccctttgaaaatggatattttcacatattttcgttcagatacaaaagctggcagttactgaaataaggacttgaagttccttcctcttttttttatgtcttaagagcaggaaataaagagacagctgaaggtgtagccttgaccaactgaaagggaaatcttcatcctctgaaaaaacatatgtgattctcaaaaaacgcatctggaaaattgataaagaagcgattctgtagattctcccagcgctgttgggctctcaattccttctgtgaaggacaacatatggtgatggggaaatcagaagctttgagaccctctacacctggatatgaatcccccttctaatacttaccagaaatgaaggggatactcagggcagagttctgaatctcaaaacactctactctggcaaaggaatgaagttattggagtgatgacaggaacacgggagaacaatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttcttgccccagtctcagctgccaaagtgatgcacaaagtccagcggtaacctggtacaagaatggaaaactcctctctgtggaaaggagcaaccgaatcgtagtggatgaagtttatgactatcaccagggcacatatgtatgtgattacactcagtcggatactgtgagttcgtggacagtcagagctgttgttcaagtgagaaccattgtgggagacactaaactcaaaccagatattctggatcctgtcgaggacacactggaagtagaacttggaaagcctttaactattagctgcaaagcacgatttggctttgaaagggtctttaaccctgtcataaaatggtacatcaaagattctgacctagagtgggaagtctcagtacctgaggcgaaaagtattaaatccactttaaaggatgaaatcattgagcgtaatatcatcttggaaaaagtcactcagcgtgatcttcgcaggaagtttgtttgctttgtccagaactccattggaaacacaacccagtccgtccaactgaaagaaaagagaggagtggtgctcctgtacatcctgcttggcaccatcgggaccctggtggccgtgctggcggcgagtgccctcctctacaggcactggattgaaatagtgctgctgtaccggacctaccagagcaaggatcagacgcttggggataaaaaggattttgatgctttcgtatcctatgcaaaatggagctcttttccaagtgaggccacttcatctctgagtgaagaacacttggccctgagcctatttcctgatgttttagaaaacaaatatggatatagcctgtgtttgcttgaaagagatgtggctccaggaggagtgtatgcagaagacattgtgagcattattaagagaagcagaagaggaatatttatcttgagccccaactatgtcaatggacccagtatctttgaactacaagcagcagtgaatcttgccttggatgatcaaacactgaaactcattttaattaagttctgttacttccaagagccagagtctctacctcatctcgtgaaaaaagctctcagggttttgcccacagttacttggagaggcttaaaatcagttcctcccaattctaggttctgggccaaaatgcgctaccacatgcctgtgaaaaactctcagggattcacgtggaaccagctcagaattacctctaggatttttcagtggaaaggactcagtagaacagaaaccactgggaggagctcccagcctaaggaatggtgaaatgagccctggagccccctccagtccagtccctgggatagagatgttgctggacagaactcacagctctgtgtgtgtgtgttcaggctgataggaaattcaaagagtctcctgccagcaccaagcaagcttgatggacaatggagtgggattgagactgtggtttagagcctttgatttcctggactggactgacggcgagtgaattctctagaccttgggtactttcagtacacaacacccctaagatttcccagtggtccgagcagaatcagaaaatacagctacttctgccttatggctagggaactgtcatgtctaccatgtattgtacatatgactttatgtatacttgcaatcaaataaatattattttattagaaa

SEQ ID NO: 37 - DNA, 22 bp, Artificial sequence
  Synthetic
tctgcaattttgcctgatcaga

SEQ ID NO: 38 - DNA, 22 bp, Artificial sequence
  Synthetic
agtctgcaattttgcctgatca

SEQ ID NO: 39 - DNA, 22 bp, Artificial sequence
  Synthetic
ggtcaacaatctttctagagct

SEQ ID NO: 40 - DNA, 21 bp, Artificial sequence
  Synthetic
gtcaacaatctttctagagct

SEQ ID NO: 41 - DNA, 22 bp, Artificial sequence
  Synthetic
cactggtacaagggttgggaga

SEQ ID NO: 42 - DNA, 22 bp, Artificial sequence
  Synthetic
agctgcttttgggattccgttg

SEQ ID NO: 43 - DNA, 23 bp, Artificial sequence
  Synthetic
cagctgcttttgggattccgttg

SEQ ID NO: 44 - DNA, 22 bp, Artificial sequence
  Synthetic
aaaggagaaaacaggcagggca

SEQ ID NO: 45 - DNA, 23 bp, Artificial sequence
  Synthetic
acgggtgcgatttctgtgtgaga

SEQ ID NO: 46 - DNA, 88 bp, Artificial sequence
  Synthetic
gctctgtgactgcctctgatcaggcaaagttgcaggctatttgggaagacagtctgcaattttgcctgatcagaggcaatcacagagc

SEQ ID NO: 47 - DNA, 78 bp, Artificial sequence
  Synthetic
gtgactgcctctgatcaggcaaagttgcaggctatttgggaagacagtctgcaattttgcctgatcagaggcaatcac

SEQ ID NO: 48 - DNA, 91 bp, Artificial sequence
  Synthetic
ggtgtccaaaatagttatactctagaaagatggtctagtcaataagatgattggtcaacaatctttctagagctcccactctgtgctaacc

SEQ ID NO: 49 - DNA, 84 bp, Artificial sequence
  Synthetic
gtccccaggtccctgtcccccaggcctgtaccagggtctgagcccagcactggtacaagggttgggagacagggccatggggag

SEQ ID NO: 50 - DNA, 92 bp, Artificial sequence
  Synthetic
aggcaggagagcaggggacgaaatccaagcgcagctggaatgctctggagacaacagctgcttttgggattccgttgcccgctgtccagccg

SEQ ID NO: 51 - DNA, 68 bp, Artificial sequence
  Synthetic
tccctggcctgcctatttcctcctttgttctcataaaatcacaaaggagaaaacaggcagggcaggga

SEQ ID NO: 52 - DNA, 99 bp, Artificial sequence
  Synthetic
taagtaggccaaggtgacgggtgcgatttctgtgtgagacaattccattaaccatgtccctcaatcacagatagcacccctcaccttgagcccagtttc

SEQ ID NO: 53 - DNA, 18 bp, Homo sapiens
accccactcctggtacca

SEQ ID NO: 54 - DNA, 17 bp, Homo sapiens
accccactcctggtacc

SEQ ID NO: 55 - DNA, 18 bp, Artificial sequence
  Synthetic
tggtaccaggagtggggt

SEQ ID NO: 56 - DNA, 93 bp, Homo sapiens
tacttatggcaccccactcctggtaccatagtcataagttaggagatgttagagctgtgagtaccatgacttaagtgtggtggcttaaacatg

SEQ ID NO: 57 - DNA, 86 bp, Homo sapiens
accaggatggccgagtggttaaggcgttggacttaagatccaatggacatatgtccgcgtgggttcgaaccccactcctggtacca

SEQ ID NO: 58 - DNA, 93 bp, Artificial sequence
  Synthetic
catgtttaagccaccacacttaagtcatggtactcacagctctaacatctcctaacttatgactatggtaccaggagtggggtgccataagta

SEQ ID NO: 59 - DNA, 86 bp, Artificial sequence
  Synthetic
tggtaccaggagtggggttcgaacccacgcggacatatgtccattggatcttaagtccaacgccttaaccactcggccatcctggt